SEQUENCE LISTING (2 sequences)

SEQ ID NO: 1 - protein, 215 aa, Unknown [sequence omitted]

SEQ ID NO: 2 - DNA, 13 bp, Artificial Sequence
  Description of Artificial Sequence Synthetic oligonucleotide
  modified_base (7)..(10): a, c, t, g, unknown or other
watcaannnnttr

(19) United States
(12) Patent Application Publication
(10) Pub. No.: US 2021/0139610 A1
(43) Pub. Date: May 13, 2021

(54) COMPOSITIONS AND METHODS FOR MEDIATING EPS

(71) Applicant: RESEARCH INSTITUTE AT NATIONWIDE CHILDREN'S HOSPITAL, Columbia, OH (US)

(72) Inventors: Steven David Goodman, Columbus, OH (US); Lauren Opremcak Bakaletz, Columbus, OH (US)

(73) Assignee: RESEARCH INSTITUTE AT NATIONWIDE CHILDREN'S HOSPITAL, Columbus, OH (US)

(21) Appl. No.: 17/256,640

(22) PCT Filed: Jun. 28, 2019
(86) PCT No.: PCT/US19/40008

§ 371 Date: Dec. 28, 2020

Related U.S. Application Data
(60) Provisional application No. 62/692,581, filed on Jun. 29, 2018.

(51) Int. Cl. C07K 16/44; A61K 45/06

(52) U.S. Cl. CPC C07K 16/44; A61K 45/06

(57) ABSTRACT

The disclosure relates to methods for inhibiting the stability of a biofilm comprising contacting the biofilm with an effective amount of an agent that interferes with the binding of a polyamine to DNA in the biofilm. Also provided herein are methods for treating a biofilm in a subject comprising administering to the subject infected with a biofilm an effective amount of an agent that interferes with the binding of a polyamine to the DNA in the biofilm. Further described herein are methods for treating a biofilm in a patient suffering from systemic lupus erythematosus (SLE) and/or cystic fibrosis (CF) comprising administering an effective amount of an agent that interferes with the conversion of B-DNA to Z-DNA in the biofilm or its local environment.

### CROSS-REFERENCE TO RELATED APPLICATIONS

This application claims priority under 35 U.S.C. § 119(e) to U.S. Provisional Application No. 62/692,581, filed Jun. 29, 2018, the content of which is hereby incorporated by reference in its entirety.

### FIELD

The present disclosure generally relates to the methods and compositions to remove or inhibit bacterial extracellular polymeric substance (EPS).

### BACKGROUND

Biofilms play a major role in medical, agricultural, and industrial settings. Biofilms are responsible for a significant portion of disease, both animal and plant, as well as for fouling of industrial equipment, and as such are the focus of intense research effort. Eradication or treatment of biofilms is particularly difficult to accomplish due to multiple factors, including production of an extracellular matrix that forms a physical barrier to antimicrobial effectors, altered physiology that is less susceptible to environmental stressors, and cooperative interactions among the constituents of the biofilm. The biofilm matrix is variably comprised of polysaccharides, proteins, and, perhaps universally, extracellular DNA (eDNA). The eDNA of a microbial biofilm is a critical constituent of the extracellular matrix that provides protection. Undermining the biofilm eDNA structure, via DNA degradation or removal of DNA binding proteins that stabilize the structure, results in catastrophic collapse of the biofilm and release of the resident bacterial into a more vulnerable state.

Bacteria are found in nature in two distinct states; planktonic bacteria are free living, while bacteria that develop into a community architecture are called biofilms (either on a surface or as aggregates). The CDC and NIH estimate that approximately 80% of all bacterial infections involve a necessary biofilm state. Dongari-Bagtzoglou et al. (2008) Expert Rev Anti Infect Ther. 6(2):201-8. These include otitis media (OM), chronic rhinosinusitis (CRS), chronic pulmonary infections, chronic wound infections, periodontitis, cystitis, and infections of medical implants and indwelling catheters, among many others. Indeed, one of the most common reasons to seek pediatric medical care is OM [caused by Nontypeable Haemphilus influenzae (NTHI) Streptococcus pneumoniae, Moraxella catarrhalis] and for adults, cystitis [e.g. Uropathogenic E. coli (UPEC)]; antibiotic prescriptions are accordingly most common for these complaints. Within the United States, it is estimated that 500,000 deaths annually are attributed to the direct consequences of bacterial biofilm infections. The economic impacts are staggering [$25B (billion) for chronic wounds, $14B for periodontitis, $5B for OM, and $1B for cystitis]. The worldwide prevalence of biofilm-mediated diseases, the increasing rate of antibiotic resistant bacterial infections, particularly among the high priority ESKAPE pathogens (Enterobacter spp., Staphylococcus aureus, Klebsiella pneumoniae, Acinetobacter baumannii, Pseudomonas aeruginosa, Enterococcus faecium), and the great financial burden create a critical need to develop novel approaches to treat recalcitrant infections caused by organized bacterial communities.

Thus, a need exists to break through the protective barrier of biofilms to treat or kill the associated bacterial infections and clear them from surfaces and in water systems.

### SUMMARY

The self-produced extracellular matrix (or extracellular polymeric substance, EPS) that protects bacteria resident within biofilms from immune clearance and antimicrobials is essential for pathogenic biofilms to cause chronic and recurrent infections, as biofilms serve as a recalcitrant reservoir of these disease-causing bacteria. The EPS constituents are specific to individual bacterial species, but universally contain extracellular DNA (eDNA) derived from the bacteria resident within the biofilm. Indeed, bacteria of varying genera typically enter into a shared community architecture of a multispecies biofilm, which requires the EPS to be both conducive structurally for all constituent species, but also to contain EPS components derived by, or usable to, all of the resident bacteria. In this regard, the EPS of single and multiple species biofilms contains scaffolded eDNA that appear to be the common structure of the underlying universal EPS. As disclosed herein, Applicants discovered that this eDNA-dependent structure is stabilized by the ubiquitous DNABII family of bacterial DNA-binding proteins. While Applicants have shown that exogenous DNA and DNABII proteins can drive free living (planktonic) bacteria into the community architecture of a biofilm, these two components are insufficient to recapitulate the signature eDNA scaffold.

Applicants disclose herein that polyamines are the third crucial component of the universal eDNA-DNABII dependent EPS. Polyamines are short positively charged organic molecules ubiquitous both intracellularly and extracellularly that, when bound to DNA, neutralize the polyanionic charge of nucleotide phosphates and allow DNA molecules to condense/aggregate. Importantly, Applicants disclose herein that polyamines can drive DNA from the most common right handed B-form into left handed Z-form DNA, which is nuclease resistant. Indeed, while nucleases can prevent bacterial biofilm formation, they cannot disrupt mature biofilms. As biofilms age, their acquisition of nuclease resistance is concomitant with both (1) an increase in polyamines and (2) the appearance of Z-form DNA.

Described herein are methods for inhibiting the stability of a biofilm, comprising, or alternatively consisting essentially of, or yet further consisting of contacting the biofilm with an effective amount of an agent that interferes with the binding of a polyamine to DNA in the biofilm, wherein the agent is not an HMGB1 protein, fragment or an equivalent of each thereof. In one aspect, the methods for inhibiting the stability of a biofilm, comprise, or alternatively consist essentially of, or yet further consist of a contacting the biofilm with an effective amount of one or more agents that interfere with the binding of a polyamine to the DNA in the biofilm. This disclosure also relates to methods for inhibiting the stability of a biofilm, comprising, or alternatively consisting essentially of, or yet further consisting of contacting the biofilm in vitro with an agent that interferes with the binding of a polyamine to the DNA in the biofilm, wherein the contacting comprises, or alternatively consists essentially of, or yet further consists of coating a surface with an effective amount of agent that depletes cations, wherein the agent is not an HMGB1 protein, fragment or an equivalent of each thereof. In one aspect, the methods for inhibiting the stability of a biofilm, may comprise, or alternatively consist essentially of, or yet further consist of contacting the biofilm in vitro with an effective amount of an agent that interferes with the binding of a polyamine to the DNA in the biofilm, wherein the contacting comprises, or alternatively consists essentially of, or yet further consists of coating a surface with an effective amount of one or more agents that depletes cations. The contacting may be in vitro or in vivo.

In one embodiment, the agent that interferes with the conversion of B-DNA to Z-DNA in the biofilm or its local environment. In a second embodiment, the agent comprises, or alternatively consists essentially of, or yet further consists of an anti-B-DNA antibody or fragment or derivative thereof. In a third embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In a fourth embodiment, the agent comprises, or alternatively consists essentially of, or yet further consists of chloroquine or a derivative thereof.

Further described herein are methods for inhibiting the stability of a biofilm, comprising, or alternatively consisting essentially of, or yet further consisting of contacting the biofilm in vitro with an effective amount of HMGB1 protein or biologically active fragment thereof and anti-B-DNA antibody or fragment or derivative thereof, wherein the contacting comprises, or alternatively consists essentially of, or yet further consists of coating a surface with an effective amount of HMGB1 protein or biologically active fragment thereof and anti-B-DNA antibody or fragment or derivative thereof. This disclosure also relates to methods for inhibiting the stability of a biofilm, comprising, or alternatively consisting essentially of, or yet further consisting of contacting the biofilm in vitro with an effective amount of chloroquine and anti-B-DNA antibody or fragment or derivative thereof, wherein the contacting comprises, or alternatively consists essentially of, or yet further consists of coating a surface with an effective amount of chloroquine and anti-B-DNA antibody or fragment or derivative thereof. The contacting may be in vitro or in vivo.

Also provided herein are methods for treating a biofilm in a subject, comprising, or alternatively consisting essentially of, or yet further consisting of administering to the subject infected with a biofilm an effective amount of an agent that interferes with the binding of a polyamine to the DNA in the biofilm, wherein the agent is not an HMGB1 protein, fragment or an equivalent of each thereof. In one aspect, the methods for treating a biofilm in a subject, comprise, or alternatively consist essentially of, or yet further consist of administering to the subject infected with a biofilm an effective amount of one or more agents that interfere with the binding of a polyamine to the DNA in the biofilm.

This disclosure also relates to methods for preventing the formation of a biofilm in a subject susceptible to developing a biofilm, comprising, or alternatively consisting essentially of, or yet further consisting of administering to the subject an effective amount of an agent that interferes with the binding of a polyamine to the DNA in the biofilm, wherein the agent is not an HMGB1 protein, fragment or an equivalent of each thereof. In one aspect, the methods for preventing the formation of a biofilm in a subject susceptible to developing a biofilm, comprising, or alternatively consisting essentially of, or yet further consisting of administering to the subject an effective amount of one or more agents that interfere with the binding of a polyamine to the DNA in the biofilm.

This disclosure further relates to methods for treating an infection caused by a bacterium that produces a biofilm in a subject in need thereof, the method comprising, or alternatively consisting essentially of, or yet further consisting of administering to the subject an effective amount of an agent that interferes with the binding of a polyamine to the DNA in the biofilm and an agent that inhibits the replication of the organism, wherein the agent is not an HMGB1 protein, fragment or an equivalent of each thereof. In one aspect, the methods for treating an infection caused by a bacterium that produces a biofilm in a subject in need thereof, the method comprising, or alternatively consisting essentially of, or yet further consisting of administering to the subject an effective amount of one or more agents that interfere with the binding of a polyamine to the DNA in the biofilm.

For any of the methods described above, the polyamine can be selected from the group of: putrescine, spermine, cadaverine, 1,3-diaminopropane or spermidine. In one embodiment, for the methods described above, the agent that interferes with the binding of a polyamine to DNA in the biofilm is a tRNA. In another embodiment, the agent is an inhibitor of polyamine synthesis or an agent that inhibits the binding of the polyamine to the DNA. In a second embodiment, the agent comprises, or alternatively consists essentially of, or yet further consisting of a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof. In a third embodiment, the agent comprises, or alternatively consists essentially of, or yet further consisting of an agent that depletes cations from the biofilm, optionally a cation exchange resin, an aminopolycarboxylic acid, a crown ether, an azacrown, or a cryptand. In a fourth embodiment, the agent that depletes cations from the biofilm are selected from the group of: sulfonate, sulfopropyl, phosphocellulose, P11 phosphocellulose, heparin sulfate, or a derivative or analog thereof. In a fifth embodiment, the agent that interferes with the conversion of B-DNA to Z-DNA in the biofilm or its local environment. In a sixth embodiment, the agent comprises, or alternatively consists essentially of, or yet further consists of an anti-B-DNA antibody or fragment or derivative thereof. In an eighth embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In a ninth embodiment, the agent comprises, or alternatively consists essentially of, or yet further consists of chloroquine or a derivative thereof. In one aspect, the agent that depletes cations from the biofilm has a net negative charge. In another aspect, the agent that depletes cations from the biofilm has a net neutral charge.

Also provided herein are methods for treating a biofilm in a patient suffering from systemic lupus erythematosus (SLE) and/or cystic fibrosis (CF), comprising, or alternatively consisting essentially of, or yet further consisting of administering an effective amount of an agent that interferes with the conversion of B-DNA to Z-DNA in the biofilm or its local environment, wherein the agent is not an HMGB1 protein, fragment or an equivalent of each thereof. Methods for treating a biofilm in a patient suffering from systemic lupus erythematosus (SLE) and/or cystic fibrosis (CF) and/or TB, comprising, or alternatively consisting essentially of, or yet further consisting of administering an effective amount of one or more agents that interfere with the conversion of B-DNA to Z-DNA in the biofilm or its local environment are disclosed herein, wherein the agent is not an HMGB1 protein, fragment or an equivalent of each thereof. In one aspect, the agents are administered in the absence of a DNAse. In one embodiment, the agent that interferes with the conversion of B-DNA to Z-DNA in the biofilm or its local environment. In a second embodiment, the agent comprises, or alternatively consists essentially of, or yet further consists of an anti-B-DNA antibody or fragment or derivative thereof. In a third embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In a fourth embodiment, the agent comprises, or alternatively consists essentially of, or yet further consists of chloroquine or a derivative thereof. In one aspect, the chloroquine derivative retains the capacity to intercalate between DNA bases.

Methods for treating a biofilm in a patient suffering from systemic lupus erythematosus (SLE) and/or cystic fibrosis (CF), comprising, or alternatively consisting essentially of, or yet further consisting of administering an effective amount of HMGB1 protein or biologically active fragment thereof and anti-B-DNA antibody or fragment or derivative thereof are also provided herein. In one aspect, the method for treating a biofilm in a patient suffering from systemic lupus erythematosus (SLE) and/or cystic fibrosis (CF) and/or tuberculosis (TB), the method which comprises, or alternatively consists essentially of, or yet further consists of administering an effective amount of chloroquine and anti-B-DNA antibody or fragment or derivative thereof. This disclosure also relates to methods for treating a biofilm producing infection incident to administration of a platinum-based chemotherapy in a patient receiving or having received the chemotherapy comprising, or alternatively consisting essentially of, or yet further consisting of administering an effective amount of an agent that interferes with the conversion of B-DNA to Z-DNA in the biofilm or its local environment, wherein the agent is not an HMGB1 protein, fragment or an equivalent of each thereof. In one aspect, the method comprises, or alternatively consists essentially of, or yet further consists of administering an effective amount of one or more agents that interfere with the conversion of B-DNA to Z-DNA in the biofilm or its local environment. In one aspect, the agents are administered in the absence of a DNAse. In a further aspect, the agent comprises, or alternatively consists essentially of, or yet further consists of chloroquine or a derivative thereof. In one particular aspect, the chloroquine derivative retains the capacity to intercalate between DNA bases. In yet a further aspect, the agent comprises, or alternatively consists essentially of, or yet further consists of an anti-B-DNA antibody or fragment or derivative thereof. In one embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof.

This disclosure further relates to methods for treating a biofilm producing infection incident to administration of a platinum-based chemotherapy in a patient receiving or having received the chemotherapy comprising, or alternatively consisting essentially of, or yet further consisting of administering an effective amount of HMGB1 protein or biologically active fragment thereof and anti-B-DNA antibody or fragment or derivative thereof. Methods for treating a biofilm producing infection incident to administration of a platinum-based chemotherapy in a patient receiving or having received the chemotherapy comprising, or alternatively consisting essentially of, or yet further consisting of administering an effective amount of chloroquine and anti-B-DNA antibody or fragment or derivative thereof are also provided herein.

The methods described above may further comprise, or alternatively consist essentially of, or yet further consist of contacting the biofilm, or alternatively administering to the subject, an effective amount of an agent that interferes with the binding of the eDNA to a DNA binding protein and/or an antibacterial agent, wherein the agent is not an HMGB1 protein, fragment or an equivalent of each thereof. In one aspect, the agent that interferes with the binding of the eDNA to the DNA binding protein comprises, or alternatively consists essentially of, or yet further consists of one or more of an anti-DNABII antibody, an anti-IHF antibody and/or an anti-HU antibody, or fragments of each thereof. In one embodiment, the agent that interferes with the binding of the eDNA to a DNA binding protein has a net negative charge. In a second embodiment, the agent that interferes with the binding of the eDNA to a DNA binding protein has a net neutral charge. In a third embodiment, the agent that interferes with the binding of the eDNA to a DNA binding protein has a net positive charge. In one aspect, the agents are administered in the absence of a DNAse. The methods described above may be performed in the absence of administration of a DNAse enzyme.

### BRIEF DESCRIPTION OF THE DRAWINGS

FIGS. 1A-1C: Polyamines modulate DNA structure. (FIG. 1A) Immunofluorescence microscopy image of mucosal biofilm EPS found in the chinchilla middle ear with acute OM due to NTHI (adapted from Goodman et al. (2011) Mucosal Immunol. 4(6):625-37). Probed for DNABII proteins (gray balls) and eDNA (white regions). DNABII localizes to eDNA strand vertices in biofilms in vivo. (FIG. 1B) Chemical structure of common polyamines (adapted from Di Martino et al. (2013) Int J Med Microbiol. 303(8):484-91). (FIG. 1C) Atomic force microscopy image of DNA alone or DNA after incubation with polyamines (adapted from Iacomino et al. (2011) Biomacromolecules. 12(4):1178-86). Polyamines induce thick fiber formation and structural complexity in eDNA.

FIGS. 2A-2B: Polyamines induce eDNA scaffold structure. (FIG. 2A) Immunofluorescence CLSM image of mucosal biofilm EPS found in the chinchilla middle ear with acute OM due to NTHI. Probed for polyamines (white dots) [putrescine (Put), cadaverine (Cad), spermidine (Spd)] and eDNA (white), counterstained with DAPI (gray regions). Polyamines localize to eDNA strands in biofilms that were formed in vivo; spermidine is the most prevalent polyamine in biofilm EPS. (FIG. 2B) Transmission electron microscopy images of complexes of DNA (5 μM) and spermidine (700 μM) (top) and DNA (5 μM), spermidine (700 μM), and HU (50 nM) (bottom) (adapted from Sarkar et al. (2007) Nucleic Acids Res. 35(3):951 61). Similar structures are formed by DNA-polyamine-IHF complexes. Sarkar et al. (2009) Biochemistry. 48(4):667-75. Polyamines induce DNA condensation and combine with DNABII proteins to form thick fibers.

FIGS. 3A-3C: Polyamine synthesis inhibitor reduces biofilm formation. (FIG. 3A) COMSTAT quantification of LIVE/DEAD® stained NTHI biofilms grown in the presence of dicyclohexylamine (DCHA, 50 μM), a spermidine (Spd) synthase inhibitor, Spd (1 mM), or both. Bars represent the SEM. Statistical significance compared to control was assessed by unpaired t-tests, *P<0.05. Average thickness of biofilms was decreased by DCHA, while simultaneous addition of exogenous Spd restored biofilm formation. (FIG. 3B) Immunofluorescence microscopy images of eDNA scaffold structure in NTHI in vitro biofilms grown for 3 h in the presence of DCHA (50 μM). Probed for dsDNA (white regions). eDNA scaffold structure production is greatly reduced by inhibition of polyamine synthesis. (FIG. 3C) Immunofluorescence CLSM images of NTHI in vitro biofilms grown for 40 h in the presence of DCHA. Probed for Spd (white dots in FIG. 3C lower panel) and counterstained with DAPI (gray regions). DCHA inhibits polyamine incorporation into the biofilm EPS.

FIGS. 4A-4B: Anti-DNABII disrupts DNABII-polyamine (PA) dependent structures. (FIG. 4A) DNA structures were formed by incubating spermidine (300 μM) and HU (1 μM) in buffer that contained genomic DNA (gDNA; 2 μg/ml) for 40 h. Immunofluorescence CLSM images of DNABII-polyamine dependent DNA structures. Probed for DNABII proteins (white; indicated on the right side of the image) and counterstained with DAPI (white; indicated on the left side of the image). DNABII polyamine dependent DNA structures incorporate DNABII proteins. (FIG. 4B) EPS structures were formed as in (FIG. 4A) for 24 h and treated for additional 16 h with 1:50 dilution of DNABII antiserum (indicated below the image). Fluorescence CLSM images stained with DAPI (white). DNABII-polyamine dependent DNA structures require DNABII proteins.

FIG. 5: The cation exchanger phosphocellulose (P11) disrupts NTHI biofilm formation. NTHI biofilm growth was initiated in the basolateral chamber of a transwell plate system while P11 (1%) was added to the apical chamber. Spermidine (1 mM) and HU (1 μM) were added at seeding, and maintained for 16 h. Biofilms were visualized via CLSM and analyzed by COMSTAT. Average thickness (not shown) showed identical trends. Bars represent the SEM. Statistical significance compared to control was assessed by unpaired t-tests, *P<0.05; **P<0.01. P11 prevents biofilm formation. Exogenous spermidine and HU together restores biofilm development, but not either alone (not shown), which suggests that P11 antibiolfilm activity is the result of titrating structural components (polyamines and DNABII proteins) from the biofilm EPS.

FIG. 6: Mature biofilms are resistant to disruption by DNase. DNase (Pulmozyme; 5 units) was added at seeding (prevention) or at 24 h (disruption) to the respective in vitro preformed NTHI and UPEC biofilms. After a total of 40 h, biofilms were stained with LIVE/DEAD®, visualized via CLSM and analyzed by COMSTAT. Bars represent the SEM. Statistical significance compared to control was assessed by unpaired t-tests, *P<0.05; **P<0.01. DNase can prevent, but not disrupt extant biofilms.

FIGS. 7A-7D: DNABII proteins and polyamines (PAs) interact synergistically to induce DNase resistance. (FIG. 7A) Immunofluorescence CLSM images of NTHI in vitro biofilms grown for 40 h. Probed for spermidine (dark gray balls), HU (light gray balls), and counterstained with DAPI (gray regions). Polyamines and DNABII proteins co-localize in biofilm EPS in vitro (white balls). (FIG. 7B) Immunofluorescence CLSM image of mucosal biofilm EPS found in the chinchilla middle ear with acute OM due to NTHI. Probed for putrescine (white balls) and HU (light gray balls), counterstained with DAPI (dark gray regions). Polyamines and DNABII proteins co-localize on eDNA strands in biofilms in vivo (white regions). (FIG. 7C) Increasing levels of spermidine (Spd) and HU, separately or together, were incubated with genomic DNA (2 μg/ml) for 1.5 h at 37° C., followed by treatment with Pulmozyme® for 20 min. DNA degradation was assayed using agarose gel electrophoresis. Spd and HU synergistically protect genomic DNA from DNase digestion. (FIG. 7D) Spd (300 μM) and HU (1 μM) were incubated with genomic DNA (2 μg/ml) for 40 h, followed by Pulmozyme® treatment for 20 min. Structures were stained with LIVE/DEAD® and imaged by CLSM (white). HU-Spd dependent DNA structures are resistant to DNase treatment.

FIGS. 8A-8B: DNABII and polyamines combine to convert B- to ZDNA form. (FIG. 8A) DNABII-polyamine dependent DNA structures were formed by incubating genomic DNA (2 μg/ml) with HU (1 μM) and Spermidine (300 μM) for 16 h. Immunofluorescence CLSM images of DNABII-polyamine dependent DNA structures. Probed for Z-DNA (white) and stained with DAPI (dark gray). Polyamines and DNABII proteins synergize to induce B- to Z-DNA conversion. (FIG. 8B) Top: Circular dichroism spectrum of a B-DNA substrate being converted to Z-DNA by increasing concentrations of Z-DNA catalyst (adapted from Jang et al. (2015) Sci Rep. 5:9943). Note inversion of negative peak around 250 nm and positive peak around 280 nm. Bottom: Poly (dGdC) DNA (20 μg/ml) was incubated with HU (15 μM) for 2 h and CD spectra collected. HU shifts CD spectrum of poly (dGdC) towards a Z-DNA signature.

FIG. 9: Z-DNA is present within the biofilm EPS of multiple human pathogens. Top: Immunofluorescence CLSM images of 40 h biofilms of the indicated bacteria, probed with no primary antibody (No 1°) or with Z-DNA antibody (white). Z-DNA is a component of the EPS of multiple bacterial biofilms at different steady state levels. Bottom: Immunofluorescence CLSM images of indicated biofilms probed with HU (white) and spermidine (dark gray) antibodies, co-localization is (white). DNABII and polyamine components co-localize in the EPS of multiple bacterial biofilms at steady state levels that correlate with Z-DNA abundance.

FIG. 10: Z-DNA and polyamines increase in abundance as UPEC and NTHI biofilms mature. Immunofluorescence CLSM images of UTI89 and NTHI in vitro biofilms at various stages of formation, probed with anti-Z-DNA (white), or anti-spermidine (dark gray). Mature biofilms incorporate an increasing amount of Z-DNA (white) and spermidine (dark gray) within the biofilm EPS over time.

FIG. 11: HU is required for B- to Z-DNA conversion and incorporation of polyamines into the biofilm EPS. Immunofluorescence CLSM images of 40 h NTHI wild type and the ΔHU mutant in vitro biofilms, probed with spermidine (dark gray) or anti-Z-DNA (white). In the absence of HU, polyamines and Z-DNA decrease in abundance within the biofilm EPS.

FIG. 12: Non-typeable Haemophilus influenzae biofilms were grown for 40 h in supplemented BHI media in 8 well chambered cover glass slides at 37° C. and 5% CO₂. Biofilms were washed, probed with indicated primary antibodies, a fluorescent secondary antibody (dark gray dots), and stained with DAPI (gray regions). Note: minimal dark gray staining in bottom leftmost image represents background. Putrescine, spermidine, and spermine were all present throughout the biofilm matrix.

FIG. 13: Non-typeable Haemophilus influenzae were grown for 16 h in supplemented BHI media containing additives as indicated above in 96 well plates at 37° C. and 5% CO₂. Growth was quantified by spectrophotometric absorbance at 490 nm (left) and by enumeration of colony-forming units (right). The spermidine synthase inhibitor did not affect normal growth.

FIG. 14: Non-typeable Haemophilus influenzae biofilms were grown for 40 h in supplemented BHI medium containing additives as indicated below each bar in 8 well chambered coverglass slides at 37° C. and 5% CO₂. Biofilms were washed, stained with LIVE/DEAD®, fixed, and imaged by CLSM. Biofilm parameters were quantified using COMSTAT software. Dicyclohexylamine inhibited biofilm biogenesis, while exogenous spermidine was able to rescue biofilm growth.

FIG. 15: Non-typeable Haemophilus influenzae biofilms were grown for 40 h in supplemented BHI media containing additives as indicated above in 8 well chambered coverglass slides at 37° C. and 5% CO₂. Biofilms were washed, probed with primary antibodies directed towards spermidine, a fluorescent secondary antibody (dark gray dots), and stained with DAPI (gray regions). The spermidine synthase inhibitor reduced spermidine presence in the biofilm matrix.

FIG. 16: Surface attached non-typeable Haemophilus influenzae were grown for 3 h in supplemented BHI media containing additives as indicated above in Fluorodish coverglass dishes at 37° C. and 5% CO₂. Biofilms were washed, probed with primary antibodies directed towards double stranded DNA and a fluorescent secondary antibody (white). The spermidine synthase inhibitor reduced both the presence and complexity of the extracellular DNA structure within the biofilm matrix.

FIGS. 17A-17D: Spermidine is present within the biofilm EPS formed by multiple human pathogens. (FIG. 17A). Immunofluorescence CLSM images of indicated biofilms probed with HU (light gray) and spermidine (dark gray) antibodies, co-localization is white. DNABII and polyamine components co-localize in the EPS of multiple bacterial biofilms at steady state levels. (FIG. 17B) Dicyclohexylamine (DCHA) inhibition of spermidine biosynthesis reduces spermidine levels in NTHI and UPEC biofilms indicated by a decrease in IF signal, and results in a significantly decreased average thickness compared to sBHI control (FIG. 17C). UPEC represented as percent change average thickness compared to LB control (FIG. 17D).

FIGS. 18A-18B: Phosphocellulose has a dose-dependent negative effect on biofilm formation and preformed NTHI biofilm stability in vitro. (FIG. 18A) Biofilm growth was initiated then maintained for 24 hrs then treated for 16 hrs with 0 (SBHI Control), 0.1%, 1%, and 5% (w/v) of Phoshocellulose (P11). (FIG. 18B) Biofilm growth was initiated then maintained for 40 hrs in the presence of 0 (sBHI Control), 0.1%, 1%, and 5% (w/v) of (P11). Biofilms were washed with saline and stained with LIVE/DEAD® stain. Images were analyzed by COMSTAT to calculate average thickness and biomass. All images were captured with a 63× objective.

FIG. 19: Heparin sepharose has a negative effect on NTHI biofilm formation in vitro. Biofilm growth was initiated then maintained for 40 hrs in the presence of 0 (sBHI Control) or 5% (w/v) of Heparin Sepharose resin. Biofilms were washed with saline and stained with LIVE/DEAD® stain. Images were analyzed by COMSTAT to calculate average thickness and biomass. All images were captured with a 63× objective.

FIG. 20: Exogenous addition of HU rescues the negative effect of phosphocellulose on NTHI biofilm stability in vitro. Biofilm growth was initiated and maintained for 24 hrs, then treated for 16 hrs as indicated. Biofilms were washed with saline and stained with LIVE/DEAD® stain. Images were analyzed by COMSTAT to calculate average thickness and biomass and compared to the sBHI control. All images were captured with a 63× objective. Bars represent the SEM. Statistical significance compared to control was assessed by unpaired t-tests, *P<0.05; **P<0.01.

FIG. 21: Exogenous addition of MgCl₂ rescues the negative effect of phosphocellulose on NTHI biofilm stability in vitro. Biofilm growth was initiated and maintained for 24 hrs then treated for 16 hrs as indicated. Biofilms were washed with saline and stained with LIVE/DEAD® stain. Images were analyzed by COMSTAT to calculate average thickness and biomass. All images were captured with a 63× objective.

FIG. 22: Exogenous addition of Spermidine rescues the negative effect of phosphocellulose on NTHI biofilm stability in vitro. Biofilm growth was initiated then maintained for 24 hrs then treated for 16 hrs as indicated. Biofilms were washed with saline and stained with LIVE/DEAD® stain. Images were analyzed by COMSTAT to calculate average thickness and biomass. All images were captured with a 63× objective.

FIG. 23: Cation depletion effects of P11 phosphocellulose does not require direct contact with biofilm. Biofilm growth was initiated in the basolateral chamber of a transwell plate system while 0, 0.5, 1, or 1.5% (w/v) P11 Phosphocellulose was added to the apical chamber and was maintained for 16 hrs. Biofilms were washed with saline and stained with LIVE/DEAD® stain. Images were analyzed by COMSTAT to calculate average thickness and biomass and were compared to the sBHI control. All images were captured with a 63× objective. Bars represent the SEM. Statistical significance compared to control was assessed by unpaired t-tests, *P<0.05; **P<0.01.

FIG. 24: Exogenous addition of spermidine reduces the cation depletion effects of P11 phosphocellulose without requiring direct contact with biofilm. Biofilm growth was initiated in the basal chamber of a transwell plate system while 0 or 1.5% (w/v) P11 Phosphocellulose was added to the apical chamber and was maintained for 16 hrs in the presence of 100, 500 or 1000 uM Spermidine Biofilms were washed with saline and stained with LIVE/DEAD® stain. Images were analyzed by COMSTAT to calculate average thickness and biomass and were compared to the sBHI control. All images were captured with a 63× objective. Bars represent the SEM. Statistical significance compared to control was assessed by unpaired t-tests, *P<0.05; **P<0.01. Brackets indicate statistical comparison between conditions.

FIG. 25: Exogenous addition of spermidine and DNABII reduces the cation depletion effects of P11 phosphocellulose without direct contact with biofilm. Biofilm growth was initiated in the basolateral chamber of a transwell plate system while 0 or 1.5% (w/v) P11 Phosphocellulose was added to the apical chamber and was maintained for 16 hrs in the presence of 100 uM spermidine or 500 nM HU or in combination. Biofilms were washed with saline and stained with LIVE/DEAD® stain. Images were analyzed by COMSTAT to calculate average thickness and biomass and were compared to the sBHI control. All images were captured with a 63× objective. Bars represent the SEM. Statistical significance compared to control was assessed by unpaired t-tests, *P<0.05; **P<0.01. Brackets indicate statistical comparison between conditions.

FIGS. 26A-26B: Coating abiotic surfaces with cation exchange resin prevents biofilm formation in a dose-dependent manner. Chamber slides were coated with P11 phosphocellulose (FIG. 26A) or heparin sepharose (FIG. 26B) solutions as indicated. Biofilm growth was initiated and maintained for 40 hrs on coated slides. Biofilms were washed with saline and stained with LIVE/DEAD® stain. Images were analyzed by COMSTAT to calculate average thickness and biomass. All images were captured with a 63× objective. Bars represent the SEM.

FIG. 27: Mature biofilms are resistant to disruption by DNase. DNase (Pulmozyme; 5 units) was added at seeding (prevention) or at 24 h (disruption) to the respective in vitro pre-formed NTHI or UPEC biofilms. After a total of 40 h, biofilms were stained with LIVE/DEAD®, visualized via CLSM and analyzed by COMSTAT. Bars represent the SEM. Similar trends were observed for biomass. Statistical significance compared to control was assessed by unpaired t-tests, *P<0.05; **P<0.01. DNase can prevent, but not disrupt extant biofilms.

FIG. 28: Z-DNA and polyamines increase in abundance as UPEC and NTHI biofilms mature. Immunofluorescence CLSM images of UPEC and NTHI biofilms formed in vitro at various stages of maturation, probed with anti-Z-DNA (light gray), or anti-spermidine (dark gray). Mature biofilms incorporate an increasing amount of Z-DNA (light gray) and spermidine (dark gray) within the biofilm EPS over time.

FIG. 29: Z-DNA is present in mature pathogenic fungal biofilms. Immunofluorescence CLSM images of biofilms formed by Candida albicans in vitro, counterstained with DAPI, and probed with anti-B-DNA, anti-Z-DNA, or no primary (light gray). Mature fungal biofilms incorporate Z-DNA (light gray) within the biofilm EPS.

FIG. 30: Anti-Z-DNA antibodies stimulate biofilm biogenesis. Anti-Z-DNA antibodies (1 mg) were added to NTHI in vitro biofilms at seeding. After 16 h, biofilms were stained with LIVE/DEAD®, visualized via CLSM and analyzed by COMSTAT. Bars represent the SEM. Similar trends were observed for biomass. Statistical significance compared to control was assessed by paired t-tests. Anti-Z-DNA antibodies stabilize the biofilm extracellular matrix, stimulating biofilm biogenesis whereas antibodies to B-DNA (e.g. anti-dsDNA) do not stimulate biofilm biogenesis.

FIG. 31: DNase degrades B-DNA, but not Z-DNA, within the biofilm extracellular matrix. DNase (Pulmozyme; 5 units) was added at 24 h after biofilm seeding to in vitro pre-formed NTHI biofilms. After a total of 40 h, biofilms were probed with anti-Z-DNA, anti-B-DNA, or no primary antibodies, which were revealed via use of corresponding secondary fluorescent antibodies and visualized via CLSM. DNase treatment degrades B-DNA form eDNA structures, but reveals extensive Z-DNA form eDNA within the biofilm extracellular matrix.

FIG. 32: Z-DNA formation protects DNA from nuclease degradation. A poly(dG-dC) substrate was incubated with salt activated nuclease (SAN) or DNase I in increasing concentrations of NaCl, spermine, or spermidine as indicated above. Degradation products were visualized by gel electrophoresis. High salt and polyamines protect DNA from degradation through conversion to the Z-DNA form.

FIG. 33: DNABII proteins and polyamines co-localize within the biofilm extracellular matrix. Non-typeable Haemophilus influenzae biofilms were grown in supplemented BHI media in 8-well chambered coverglass slides at 37° C. and 5% CO₂. Biofilms were washed, probed with primary antibodies against DNABII proteins (light gray) or polyamines (dark gray), a fluorescent secondary antibody, stained with DAPI (gray regions), and visualized via fluorescence microscopy. Polyamines co-localized with HU but not with IHF in the biofilm matrix. An NTHI strain unable to produce HU had reduced accumulation of polyamines in the biofilm matrix. DNABII proteins and polyamines interact to stabilize the biofilm extracellular matrix.

FIGS. 34A-34B: DNABII proteins protect DNA by shifting to Z-DNA form. (FIG. 34A) A poly(dG-dC) substrate was incubated with DNase I in increasing concentrations of NTHi HU as indicated above. Degradation products were visualized by gel electrophoresis. HU protected DNA from degradation. (FIG. 34B) Top: Circular dichroism spectrum of a B-DNA substrate being converted to Z-DNA by increasing concentrations of a Z-DNA catalyst (adapted from Rahmouni (1992) Mol Microbiol. 6(5):569-72). Note inversion of negative peak around 250 nm and positive peak around 280 nm. Bottom: Poly(dGdC) DNA (5 μg) was incubated with HU (15 μM) for 2 h and CD spectra collected. HU shifts the CD spectrum of poly(dGdC) towards a Z-DNA signature.

FIGS. 35A-35D: DNABII proteins and polyamines (PAs) interact synergistically to induce DNase resistance. (FIG. 35A) Increasing levels of spermidine (Spd) and HU, separately or together, were incubated with genomic DNA (gDNA; 2 μg/ml) for 1.5 h at 37° C., followed by treatment with Pulmozyme® for 20 min. DNA degradation was assayed using agarose gel electrophoresis. Spd and HU synergistically protect genomic DNA from DNase digestion. (FIG. 35B) Spd (300 μM) and HU (1 μM) were incubated with genomic DNA (2 μg/ml) for 40 h, followed by Pulmozyme® treatment for 20 min. Structures were stained with LIVE/DEAD® and imaged by CLSM (white). (FIG. 35C) Immunofluorescence CLSM image of mucosal biofilm EPS found in the chinchilla middle ear with experimental OM due to NTHI. Probed for putrescine (dark gray) and HU (light gray), counterstained with DAPI (gray). HU-Spd dependent DNA structures are resistant to DNase treatment. Polyamines and DNABII proteins co-localize on eDNA strands in biofilms in vivo (white). (FIG. 35D): Increasing concentrations of DNase were added for 16 h at seeding (Prevention) or at 24 h (Disruption) to NTHI or UPEC biofilms. Biofilms were stained, fixed, visualized via CLSM, and analyzed by COMSTAT. Bars represent the SEM. Statistical significance compared to Control (No DNase) was assessed by unpaired t-tests, **P<0.01. DNase can prevent formation of, but not disrupt extant biofilms.

FIG. 36: Z-DNA is present within the biofilm EPS of multiple human pathogens. Top: Immunofluorescence CLSM images of 40 h biofilms formed by the indicated bacteria, probed with either no primary antibody (No 1°) or with Z-DNA-specific antibody (light gray). Z-DNA is a component of the EPS of multiple bacterial biofilms at different steady state levels. Bottom: Immunofluorescence CLSM images of indicated biofilms probed with HU (light gray) and spermidine (dark gray) antibodies, co-localization is (white). DNABII and polyamine components co-localize in the EPS of multiple bacterial biofilms at steady state levels that correlate with Z-DNA abundance.

FIG. 37: HMGB1 destabilizes Z-DNA structures in the biofilm extracellular matrix. Non-typeable Haemophilus influenzae biofilms were grown in supplemented BHI media in 8 well chambered coverglass slides at 37° C. and 5% CO₂. After 24 h, biofilms were treated as indicated above. After 40 h, biofilms were washed, probed with primary antibodies directed against Z-DNA (light gray dots), a fluorescent secondary antibody, stained with DAPI (gray regions), and visualized via fluorescence microscopy. HMGB1 treatment reduced Z-DNA structures in the biofilm matrix.

FIGS. 38A-38C: HMGB1 displaces DNABII proteins thereby disrupting NTHI biofilms. Non-typeable Haemophilus influenzae biofilms were grown in supplemented BHI media in 8 well chambered coverglass slides at 37° C. and 5% CO₂. After 24 h, biofilms were treated as indicated. (FIG. 38A) After 40 h, biofilms were washed, probed with primary antibodies directed against DNABII proteins (light gray dots), a fluorescent secondary antibody, stained with DAPI (gray regions), and visualized via fluorescence microscopy. (FIG. 38B) Media from biofilm cultures was collected and analyzed via Western blot with a primary antibody that recognizes DNABII proteins. HMGB1 treatment displaced DNABII proteins from the biofilm matrix. (FIG. 38C) COMSTAT quantification NTHI biofilms stained with LIVE/DEAD® and visualized with confocal microscopy after treatment with 5 mg/mL HMGB1. HMGB1 disrupts biofilms by displacing DNABII proteins.

FIGS. 39A-39B: HMGB1 promotes biofilm resolution in an experimental model (chinchilla host) of OM. Diluent or 5 μg rHMGB1 or mHMGB1 were delivered directly to middle ears of chinchillas at 4 and 5 days post infection with NTHI. Animals were sacrificed 24 h later, and their middle ears were imaged (FIG. 39A) and blindly scored (FIG. 39B) based on the criteria described in the bottom of (FIG. 39A). Bars represent SEM. ***P<0.001. Images and scoring demonstrate HMGB1 promoted clearance of pre-existing NTHI biofilms in situ.

FIGS. 40A-40C: HMGB1 disrupts Burkholderia cenocepacia biofilms. (FIG. 40A) B. cenocepacia biofilms were grown in LB media in 8 well chambered coverglass slides at 37° C. and 5% CO₂. After 24 h, biofilms were treated 5 mg/mL HMGB1. After a total of 40 h, biofilms were stained with LIVE/DEAD®, visualized via CLSM and analyzed by COMSTAT. Bars represent the SEM. (FIG. 40B) C57BL/6 mice were infected with 10⁷ CFU i.t., and simultaneously received 5 mg rHMGB1 or mHMGB1, a C45 S non-inflammatory variant. Aggregates of B. cenocepacia were visible by fluorescence microscopy in sections probed with an α-B. cenocepacia antibody (gray). After 18 h, (FIG. 40C) CFUs were quantified in BAL. Bars represent SD. *P<0.05, ***P<0.001. HMGB1 treatment significantly disrupts B. cenocepacia biofilms in situ.

FIG. 41: HMGB1 reverts polyamine-induced Z-DNA to a nuclease sensitive B-DNA state. A poly(dG-dC) substrate was incubated with spermidine and HMGB1. Degradation products were visualized by gel electrophoresis. HMGB1 treatment restored nuclease sensitivity to spermidine-induced Z-DNA substrates.

FIGS. 42A-42D: Both DNABII proteins and polyamines are required to rescue cation exchanger (P11)-mediated biofilm prevention. (FIG. 42A) NTHI biofilm growth was initiated at 37° C., 5% CO₂ in the basolateral chamber of a transwell plate system whereas P11 resin was added to the apical chamber, and incubated for 16 h. (FIG. 42B) Biofilms were stained, fixed, visualized via CLSM, and analyzed by COMSTAT. Bars represent the SEM. Statistical significance compared to Control (no P11) was assessed by unpaired t-tests, ****P<0.0001. (FIG. 42C) Spermidine (Spd, 300 μM) and HU (500 nM) were added at seeding. Biofilms were quantified as in (FIG. 42B). **P<0.01, ***P<0.001, and ****P<0.0001 compared to Control (no P11). (FIG. 42D) Representative images of biofilms formed in the conditions quantified in (FIG. 42C), average biomass in top right. P11 addition prevents biofilm formation in a dose-dependent manner. Exogenous spermidine and HU together restore biofilm development but not either alone, which suggests that P11 anti-biofilm activity is the result of titration of structural components (polyamines, DNABII proteins) away from the biofilm EPS without direct contact.

FIGS. 43A-43B: Specificity of B- and Z-DNA antibodies. (FIG. 43A) Brominated genomic DNA (2 μg/mL) and polydGdC was incubated in buffer and the absorbance values at 260 nm and 295 nm were measured and the A260/295 ratio was calculated. A ratio >8.6 is indicative of B-DNA (dark gray), while a value near 3.2 is indicative of Z-DNA (white). (FIG. 43B) An ELISA plate was coated with 1 μg of poly dGdC (B-DNA) or brominated poly dGdC (Z-DNA Hindler at al. (2013) J Clin Microbiol. 51(6):1678-84.), followed by blocking with 0.5% BSA. Wells were then probed with mouse (ms) IgG1 (neg. control), mouse anti-B-DNA, or mouse anti-Z-DNA and detected with a secondary goat anti-mouse IgG-HRP. TMB (3,3′,5,5′-tetramethylbenzidine) was the colorimetric substrate used for HRP detection (dark gray wells). The anti-B and anti-Z antibodies were specific for their respective DNA forms.

FIG. 44: DNABII proteins, polyamines, and eDNA (B- and Z-DNA) steadily accumulate within the EPS of NTHI biofilms over time. Immunofluorescence CLSM images of NTHI biofilms at various stages of formation, probed with anti-B-DNA antibodies (light gray; first from the left), anti-anti-spermidine (light gray; second from the left), anti-DNABII (gray; third from the left), or anti-Z-DNA (white; fourth from the elft). Mature biofilms incorporate an increasing amount of each TEDS component within the EPS over time.

FIG. 45: Polyamines and DNABII proteins stimulate Z-DNA, which is DNAse resistant. Immunofluorescence images of EPS scaffold mimetic formed de novo by addition of DNABII protein (HU_{NTHI}, 500 nM) and spermidine (300 μM) to purified genomic DNA (2 μg/ml) and incubation at 37° C. for 16 h. The biofilm scaffold mimetics were incubated with Pulmozyme® (DNase, 5 U/ml) for 1 h and then probed for B-DNA (dark gray) and Z-DNA (white). Scale bar 10 μM. B-DNA and Z-DNA were observed within the mimetic structures, and DNase addition selectively eliminated B-DNA whereas Z-DNA remained intact. Thus, polyamines and DNABII proteins can induce the DNase-resistant state by conversion of eDNA from B-DNA to Z-DNA.

FIG. 46: TEDS and Z-DNA are present within the biofilm EPS formed by multiple human pathogens. (A) Top: Immunofluorescence CLSM images of indicated biofilms probed with naive or anti-HU (gray) and anti-spermidine (Spd, light gray) antibodies, co-localization is white. Bottom: Immunofluorescence CLSM images of biofilms formed for 40 h of the indicated bacteria, probed with naive or anti-B-DNA (dark gray) and anti-Z-DNA antibodies (white). A well-characterized, Z-DNA-specific monoclonal antibody (clone Z22 Heydorn et al. (2002) COMSTAT. Microbiology; 146; Yang et al. (2017) Paediatr Respir Rev. 21:65-7; Xu et al. (2016) Molecules; 21(8).) was used to detect Z-DNA. DNABII, polyamines, Z-DNA and B-DNA components are all present in the EPS of multiple bacterial biofilms. Z-DNA is an integral part of the EPS of multiple bacterial biofilms at different steady state levels.

FIGS. 47A-47B: Z-DNA is present in in vivo and ex vivo samples. Left: Z-DNA (white) and B-DNA (dark gray) within (FIG. 47A) NTHI biofilm during experimental NTHI-induced OM and (FIG. 47B) sputum from patient with cystic fibrosis. Inset, neg. control. Scale bar 10 μm. Right: DNABII protein (gray) and spermidine (Spd, light gray) within (FIG. 47A) NTHI biofilm during experimental NTHI-induced OM and (FIG. 47B) sputum from patient with cystic fibrosis. Inset, neg. control. Scale bar 10 μm. DNABII, polyamines, Z-DNA and B-DNA components are all present within sections of the chinchilla middle ear infected with NTHI biofilms. Z-DNA is an integral part of the EPS of multiple bacterial biofilms at different steady state levels.

FIGS. 48A-48B: Z-DNA in biofilms forms a nuclease-resistant scaffold. (FIG. 48A) Biofilms formed for 24 h by the indicated bacteria (Kp=K. pneumoniae) were incubated with DNase (Pulmozyme®; 40 U/ml) for an additional 16 h. Biofilms were then probed with anti-B-DNA (dark gray) and anti-Z-DNA (white) antibodies and counterstained with the bacterial cell membrane stain FM4-64™ (not shown). (FIG. 48B) Changes in abundance of B-DNA or Z-DNA were quantified using ImageJ by calculation of the ratio of fluorescence intensity of B-DNA or Z-DNA after DNase addition divided by the fluorescence intensity in the absence of DNase (Control). Fluorescence intensity was normalized to the fluorescence signal of FM4-64™-stained cells. Bars represent the SEM. Statistical significance compared to Control (no DNase, dotted line) was assessed by paired t-tests, *P<0.05; ****P<0.0001. While B-DNA is degraded by DNase, Z-DNA is resistant and serves to maintain the structural integrity of the biofilm EPS.

FIG. 49: Z-DNA stabilization stimulates biofilm formation. NTHI and UPEC biofilm growth was initiated in the presence of naive or anti-Z-DNA antibodies (5 mg/ml) for 16 h. Biofilms were stained, fixed, visualized via CLSM, and analyzed by COMSTAT. Bars represent the SEM. Statistical significance compared to Control (no antibodies) was assessed by paired t-tests, *P<0.05, **P<0.01. Anti-Z-DNA antibodies stimulate biofilm formation in a dose-dependent manner, whereas naïve antibodies do not.

FIGS. 50A-50B: Equilibrium shift of B-DNA to Z-DNA conversion alters biofilm formation. NTHI biofilms formed for 24 h were incubated with cerium chloride (CeCl₃) or chloroquine for an additional 16 h and were probed with naive or anti-Z-DNA (white) by immunofluorescence CLSM. (FIG. 50A) Representative image of NTHI biofilm indicated an increased Z-DNA abundance upon addition of 1 mM CeCl₃. (FIG. 50B) Representative image of NTHI biofilm indicated a decreased Z-DNA abundance upon addition of 1 μM chloroquine.

FIGS. 51A-51B: Equilibrium shift of B-DNA to Z-DNA conversion alters biofilm formation. NTHI biofilms formed for 24 h were incubated with cerium chloride (CeCl₃) (FIG. 51A) or chloroquine (FIG. 51B) for an additional 16 h. Z-DNA was measured by IF and CLSM using anti-Z-DNA and cells were stained with the membrane stain FM4-64. Bars represent the SEM. Statistical significance compared to control was assessed by paired t-tests, *P<0.05, **P<0.01. CeCl₃ increased Z-DNA and biofilm formation, whereas chloroquine reduced Z-DNA and biofilm development.

FIGS. 52A-52B: RNA homeostasis modulates bacterial biofilm development. (FIG. 52A) NTHI biofilm growth was initiated in the presence of RNase A for 16 h. Biofilms were stained, fixed, visualized via CLSM, and analyzed by COMSTAT. Addition of RNase A stimulated biofilm formation in a dose-dependent manner, likely by the release of polyamines, a critical catalyst for the extracellular conversion of B-DNA to Z-DNA. (FIG. 52B) NTHI biofilms formed for 16 h were incubated with tRNA and analyzed as in (FIG. 52A). Guanosine monophosphate (GMP) served as a negative control. Bars represent the SEM. Statistical significance compared to Control (no RNase A or tRNA/GMP) was assessed by paired t-tests, *P<0.05, **P<0.01. Addition of tRNA (but not GMP) disrupted early NTHI biofilms in a dose-dependent manner, likely by sequestration of polyamines from the biofilm EPS.

FIG. 53: DNABII and polyamines synergize to covert B-DNA to Z-DNA. In one aspect, the agents are administered in the absence of a DNAse. Left: Genomic DNA (2 μg/mL) was incubated with spermidine (spd:300 μM), HU (500 nM), a combination of both spd and HU for 2 h at 37° C. Incubation of gDNA with 3.6M NaCl was used as a positive Z-DNA control. Middle: gDNA (2 μg/mL) was incubated with increased concentrations of Cerium chloride (CeCl₃) which is known to induce Z-DNA. Right: poly(dGdC) (1 μg) was incubated with either NaCl (3.6 M), chloroquine (100 μM), or a combination of both NaCl and chloroquine. Chloroquine prevents the transition of B-DNA to Z-DNA. The absorbance values at 260 nm and 295 nm were measured and the A260/295 ratio was calculated. A ratio >8.6 is indicative of B-DNA, while a value near 3.2 is indicative of Z-DNA. DNABII and polyamines as well as CeCl₃ synergized to induce Z-DNA, whereas chloroquine prevented Z-DNA conversion in accordance with a well-established and verified spectroscopic absorbance ratio assay.

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

A “biofilm” intends an organized community of microorganisms that at times adhere to the surface of a structure, that may be organic or inorganic, together with the polymers such as DNA that they secrete and/or release. The biofilms are very resistant to microbiotics and antimicrobial agents. They live on gingival tissues, teeth and restorations, causing caries and periodontal disease, also known as periodontal plaque disease. They also cause chronic middle ear infections. Biofilms can also form on the surface of dental implants, stents, catheter lines and contact lenses. They grow on pacemakers, heart valve replacements, artificial joints and other surgical implants. The Centers for Disease Control) estimate that over 65% of nosocomial (hospital-acquired) infections are caused by biofilms. They cause chronic vaginal infections and lead to life-threatening systemic infections in people with hobbled immune systems. Biofilms also are involved in numerous diseases. For instance, cystic fibrosis patients have Pseudomonas infections that often result in antibiotic resistant biofilms.

The term “inhibiting, competing or titrating” intends a reduction in the formation of the DNA/protein matrix that is a component of a microbial biofilm.

A “DNABII polypeptide or protein” intends a DNA binding protein or polypeptide that is composed of DNA-binding domains and thus have a specific or general affinity for microbial DNA. In one aspect, they bind DNA in the minor grove. Non-limiting examples of DNABII proteins are an integration host factor (IHF) protein and a histone-like protein from E. coli strain U93 (HU). Other DNA binding proteins that may be associated with the biofilm include DPS (Genbank Accession No.: CAA49169), H-NS (Genbank Accession No.: CAA47740), Hfq (Genbank Accession No.: ACE63256), CbpA (Genbank Accession No.: BAA03950) and CbpB (Genbank Accession No.: NP_418813).

An “integration host factor” of “IHF” protein is a bacterial protein that is used by bacteriophages to incorporate their DNA into the host bacteria. They also bind extracellular microbial DNA. The genes that encode the IHF protein subunits in E. coli are himA (Genbank Accession No.: POA6X7.1) and himD (POA6Y1.1) genes. Homologs for these genes are found in other organisms, and peptides corresponding to these genes from other organisms are disclosed in the art, for example in Table 10 of U.S. Pat. No. 8,999,291.

“HMGB1” is an high mobility group box (HMGB) 1 protein that is reported to bind to and distort the minor groove of DNA and is an example of an agent. Recombinant or isolated protein and polypeptide are commercially available from Atgenglobal, ProSpecBio, Protein1 and Abnova. HMGB1 is a small protein of 215 amino acid protein (of approx 30 Kda) composed of 3 domains: two positively charged domains the A and B box each one comprising of 80 amino acids and a negatively charged carbocyl terminus the acidic C tail which consists of approximately 30 consecutive aspartate and glutamate residues. Provided below is a non-limiting example of a polypeptide sequence of the wildtype HMGB1:

MGKGDPKKPRRKMSSYAFFVQTCREEHKKKHPDASVNFSEFSKKCSERWK
TMSAKEKGKFEDMAKADKARYEREMKTYI_PPKGETKKKF_KDPNAPKRP
PSAFFLFCSEYRPKIKGEHPGLSIGDVAKKLGEMWNNTAADDKQPYEKKA
EKLKEKYEKDIAAYRAKGKPDAAKKGVV KAEKSKKKKEEEEGEEDEEDE
EEEEDEEDEDEEEDDDDE

Bolded amino acids (amino acids 1-70) depict the A Box domain.

The italicized amino acids (amino acids 88-164) depict the B Box domain.

The underlined amino acids (amino acids 186-215) depict the C-tail domain. These are non-limiting examples of fragments, e.g., the A Box domain, the B Box domain, the A and B box domains (AB box domain) the C-tail domain and the N-domain (amino acids 1-185). In one aspect, the fragment consists essentially of the C-terminal domain or a polypeptide comprising the B Box domain.

“HU” or “histone-like protein from E. coli strain U93” refers to a class of heterodimeric proteins typically associate with E. coli. HU proteins are known to bind DNA junctions. Related proteins have been isolated from other microorganisms. The complete amino acid sequence of E. coli HU was reported by Laine et al. (1980) Eur. J. Biochem 103(3)447-481. Antibodies to the HU protein are commercially available from Abeam.

The term “surface antigens” or “surface proteins” refers to proteins or peptides on the surface of cells such as bacterial cells. Examples of surface antigens are Outer membrane proteins such as OMP P5 (Genbank Accession No.: YP_004139079.1), OMP P2 (Genbank Accession No.: ZZX87199.1), OMP P26 (Genbank Accession No.: YP_665091.1), rsPilA or recombinant soluble PilA (Genbank Accession No.: EFU96734.1) and Type IV Pilin (Genbank Accession No.: Yp_003864351.1).

The term “Haemophilus influenzae” refers to pathogenic bacteria that can cause many different infections such as, for example, ear infections, eye infections, and sinusitis. Many different strains of Haemophilus influenzae have been isolated and have an IhfA gene or protein. Some non-limiting examples of different strains of Haemophilus influenzae include Rd KW20, 86-028NP, R2866, PittGG, PittEE, R2846, and 2019.

“Microbial DNA” intends single or double stranded DNA from a microorganism that produces a biofilm.

“Inhibiting, preventing or breaking down” a biofilm intends the prophylactic or therapeutic reduction in the structure of a biofilm.

A “bent polynucleotide” intends a double strand polynucleotide that contains a small loop on one strand which does not pair with the other strand. In some embodiments, the loop is from 1 base to about 20 bases long, or alternatively from 2 bases to about 15 bases long, or alternatively from about 3 bases to about 12 bases long, or alternatively from about 4 bases to about 10 bases long, or alternatively has about 4, 5, or 6, or 7, or 8, or 9, or 10 bases.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets. The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen tinder a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address: align.genome.jp, last accessed on Mar. 7, 2011.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the terms “treating,” “treatment” and the like are used herein to mean obtaining a desired pharmacologic and/or physiologic effect. The effect may be therapeutic in terms of a partial or complete cure for a disorder and/or adverse effect attributable to the disorder. As used herein, “treating” or “treatment” of a disease in a subject can also refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis. When the disease is SLE (systemic lupus erythematosus) and/or cystic fibrosis (CF), evidence of treatment included reduced evidence of inflammation, and/or the level of autoimmune activity or symptoms.

To prevent intends to prevent a disorder or effect in vitro or in vivo in a system or subject that is predisposed to the disorder or effect. An example of such is preventing the formation of a biofilm in a system that is infected with a microorganism known to produce one.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

A “biologically active agent” or an active agent disclosed herein intends one or more of an isolated or recombinant polypeptide, an isolated or recombinant polynucleotide, a vector, an isolated host cell, or an antibody, as well as compositions comprising one or more of same.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

A “peptide conjugate” refers to the association by covalent or non-covalent bonding of one or more polypeptides and another chemical or biological compound. In a non-limiting example, the “conjugation” of a polypeptide with a chemical compound results in improved stability or efficacy of the polypeptide for its intended purpose. In one embodiment, a peptide is conjugated to a carrier, wherein the carrier is a liposome, a micelle, or a pharmaceutically acceptable polymer.

“Liposomes” are microscopic vesicles consisting of concentric lipid bilayers. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloteoylphosphatidylcholine (POPC), palmitoyloleoylphosphatidylethanolamine (POPE) and dioleoylphosphatidylethanolamine 4-(N-maleimido-triethyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioteoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs). The biological active agents can be encapsulated in such for administration in accordance with the methods described herein.

A “micelle” is an aggregate of surfactant molecules dispersed in a liquid colloid. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the head groups at the center with the tails extending out (water-in-oil micelle). Micelles can be used to attach a polynucleotide, polypeptide, antibody or composition described herein to facilitate efficient delivery to the target cell or tissue.

The phrase “pharmaceutically acceptable polymer” refers to the group of compounds which can be conjugated to one or more polypeptides described here. It is contemplated that the conjugation of a polymer to the polypeptide is capable of extending the half-life of the polypeptide in vivo and in vitro. Non-limiting examples include polyethylene glycols, polyvinylpyrrolidones, polyvinylalcohols, cellulose derivatives, polyacrylates, polymethacrylates, sugars, polyols and mixtures thereof. The biological active agents can be conjugated to a pharmaceutically acceptable polymer for administration in accordance with the methods described herein.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein the term “eDNA” refers to extracellular DNA found as a component to pathogenic biofilms.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., PCT International Application Publication No. WO 95/27071. Ads do not require integration into the host cell genome. Recombinant Ad derived vectors, particularly those that reduce the potential for recombination and generation of wild-type virus, have also been constructed. See, PCT International Application Publication Nos. WO 95/00655 and WO 95/11984, Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)2, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-DNABII, anti-IHF, anti-HU, anti-OMP P5, for example, refers to a monoclonal or polyclonal antibody that binds and/or has an affinity to a particular protein. For example, “anti-IHF” refers to an antibody that binds to the IHF protein. The specific antibody may have affinity or bind to proteins other than the protein it was raised against. For example, anti-IHF, while specifically raised against the IHF protein, may also bind other proteins that are related either through sequence homology or through structure homology.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

The terms “antigen” and “antigenic” refer to molecules with the capacity to be recognized by an antibody or otherwise act as a member of an antibody-ligand pair. “Specific binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

An “altered antigen” is one having a primary sequence that is different from that of the corresponding wild-type antigen. Altered antigens can be made by synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand. (Ferguson et al. (1988) Ann. Rev. Biochem. 57:285-320). A synthetic or altered antigen disclosed herein is intended to bind to the same TCR as the natural epitope.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

The term “passive immunity” refers to the transfer of immunity from one subject to another through the transfer of antibodies. Passive immunity may occur naturally, as when maternal antibodies are transferred to a fetus. Passive immunity may also occur artificially as when antibody compositions are administered to non-immune subjects. Antibody donors and recipients may be human or non-human subjects. Antibodies may be polyclonal or monoclonal, may be generated in vitro or in vivo, and may be purified, partially purified, or unpurified depending on the embodiment. In some embodiments described herein, passive immunity is conferred on a subject in need thereof through the administration of antibodies or antigen binding fragments that specifically recognize or bind to a particular antigen. In some embodiments, passive immunity is conferred through the administration of an isolated or recombinant polynucleotide encoding an antibody or antigen binding fragment that specifically recognizes or binds to a particular antigen.

In the context of this disclosure, a “ligand” is a polypeptide. In one aspect, the term “ligand” as used herein refers to any molecule that binds to a specific site on another molecule. In other words, the ligand confers the specificity of the protein in a reaction with an immune effector cell or an antibody to a protein or DNA to a protein. In one aspect it is the ligand site within the protein that combines directly with the complementary binding site on the immune effector cell.

As used herein, the term “inducing an immune response in a subject” is a term well understood in the art and intends that an increase of at least about 2-fold, at least about 5-fold, at least about 10-fold, at least about 100-fold, at least about 500-fold, or at least about 1000-fold or more in an immune response to an antigen (or epitope) can be detected or measured, after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, Calif.).

An example of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

The term “modulate an immune response” includes inducing (increasing, eliciting) an immune response; and reducing (suppressing) an immune response. An immunomodulatory method (or protocol) is one that modulates an immune response in a subject.

### Modes for Carrying Out the Disclosure

### I. The Biofilm Structure and Disease

The reservoir of bacteria that sustain chronic and recurrent bacterial infections reside in a biofilm, a community of bacteria that have adhered to a surface and, when in this state, can resist clearance by the host immune system as well as by antimicrobials. Indeed, bacteria in a biofilm state are typically >1000-fold more resistant to antibiotics than the same bacteria in a free-living or planktonic state. Ceri et al. (1999) J Clin Microbiol. 37(6):1771-6. The ability of biofilm bacteria to resist clearance is owed mostly to the semi-permeable self-made matrix or extracellular polymeric substances (EPS) that acts both as a physical barrier to environmental hazards, as well as creates conditions for an altered physiology that limits metabolism to enhance this resistant state. While the constituents of the EPS are specific to each bacterium and include proteins, polysaccharides, lipids and nucleic acids, the nature of the EPS needs be sufficiently conducive for bacterial genera at large to interact productively (e.g. as metabolic partners). To this end, several recent discoveries have led to the possibility of an underlying universal EPS structure common to all eubacteria. Whitchurch and colleagues (Whitchurch et al. (2002) Science. 295(5559)) showed that extracellular DNA (eDNA) was a common EPS constituent and that treatment of bacteria with DNase was sufficient to prevent biofilm formation. While this result was replicated for multiple genera, the use of DNase failed to treat extant biofilms greater than a day or two after biofilm seeding despite the fact that eDNA is evident in biofilms throughout their lifecycle. Separately, Applicants previously identified the DNABII proteins, the only family of nucleoid associated proteins (NAPs) that is common to all eubacteria, as being a necessary component of the eDNA dependent EPS. Indeed, antibodies directed against the DNABII proteins titrate DNABII proteins from the bulk medium and thereby shift the equilibrium of DNABII proteins from the eDNA-bound state to the unbound state, which results in catastrophic collapse of all bacterial biofilms tested to date, and includes mixed-species biofilms. Goodman et al. (2011) Mucosal Immunol. 4(6):625-37; Novotny et al. (2013) PLoS One. 8(6):e67629; Devaraj et al. (2015) Mol Microbiol. 96(6):1119-35; Rocco et al. (2017) Mol Oral Microbiol. 32(2):118-30; Gustave et al. (2013) J Cyst Fibros. 12(4):384-9; Novotny et al. (2016) EBioMedicine. 10:33-44. Importantly, unlike DNase, treatment with antibody directed against DNABII proteins is effective at all stages of biofilm development which demonstrates that the eDNA-dependent EPS is a critical structure regardless of biofilm age. Brockson et al. (2014) Mol Microbiol. 93(6):1246-58. Despite, knowing that eDNA and members of the DNABII family are essential components of the EPS, understanding the complete EPS structure has proved elusive; DNABII proteins and DNA are insufficient to recapitulate the functional EPS structures in vitro. Applicants describe herein that for multiple human pathogens that as the biofilm matures, the eDNA dependent EPS is dependent on both the DNABII proteins as well as polyamines such that the eDNA shifts from a B-DNA to a Z-DNA conformation. This latter result is particularly intriguing, as it likely explains the failure of DNase to disrupt mature biofilms; nucleases only cleave the more classical B-form of DNA.

### Intracellular Bacterial Nucleoid

The DNA inside bacteria is highly structured and facilitates the regulation of all forms of nucleic acid processes that include DNA replication, repair, transcription, and recombination. Unlike eukaryotic cells, bacteria are devoid of histones. Instead bacterial DNA is structured in part by a class of proteins called nucleoid associated proteins (NAPs). NAPs collectively bind DNA to create functional structures. Dillon et al. (2010) Nat Rev Microbiol. 8(3):185-95. Among the multiple NAP members that exist across genera, only the DNABII family is ubiquitous amongst all eubacteria. Dey et al. (2017) Mol Phylogenet Evol. 107:356-66. The DNABII family of proteins functions as dimers (homodimers or heterodimers depending on the species) and includes the histone-like proteins HU and IHF. HU weakly and non-specifically binds to and bends double-stranded DNA (dsDNA) but has a much higher affinity for pre-bent or structured dsDNA3. IHF like HU binds and bends DNA with a strong preference for pre-bent/structured DNA. Unlike HU, IHF is only expressed by proteobacteria and also has preference for a specific DNA consensus sequence. Swinger et al. (2004) Curr Opin Struct Biol. 14(1):28-35.

### Extracellular Bacterial Nucleoi

Extracellular DNA (eDNA) has been known to have a biological role since the discovery that the ‘transforming principle’ was the result of DNA. Avery et al. (1944) J Exp Med. 79(2):137-58. Indeed, eDNA is also critical to the extracellular matrix (extracellular polymeric substances, EPS) of bacterial biofilms. Gunn et al. (2016) J Biol Chem. 291(24):12538-46. However, the structure of biofilm eDNA, and the importance of that structure for eDNA function has thus far not been investigated.

### Chronic and Recurrent Infections are the Result of Bacterial Biofilms

While biofilms are further distinguished from planktonic bacteria by intercellular communication and transport systems, their most distinctive feature is their self-made EPS that protects the resident biofilm bacteria by both acting as a semi-permeable barrier and by creating an environment for altered/slowed metabolism; indeed biofilm bacteria are greater than 1000-fold more resistant to antibiotics than their planktonic counterparts. Ceri et al. (1999) J Clin Microbiol. 37(6):1771-6. Interestingly, the EPS of each bacterium is distinct and consists of a variety of proteins, lipids, polysaccharides, and nucleic acids. Gunn et al. (2016) J Biol Chem. 291(24):12538-46. However, while biofilms can consist of a single species, commonly in chronic infections and invariably in the environment they are comprised of multiple genera, and as such need to be able to interact productively (e.g. co-aggregation with specific metabolic partners Stacy et al. (2016) Nat Rev Microbiol. 14(2):93-105; Wolcott et al. (2013) Clin Microbiol Infect. 19(2):107-12). This community concept implies that despite varying EPS composition, each EPS must be sufficiently accommodating to allow divergent bacteria to interact within the biofilm and further, suggests that biofilm EPS likely have a universal underlying structure.

eDNA Dependent EPS has the Qualities of a Universal Underlying Architecture

Multiple groups have examined the eDNA associated with bacterial biofilms from both human and ecological genera and observed a scaffold structure (FIG. 1A). Jurcisek et al. (2017) Proc Natl Acad Sci USA. 114(32):E6632-E41; Sena-Velez et al. (2016) PLoS One. 11(6):e0156695; Wang et al. (2015) Environ Microbiol Rep. 7(2):330-40. Whitchurch and colleagues first showed that P. aeruginosa biofilms could be prevented by deoxynuclease I (DNase) treatment (Whitchurch et al. (2002) Science. 295(5559)), which indicates that eDNA is a critical structural component of the EPS. While DNase can inhibit early biofilm formation in many genera (Frederiksen et al. (2006) Acta Paediatr. 95(9):1070-4; Martins et al. (2012) Mycoses. 55(1):80-5; Hymes et al. (2013) J Infect Dis. 207(10):1491-7)), biofilms become recalcitrant to DNase over time despite the fact that eDNA clearly persists as the biofilm matures. Goodman et al. (2011) Mucosal Immunol. 4(6):625-37; Hall-Stoodley et al. (2008) BMC Microbiol. 8:173; Izano et al. (2009) Microb Pathog. 46(4):207-13; Kaplan et al. (2012) J Antibiot (Tokyo). 65(2):73-7; Novotny et al. (2013) PLoS One. 8(6):e67629; Tetz et al. (2010) DNA Cell Biol. 29(8):399-405. While this outcome was often interpreted to mean that eDNA is no longer important to the structural integrity of the EPS, Applicants have shown that not only does the eDNA persist, but it becomes the primary underlying EPS structure. Goodman et al. (2011) Mucosal Immunol. 4(6):625-37; Novotny et al. (2013) PLoS One. 8(6):e67629; Devaraj et al. (2015) Mol Microbiol. 96(6):1119-35; Rocco et al. (2017) Mol Oral Microbiol. 32(2):118-30; Brockson et al. (2014) Mol Microbiol. 93(6):1246-58.

The DNABII Family of Proteins is the Linchpin that Maintains the Structural Integrity of the Biofilm eDNA-Scaffolded EPS

Applicants previously have shown that the ubiquitous DNABII proteins, and likely no other NAPs (Devaraj et al. (2017) Microbiologyopen.), are structural constituents of the eDNA and that once removed, the eDNA structure is disrupted. Goodman et al. (2011) Mucosal Immunol. 4(6):625-37; Novotny et al. (2013) PLoS One. 8(6):e67629; Devaraj et al. (2015) Mol Microbiol. 96(6):1119-35; Rocco et al. (2017) Mol Oral Microbiol. 32(2):118-30. Indeed, the DNABII proteins were found to bind specifically to the vertices (pre-bent DNA) of the eDNA scaffold of biofilms formed in vivo whereas antibodies directed against the DNABII proteins are sufficient to undermine the structure of the eDNA-scaffolded EPS and as a result cause catastrophic collapse of both single and multi-species biofilms for every species Applicants have examined (Goodman et al. (2011) Mucosal Immunol. 4(6):625-37; Novotny et al. (2013) PLoS One. 8(6):e67629; Devaraj et al. (2015) Mol Microbiol. 96(6):1119-35; Rocco et al. (2017) Mol Oral Microbiol. 32(2):118-30), regardless of biofilm maturity. Brockson et al. (2014) Mol Microbiol. 93(6):1246-58. This disruption releases resident bacteria into a planktonic and thus antimicrobial and immune sensitive state (Goodman et al. (2011) Mucosal Immunol. 4(6):625-37; Novotny et al. (2013) PLoS One. 8(6):e67629; Brockson et al. (2014) Mol Microbiol. 93(6):1246-58), which demonstrates the importance and universality of this family of proteins in biofilm structure. Given the known interactions of the DNABII family with DNA, it was unexpected that Applicants were unable to create conditions with just DNA and DNABII proteins that recapitulate the 3-dimensional scaffoldlike structures observed in bacterial biofilms.

Polyamines are Ubiquitous Intra- and Extra-Cellularly and are Required for the eDNA-Scaffolded EPS Structure of Biofilms

Polyamines are typically short organic molecules that contain multiple primary amines that are positively charged (basic) at neutral pH and are commonly derived by decarboxylating amino acids (FIG. 1B). Michael et al. (2016) Biochem J. 473(15):2315-29. Polyamines are ubiquitous in nature, found as high as mM concentrations both intra- and extra-cellularly (Tabor et al. (1985) Microbiol Rev. 49(1):81-99) with spermidine, spermine, and putrescine being the most abundant. While polyamines are involved in multiple processes in bacterial physiology, they are perhaps most important to the eDNA-scaffolded EPS for 5 reasons. First, they bind DNA and neutralize the negative charge of the phosphate backbone and, as a result, alter DNA structure. Bachrach et al. (2005) Curr Protein Pept Sci. 6(6):559-66; Pasini et al. (2014) Amino Acids. 46(3):595-603. Second, while polyamines tend to promote protein-DNA interactions at low concentrations (mM) they tend to interfere at higher concentrations (mM), one exception being DNABII-DNA interactions that cause DNA to form thick fibers. Sarkar et al. (2009) Biochemistry. 48(4):667-75; Sarkar et al. (2007) Nucleic Acids Res. 35(3):951 61. Third, polyamine synthesis has been found to be induced during biofilm formation with some of these effects occurring extracellularly (albeit only examined to date on the bacterial membrane). Wilton et al. (2015) Antimicrob Agents Chemother. 60(1):544-53. Fourth, atomic force microscopy (AFM) experiments to visualize mixtures of polyamines and DNA reveal a structure highly similar to the biofilm eDNA scaffolds (FIG. 1C). Finally, polyamines can induce a conversion of native B-form DNA into Z-form DNA in prone sequences at physiologic concentrations (100 Thomas et al. (1986) Nucleic Acids Res. 14(16):6721-33; Thomas et al. (1988) J Mol Biol. 201(2):463-7.

Conversion of B-DNA into Z-DNA May be a Novel Means to Render the eDNA-Scaffolded EPS Nuclease Resistant and Create a Stable Structural Material

B-DNA and Z-DNA are distinct conformations of dsDNA that exist in equilibrium, with B-DNA predominating under most physiologic conditions. Alternating purines and pyrimidines (particularly dGdC) are more prone to exist as Z-DNA in either high salt (molar mono or divalent cations) or under negative supercoiling. Pohl et al. (1983) Cold Spring Harb Symp Quant Biol. 47 Pt 1:113-7; Pohl et al. (1986) Proc Natl Acad Sci USA. 83(14):4983-7. In the latter case, regions prone to form Z-DNA can be juxtaposed next to B-DNA briefly during transcription when negative supercoiling is transiently induced. Rahmouni et al. (1992) Mol Microbiol. 6(5):569-72. Whereas B-DNA bases adopt a right-handed helix (10 bp/turn), Z-DNA forms a left-handed helix (12 bp/turn). Jovin et al. (1987) Ann Rev Phys Chem. 38:521-60. B-DNA has two grooves (major and minor), and most interacting proteins recognize/bind in the major groove due to its larger size and discriminating hydrogen bond donors and acceptors for each nucleotide base. In contrast, the major groove is absent in Z-DNA, and most of those binding contacts are found on the convex face. Jovin et al. (1987) Ann Rev Phys Chem. 38:521-60. Z-DNA possesses a single groove corresponding to the minor groove of B-DNA. Interestingly, the DNABII proteins are one of only a few DNA binding proteins that bind in the minor groove (Kim et al. (2014) Acta Crystallogr D Biol Crystallogr. 70(Pt 12):3273-89), suggesting they may bind Z-DNA. The shifting of eDNA from B-DNA to Z-DNA is consistent with 4 observations of the eDNA-scaffolded EPS. First, the shift to Z-DNA occurs under conditions present in the biofilm EPS; prone sequences will shift to Z-DNA in the presence of physiologic (100 mM) concentrations of some polyamines (spermidine and spermine). Second, Z-DNA tends to aggregate and form fibers. Chaires et al. (1988) J Biomol Struct Dyn. 5(6):1187-207. Strong negative charge neutralization (e.g. polyamines) of the phosphate backbone favors Z-DNA since the phosphates in the Z-DNA backbone are closer together than in B-DNA but is also permissive for DNA aggregation. Third, Z-DNA is stiffer than B-DNA with almost a 3-fold increase in persistence length (Thomas et al. (1983) Nucleic Acids Res. 11(6):1919-30) consistent with the straight fibers that Applicants observe in the eDNA scaffold (FIG. 1A, FIG. 2, FIG. 3). And finally, Z-DNA is nuclease resistant. Unlike canonical B-DNA binding proteins, there are only a few known proteins that recognize Z-DNA (Athanasiadis et al. (2012) Semin Cell Dev Biol. 23(3):275-80) (however, Z- vs B-DNA discriminating antibodies are available). Bergen et al. (1987) J Immunol. 139(3):743-8. Interestingly, Z-DNA binding proteins are homologous, bind Z-DNA over B-DNA with 1,000- to 10,000-fold higher affinity (Herbert et al. (1996) J Biol Chem. 271(20):11595-8), induce the Z-DNA conformation in prone sequences and, in at least the case of ZBP1, are part of the innate immune system that detects the presence of microbial DNA. Athanasiadis et al. (2012) Semin Cell Dev Biol. 23(3):275-80. Importantly, proteins that use B-DNA as a substrate (e.g. nucleases) fail to recognize and thus function on the same DNA sequence in the Z configuration despite the fact that Watson Crick base pairing is preserved.

### II. A Tripartite Approach

Applicants have previously shown that eDNA-DNABII interactions serve to maintain the structural integrity of the biofilm EPS (Goodman et al. (2011) Mucosal Immunol. 4(6):625-37; Novotny et al. (2013) PLoS One. 8(6):e67629; Devaraj et al. (2015) Mol Microbiol. 96(6):1119-35; Rocco et al. (2017) Mol Oral Microbiol. 32(2):118-30; Devaraj et al. (2017) Microbiologyopen.; Brockson et al. (2014) Mol Microbiol. 93(6):1246-58), and that disrupting these interactions leads to positive outcomes ex vivo (Gustave et al. (2013) J Cyst Fibros. 12(4):384-9) and in vivo (Goodman et al. (2011) Mucosal Immunol. 4(6):625-37; Novotny et al. (2016) EBioMedicine. 10:33-44; Freire et al. (2017) Mol Oral Microbiol. 32(1):74-88). Although it was shown that both DNA and DNABII proteins are necessary, they are not sufficient to recapitulate the EPS scaffold architecture. Disclosed herein is a tripartite eDNA-dependent scaffold (TEDS) of the eDNA-DNABII dependent EPS that relies on the presence and relative location of the (1) eDNA, (2) DNABII proteins, and the newly discovered EPS constituent, (3) polyamines.

In one aspect, Applicants show that in addition to eDNA and DNABII proteins, polyamines are an essential component of the TEDS structure of bacterial biofilms. Second, Applicants show that together, all three of these components facilitate the formation of a universal EPS that can foster productive interactions amongst bacterial genera in the protective biofilm state. Third, using these components, Applicants define and recapitulate this universal structure and provide evidence consistent with the observations of thick double stranded DNA fibers, induction of a nuclease resistant state, and demonstrate whether this state requires Z-DNA as a structural endpoint. Finally, this provides diagnostic and therapeutic interventions that focus on the TEDS structure itself as a target for intervention.

### A. Polyamines

Polyamines Function in Concert with DNABII Proteins to Direct Assembly of eDNA Scaffolds

The chinchilla model of acute otitis media caused by NTHI faithfully recapitulates the course and pathophysiology of human disease (Bakaletz et al. (2009) Expert Rev Vaccines. 8(8):1063-82) and is dependent on a recalcitrant biofilm in the middle ear. Using this model, Applicants previously showed that DNABII proteins associate with eDNA, which localize to the vertices of eDNA strands (FIG. 1A) (Goodman et al. (2011) Mucosal Immunol. 4(6):625-37) and these eDNA strands appear strikingly similar to polyamines visualized with DNA by AFM (FIG. 1C). Iacomino et al. (2011) Biomacromolecules. 12(4):1178-86. DNABII proteins bind to DNA in the presence of mM concentrations of spermidine in vitro (FIG. 2B), which led to the investigation of the potential interaction between DNABII proteins and polyamines to produce a biofilm eDNA scaffold structure. Applicants performed immunofluorescence confocal laser scanning microscopy (CLSM) on sections of fixed and embedded middle ear mucosal biofilms to visualize polyamines interacting with eDNA within the biofilm EPS as Applicants have previously observed for DNABII proteins. Immunofluorescence images show that eDNA and polyamines co-localize along fibers that are present within the mucosal biofilm (FIG. 2A) and are visually similar to those observed by Hud and coworkers with DNABII proteins and polyamines in vitro (Sarkar et al. (2009) Biochemistry. 48(4):667-75; Sarkar et al. (2007) Nucleic Acids Res. 35(3):951 61) (FIG. 2B).

### Polyamine Biosynthesis is Required for Biofilm Structure

Applicants investigated whether the broad action polyamine biosynthesis inhibitor dicyclohexylamine (DCHA) would alter NTHI biofilm biogenesis in vitro. DCHA inhibits spermidine synthase (Paulin et al. (1986) Antonie Van Leeuwenhoek. 52(6):483-90; Pegg et al. (1983) FEBS Lett. 155(2):192-6), the enzyme that catalyzes the conversion of putrescine to spermidine. Although DCHA did not affect NTHI growth (data not shown), DCHA inhibited biofilm biogenesis in vitro, decreasing average thickness and biomass as determined by COMSTAT analysis (Heydorn et al. (2000) Microbiology. 146 (Pt 10):2395-407) of CLSM images of LIVE/DEAD®-stained NTHI biofilms (FIG. 3A). Furthermore, Applicants found that DCHA inhibited production of the eDNA scaffold in early biofilm development (FIG. 3B), which suggests that polyamine biosynthesis is a required step in biofilm formation. In support of these findings, Applicants observed that DCHA reduced extracellular polyamine incorporation into the biofilm EPS by immunofluorescence (FIG. 3C), and that the defects in biofilm biogenesis could be compensated for by addition of exogenous spermidine (FIG. 3A). Together, these results reveal that polyamine incorporation into the EPS is critical for the development and function of the eDNA scaffold in supporting robust biofilm growth.

### Anti-DNABII Disrupts DNABII-Polyamine Dependent DNA Structure

Immunofluorescence was used to determine whether DNABII proteins are incorporated into EPS mimetic structures. Spermidine (300 μM) and HU (1 μM) were incubated with genomic DNA (2 μg/ml). EPS mimetic structures were then probed with naïve (control) or anti-DNABII IgGs, a fluorescent secondary antibody, stained with DAPI, and imaged by CLSM. DNABII proteins were fully incorporated into the EPS structure (FIG. 4A). Next, EPS mimetic structures were formed, treated with anti-DNABII antibodies, stained with DAPI, and imaged by CLSM. The sequestration of DNABII proteins with anti-DNABII antibodies from the EPS mimetics drastically reduced abundance and size of aggregate structures (FIG. 4B), which further confirmed DNABII incorporation and its importance for DNA structural stability.

### NTHI Biofilm Disruption by the Cation-Exchanger P11 can be Prevented by Exogenous DNABII (HU) and Spermidine Addition

Phosphocellulose (P11) is a negatively charged resin that has high affinity for positively charged molecules, such as polyamines and DNABII proteins. To determine the effect of P11 sequestration of these molecules on bacterial biofilm formation, Applicants utilized a transwell system. NTHI growth was initiated in the basolateral chamber while P11 (1% w/v) was added to the apical chamber at seeding. At 16 h, the biofilms were washed and stained with LIVE/DEAD®, imaged using CLSM, and analyzed with COMSTAT. P11 significantly reduced average thickness and biomass (FIG. 5). To determine whether P11 cation depletion included polyamines and/or DNABII proteins, NTHI growth was initiated in the basolateral chamber with the exogenous addition of 1 mM spermidine and 1 μM HU, while P11 was added at 1% w/v to the apical chamber. Both spermidine and HU were required structural components of the biofilm matrix and only together prevented biofilm disruption by P11 (FIG. 5); individually HU and spermidine were insufficient (data not shown).

### DNase is Unable to Disrupt Mature Pathogenic Bacterial Biofilms

Applicants evaluated the antibiofilm effect of Pulmozyme®, a recombinant human DNase that is used in conjunction with standard therapies for the management of cystic fibrosis (CF) patients to improve pulmonary function. Yang et al. (2017) Paediatr Respir Rev. 21:65-7. Pulmozyme® was added either at seeding (biofilm prevention) or to pre-formed biofilms (biofilm disruption) (FIG. 6). The resultant biofilms were stained with LIVE/DEAD® and evaluated using CLSM and COMSTAT analysis. Addition of DNase at seeding resulted in a significant reduction in average thickness and biomass of NTHI and UPEC biofilms compared to untreated biofilms (FIG. 6). However, DNase was unable to disrupt mature biofilms (FIG. 6) which suggests that eDNA loses susceptibility to nuclease digestion as biofilms develop. Applicants propose that as biofilms mature eDNA is either sterically protected from nucleases and/or eDNA takes on a novel structure making it recalcitrant to nuclease digestion.

### DNABII Proteins and Polyamines Interact Synergistically to Confer Nuclease Resistance to DNA

Immunofluorescence of NTHI biofilms probed with anti-DNABII and antispermidine antibodies indicated that polyamines co-localize with DNABII proteins in vitro (FIG. 7A) and in vivo (FIG. 7B). Applicants hypothesized that DNABII and polyamines interact with eDNA synergistically to confer DNase resistance to mature biofilms. To test synergism in vitro, Applicants incubated NTHI genomic DNA (gDNA) with spermidine (10, 20, 50, or 100 μM) in the presence or absence of HU (50 or 100 nM) and DNase (0.5 units). Degradation was assessed by agarose gel electrophoresis and UV illumination. While spermidine (>100 μM) and HU (1 μM) protected gDNA individually, admixed lower levels of spermidine (<50 μM) and HU (<100 nM) inhibited gDNA digestion synergistically (FIG. 7C) suggesting nuclease resistance is due to the combination of DNABII proteins and polyamines within the biofilm matrix. Next, Applicants wanted to test whether EPS mimetic structures were resistant to DNase. HU (1 μM) and spermidine (300 mM) were incubated with gDNA (2 μg/ml) for 40 h to create an EPS scaffold mimetic, followed by treatment with DNase (5 units). As shown by CLSM, these DNABII-polyamine dependent DNA structures were resistant to DNase (FIG. 7D), similar to mature biofilms.

### B. DNABII, Polyamines, and DNA Form Z-DNA In Vitro

Polyamines cause Z-DNA prone sequences to shift the B-Z equilibrium into the Z-DNA configuration upon binding (Thomas et al. (1986) Nucleic Acids Res. 14(16):6721-33; Thomas et al. (1988) J Mol Biol. 201(2):463-7), while DNABII proteins bend and condense DNA. Due to the synergism of inhibition by HU and spermidine binding in Applicants' in vitro DNase degradation assays (FIG. 7), and the ability of Z-DNA to resist DNase, Applicants hypothesized that the EPS mimetic structures would contain Z-DNA. EPS structures were formed as in FIG. 4 & FIG. 7 for 16 h with gDNA. Immunofluorescence CLSM was conducted probing with anti-Z-DNA antibodies, which confirmed the presence of Z-DNA (note the white color; FIG. 8A). To determine whether DNABII proteins influence the B-Z equilibrium Applicants used circular dichroism (CD). Admixtures of HU and poly (dGdC) DNA substrate exhibits inversion of the B-DNA alone ellipticity peaks (250 and 280 nm) (Jang et al. (2015) Sci Rep. 5:9943), similar to a characteristic Z-DNA spectrum (FIG. 8B).

### EPS in Bacterial Biofilms Contain Z-DNA and Polyamines

To further characterize the association of Z-DNA and polyamines, Applicants performed immunofluorescence on 40 h biofilms. Biofilms of the indicated bacterial pathogens were imaged by CLSM after probing with anti-DNABII and anti-spermidine, or anti-Z-DNA antibodies; while Z-DNA was detected within the biofilm EPS of each of the bacterial pathogens (FIG. 9) there was clearly a hierarchy between species (UPEC=Staphylococcus epidermidis<K. pneumoniae<NTHI). In all cases, there was extensive co-localization between DNABII (HU) and spermidine (note the white color), as well as a correspondence to Z-DNA abundance. Applicants further examined NTHI and UPEC biofilms at various stages of biofilm formation (24, 40, and 90 h) and observed that spermidine and Z-DNA increased concomitantly within the biofilm EPS with the age of the biofilm (FIG. 10). These data suggest that Z-DNA and spermidine are indeed an integral part of the biofilm EPS and likely contribute to its structure and DNase-resistant state.

HU Deficient NTHI Fail to Form Native Biofilms, Incorporate Polyamines, or Induce a Shift from B- to Z-DNA

Since polyamines co-localize with HU within the NTHI biofilm EPS in vitro (FIGS. 7A & FIG. 9) and in vivo (FIG. 7B), Applicants evaluated the role of HU in the incorporation of polyamines and Z-DNA within the NTHI biofilm EPS. HU deficient NTHI (hupA null; DHU) was compared to WT NTHI biofilms for the presence of polyamines and Z-DNA via immunofluorescence. The lack of HU resulted in significant reduction in polyamines and Z-DNA within the biofilm EPS compared to WT (FIG. 11) which suggests that HU is required for the presence of polyamines and Z-DNA within the NTHI biofilm EPS.

### III. Diagnostic and Therapeutic Methods

Provided herein are methods for treating a biofilm in a subject, comprising, or alternatively consisting essentially of, or yet further consisting of administering to the subject infected with a biofilm an effective amount of an agent that interferes with the binding of a polyamine to the DNA in the biofilm. In one aspect, the agent is not an HMGB1 protein, fragment or an equivalent of each thereof. In another aspect, the agent is provided in the absence of a DNAse. In a further aspect, DNAse is administered subsequent to administration of the agent. In one particulate aspect, the DNAse administered is Pulmozyme. In one aspect, the methods for treating a biofilm in a subject, comprise, or alternatively consist essentially of, or yet further consist of administering to the subject infected with a biofilm an effective amount of one or more agents that interfere with the binding of a polyamine to the DNA in the biofilm. In one aspect, the agent is not an HMGB1 protein, fragment or an equivalent of each thereof. In another aspect, the agent is provided in the absence of a DNAse. In a further aspect, DNAse is administered subsequent to administration of the agent. In one particulate aspect, the DNAse administered is Pulmozyme. In another aspect, the methods for treating a biofilm in a subject, comprise, or alternatively consist essentially of, or yet further consist of administering to the subject infected with a biofilm an effective amount of two or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In a further aspect, the methods for treating a biofilm in a subject, comprise, or alternatively consist essentially of, or yet further consist of administering to the subject infected with a biofilm an effective amount of three or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In yet a further aspect, the methods for treating a biofilm in a subject, comprise, or alternatively consist essentially of, or yet further consist of administering to the subject infected with a biofilm an effective amount of four or more, or alternatively five or more, or alternatively six or more, or alternatively seven or more, or alternatively eight or more, or alternatively nine or more, or alternatively ten or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme.

This disclosure also relates to methods for preventing the formation of a biofilm in a subject susceptible to developing a biofilm, comprising, or alternatively consisting essentially of, or yet further consisting of administering to the subject an effective amount of an agent that interferes with the binding of a polyamine to the DNA in the biofilm, that in one aspect, wherein the agent is not an HMGB1 protein, fragment or an equivalent of each thereof, and in another aspect, the agent is administered in the absence of a DNAse. In a further aspect, DNAse is administered subsequent to administration of the agent. In one particulate aspect, the DNAse administered is Pulmozyme. In one aspect, the methods for preventing the formation of a biofilm in a subject susceptible to developing a biofilm, comprising, or alternatively consisting essentially of, or yet further consisting of administering to the subject an effective amount of one or more agents that interfere with the binding of a polyamine to the DNA in the biofilm the agent is not an HMGB1 protein, fragment or an equivalent of each thereof, and in another aspect, the agent is administered in the absence of a DNAse. In a further aspect, DNAse is administered subsequent to administration of the agent. In one particulate aspect, the DNAse administered is Pulmozyme. In another aspect, the methods for preventing the formation of a biofilm in a subject susceptible to developing a biofilm, comprising, or alternatively consisting essentially of, or yet further consisting of administering to the subject an effective amount of two or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In a further aspect, the methods for preventing the formation of a biofilm in a subject susceptible to developing a biofilm, comprising, or alternatively consisting essentially of, or yet further consisting of administering to the subject an effective amount of three or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In yet a further aspect, the methods for preventing the formation of a biofilm in a subject susceptible to developing a biofilm, comprising, or alternatively consisting essentially of, or yet further consisting of administering to the subject an effective amount of four or more, or alternatively five or more, or alternatively six or more, or alternatively seven or more, or alternatively eight or more, or alternatively nine or more, or alternatively ten or more agents that interfere with the binding of a polyamine to the DNA in the biofilm, that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme.

This disclosure further relates to methods for treating an infection caused by a bacterium that produces a biofilm in a subject in need thereof, the method comprising, or alternatively consisting essentially of, or yet further consisting of administering to the subject an effective amount of an agent that interferes with the binding of a polyamine to the DNA in the biofilm and an agent that inhibits the replication of the organism, that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In one aspect, the methods for treating an infection caused by a bacterium that produces a biofilm in a subject in need thereof, the method comprising, or alternatively consisting essentially of, or yet further consisting of administering to the subject an effective amount of one or more agents that interfere with the binding of a polyamine to the DNA in the biofilm. In another aspect, the methods for treating an infection caused by a bacterium that produces a biofilm in a subject in need thereof, the method comprising, or alternatively consisting essentially of, or yet further consisting of administering to the subject an effective amount of two or more agents that interfere with the binding of a polyamine to the DNA in the biofilm and an agent that inhibits the replication of the organism. In one aspect, the agents are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In a further aspect, methods for treating an infection caused by a bacterium that produces a biofilm in a subject in need thereof, the method comprising, or alternatively consisting essentially of, or yet further consisting of administering to the subject an effective amount of three or more agents that interfere with the binding of a polyamine to the DNA in the biofilm and an agent that inhibits the replication of the organism that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In yet a further aspect, methods for treating an infection caused by a bacterium that produces a biofilm in a subject in need thereof, the method comprising, or alternatively consisting essentially of, or yet further consisting of administering to the subject an effective amount of four or more, or alternatively five or more, or alternatively six or more, or alternatively seven or more, or alternatively eight or more, or alternatively nine or more, or alternatively ten or more agents that interfere with the binding of a polyamine to the DNA in the biofilm and an agent that inhibits the replication of the organism that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme.

For any of the methods described above, the polyamine can be selected from the group of: putrescine, spermine, cadaverine, 1,3-diaminopropane or spermidine. In one embodiment, for the methods described above, the agent that interferes with the binding of a polyamine to DNA in the biofilm is a tRNA. In another embodiment, the agent is an inhibitor of polyamine synthesis or an agent that inhibits the binding of the polyamine to the DNA. In a second embodiment, the agent comprises, or alternatively consists essentially of, or yet further consisting of a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof. In one aspect, the derivatives of these compounds maintain the same mass to charge ratio. In a third embodiment, the agent comprises, or alternatively consists essentially of, or yet further consisting of an agent that depletes cations from the biofilm, optionally a cation exchange resin, an aminopolycarboxylic acid, a crown ether, an azacrown, or a cryptand. In a fourth embodiment, the agent that depletes cations from the biofilm are selected from the group of: sulfonate, sulfopropyl, phosphocellulose, P11 phosphocellulose, heparin sulfate, or a derivative or analog thereof. In one aspect, a derivative or analog of the agent that depletes cations from the biofilms is a resin that has a net negative charge. In a fifth embodiment, the agent that interferes with the conversion of B-DNA to Z-DNA in the biofilm or its local environment. In a sixth embodiment, the agent comprises, or alternatively consists essentially of, or yet further consists of an anti-B-DNA antibody or fragment or derivative thereof. In one aspect, the polyclonal or monoclonal anti-B-DNA antibody or fragment or derivative thereof recognize B-form DNA over Z form DNA by at least 10-fold in affinity/avidity. In a seventh embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In an eighth embodiment, the agent comprises, or alternatively consists essentially of, or yet further consists of chloroquine or a derivative thereof. In one aspect, the derivatives of the compounds retain the capacity to intercalate between DNA bases. In one aspect, the agent is not an HGMB1 protein or a fragment thereof. In one aspect, the agent that depletes cations from the biofilm has a net negative charge. In another aspect, the agent that depletes cations from the biofilm has a net neutral charge.

Also provided herein are methods for treating a biofilm in a patient suffering from systemic lupus erythematosus (SLE) and/or cystic fibrosis (CF), comprising, or alternatively consisting essentially of, or yet further consisting of administering an effective amount of an agent that interferes with the conversion of B-DNA to Z-DNA in the biofilm or its local environment that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. Methods for treating a biofilm in a patient suffering from systemic lupus erythematosus (SLE) and/or cystic fibrosis (CF) and/or TB, comprising, or alternatively consisting essentially of, or yet further consisting of administering an effective amount of one or more agents that interfere with the conversion of B-DNA to Z-DNA in the biofilm or its local environment are disclosed herein that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In one aspect, the methods for treating a biofilm in a patient suffering from systemic lupus erythematosus (SLE) and/or cystic fibrosis (CF) and/or TB, comprising, or alternatively consisting essentially of, or yet further consisting of administering an effective amount of two or more agents that interfere with the conversion of B-DNA to Z-DNA in the biofilm or its local environment are disclosed herein that in one aspect, are administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In another aspect, the methods for treating a biofilm in a patient suffering from systemic lupus erythematosus (SLE) and/or cystic fibrosis (CF) and/or TB, comprising, or alternatively consisting essentially of, or yet further consisting of administering an effective amount of three or more agents that interfere with the conversion of B-DNA to Z-DNA in the biofilm or its local environment are disclosed herein. In a further aspect, the methods for treating a biofilm in a patient suffering from systemic lupus erythematosus (SLE) and/or cystic fibrosis (CF) and/or TB, comprising, or alternatively consisting essentially of, or yet further consisting of administering an effective amount of four or more, or alternatively five or more, or alternatively six or more, or alternatively seven or more, or alternatively eight or more, or alternatively nine or more, or alternatively ten or more agents that interfere with the conversion of B-DNA to Z-DNA in the biofilm or its local environment are disclosed herein that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In one embodiment, the agent that interferes with the conversion of B-DNA to Z-DNA in the biofilm or its local environment. In a second embodiment, the agent comprises, or alternatively consists essentially of, or yet further consists of an anti-B-DNA antibody or fragment or derivative thereof. In one aspect, the polyclonal or monoclonal anti-B-DNA antibody or fragment or derivative thereof recognize B-form DNA over Z form DNA by at least 10-fold in affinity/avidity. In a third embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In a fourth embodiment, the agent comprises, or alternatively consists essentially of, or yet further consists of chloroquine or a derivative thereof. In one aspect, the derivatives of the compounds retain the capacity to intercalate between DNA bases. The agent is not an HGMB1 protein or a fragment thereof.

Methods for treating a biofilm in a patient suffering from systemic lupus erythematosus (SLE) and/or cystic fibrosis (CF) and/or TB, comprising, or alternatively consisting essentially of, or yet further consisting of administering an effective amount of HMGB1 protein or biologically active fragment thereof and anti-B-DNA antibody or fragment or derivative thereof are also provided herein that in one aspect, is administered in the absence of a DNAse. In a further aspect, DNAse is administered subsequent to administration of the agent. In one particulate aspect, the DNAse administered is Pulmozyme. In one aspect, the polyclonal or monoclonal anti-B-DNA antibody or fragment or derivative thereof recognize B-form DNA over Z form DNA by at least 10-fold in affinity/avidity. The biologically active fragment of HMGB1 may comprise, or alternatively consist essentially of, or yet further consist of one or more of: an A box, a B box, and/or an AB box, a C-terminal fragment or an N-terminal fragment. In a specific embodiment, the biologically active fragment of HMGB1 may comprise, or alternatively consist essentially of, or yet further consist of the B Box domain that is capable of binding DNA. In one aspect, the method for treating a biofilm in a patient suffering from systemic lupus erythematosus (SLE) and/or cystic fibrosis (CF) and/or TB, comprises, or alternatively consists essentially of, or yet further consists of administering an effective amount of chloroquine and anti-B-DNA antibody or fragment or derivative thereof that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. This disclosure also relates to methods for treating a biofilm producing infection incident to administration of a platinum-based chemotherapy in a patient receiving or having received the chemotherapy comprising, or alternatively consisting essentially of, or yet further consisting of administering an effective amount of an agent that interferes with the conversion of B-DNA to Z-DNA in the biofilm or its local environment that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In one aspect, the method comprises, or alternatively consists essentially of, or yet further consists of administering an effective amount of one or more agents that interfere with the conversion of B-DNA to Z-DNA in the biofilm or its local environment that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In a further aspect, the agent comprises, or alternatively consists essentially of, or yet further consists of chloroquine or a derivative thereof. In yet a further aspect, the agent comprises, or alternatively consists essentially of, or yet further consists of an anti-B-DNA antibody or fragment or derivative thereof. In one aspect, the polyclonal or monoclonal anti-B-DNA antibody or fragment or derivative thereof recognize B-form DNA over Z form DNA by at least 10-fold in affinity/avidity. In one embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In one particular aspect, the derivatives of the compounds retain the capacity to intercalate between DNA bases.

This disclosure further relates to methods for treating a biofilm producing infection incident to administration of a platinum-based chemotherapy in a patient receiving or having received the chemotherapy comprising, or alternatively consisting essentially of, or yet further consisting of administering an effective amount of HMGB1 protein or biologically active fragment thereof and anti-B-DNA antibody or fragment or derivative thereof that in one aspect, is administered in the absence of a DNAse. In a further aspect, DNAse is administered subsequent to administration of the agent. In one particulate aspect, the DNAse administered is Pulmozyme. In one aspect, the polyclonal or monoclonal anti-B-DNA antibody or fragment or derivative thereof recognize B-form DNA over Z form DNA by at least 10-fold in affinity/avidity. The biologically active fragment of HMGB1 may comprise, or alternatively consist essentially of, or yet further consist of one or more of: an A box, a B box, and/or an AB box, a C-terminal fragment or an N-terminal fragment. In a specific embodiment, the biologically active fragment of HMGB1 may comprise, or alternatively consist essentially of, or yet further consist of the B Box domain that is capable of binding DNA. Methods for treating a biofilm producing infection incident to administration of a platinum-based chemotherapy in a patient receiving or having received the chemotherapy comprising, or alternatively consisting essentially of, or yet further consisting of administering an effective amount of chloroquine and anti-B-DNA antibody or fragment or derivative thereof are also provided herein that in one aspect, is administered in the absence of a DNAse. In a further aspect, DNAse is administered subsequent to administration of the agent. In one particulate aspect, the DNAse administered is Pulmozyme. In one aspect, the polyclonal or monoclonal anti-B-DNA antibody or fragment or derivative thereof recognize B-form DNA over Z form DNA by at least 10-fold in affinity/avidity.

The methods described above may further comprise, or alternatively consist essentially of, or yet further consist of administering to the subject an effective amount of an agent that interferes with the binding of the eDNA to a DNA binding protein and/or an antibacterial agent that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In one aspect, the methods further comprise, or alternatively consist essentially of, or yet further consist of administering to the subject an effective amount of an agent that interferes with the binding of the eDNA to a DNA binding protein and/or an antibacterial agent that in one aspect, is administered in the absence of a DNAse. In another aspect, the agent is not a HMGB1 protein, fragment or an equivalent of each thereof. In a further aspect, DNAse is administered subsequent to administration of the agent. In one particulate aspect, the DNAse administered is Pulmozyme. In another aspect, the agent that interferes with the binding of the eDNA to the DNA binding protein comprises, or alternatively consists essentially of, or yet further consists of one or more of an anti-DNABII antibody, an anti-IHF antibody and/or an anti-HU antibody, or fragments of each thereof. In one embodiment, the agent that interferes with the binding of the eDNA to a DNA binding protein has a net negative charge. In a second embodiment, the agent that interferes with the binding of the eDNA to a DNA binding protein has a net neutral charge. In a third embodiment, the agent that interferes with the binding of the eDNA to a DNA binding protein has a net positive charge.

Described herein are methods for inhibiting the stability of a biofilm, comprising, or alternatively consisting essentially of, or yet further consisting of contacting the biofilm with an effective amount of an agent that interferes with the binding of a polyamine to DNA in the biofilm that in one aspect, is contacted in the absence of a DNAse. In another aspect, the agent is not a HMGB1 protein, fragment or an equivalent of each thereof. In a further aspect, DNAse is contacted subsequent to contacting with the agent. In one particulate aspect, the DNAse is Pulmozyme. In one aspect, the methods for inhibiting the stability of a biofilm, comprise, or alternatively consist essentially of, or yet further consist of a contacting the biofilm with an effective amount of one or more agents that interfere with the binding of a polyamine to the DNA in the biofilm that in one aspect, are contacted in the absence of a DNAse. In another aspect, the agent is not a HMGB1 protein, fragment or an equivalent of each thereof. In a further aspect, DNAse is contacted subsequent to contacting with the agent. In one particulate aspect, the DNAse is Pulmozyme. In another aspect, the methods for inhibiting the stability of a biofilm, comprise, or alternatively consist essentially of, or yet further consist of a contacting the biofilm with an effective amount of two or more agents that interfere with the binding of a polyamine to the DNA in the biofilm that in one aspect, are contacted in the absence of a DNAse. In another aspect, the agent is not a HMGB1 protein, fragment or an equivalent of each thereof. In a further aspect, DNAse is contacted subsequent to contacting with the agent. In one particulate aspect, the DNAse is Pulmozyme. In a further aspect, the methods for inhibiting the stability of a biofilm, comprise, or alternatively consist essentially of, or yet further consist of a contacting the biofilm with an effective amount of three or more agents that interfere with the binding of a polyamine to the DNA in the biofilm that in one aspect, are contacted in the absence of a DNAse. In another aspect, the agent is not a HMGB1 protein, fragment or an equivalent of each thereof. In a further aspect, DNAse is contacted subsequent to contacting with the agent. In one particulate aspect, the DNAse is Pulmozyme. In a yet further aspect, the methods for inhibiting the stability of a biofilm, comprise, or alternatively consist essentially of, or yet further consist of a contacting the biofilm with an effective amount of four or more, or alternatively five or more, or alternatively six or more, or alternatively seven or more, or alternatively eight or more, or alternatively nine or more, or alternatively ten or more agents that interfere with the binding of a polyamine to the DNA in the biofilm that in one aspect, are contacted in the absence of a DNAse. In another aspect, the agent is not a HMGB1 protein, fragment or an equivalent of each thereof. In a further aspect, DNAse is contacted subsequent to contacting with the agent. In one particulate aspect, the DNAse is Pulmozyme. The contacting may be in vitro or in vivo.

This disclosure also relates to methods for inhibiting the stability of a biofilm, comprising, or alternatively consisting essentially of, or yet further consisting of contacting the biofilm in vitro with an agent that interferes with the binding of a polyamine to the DNA in the biofilm, wherein the contacting comprises, or alternatively consists essentially of, or yet further consists of coating a surface with an effective amount of agent that depletes cations that in one aspect, is contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. In one aspect, the methods for inhibiting the stability of a biofilm, may comprise, or alternatively consist essentially of, or yet further consist of contacting the biofilm in vitro with an effective amount of an agent that interferes with the binding of a polyamine to the DNA in the biofilm, wherein the contacting comprises, or alternatively consists essentially of, or yet further consists of coating a surface with an effective amount of one or more agents that depletes cations that in one aspect, are contacted in the absence of a DNAse, while in another aspect, the DNAse is contacted in accordance with the method. In another aspect, the agent is not a HMGB1 protein, fragment or an equivalent of each thereof In a further aspect, DNAse is contacted subsequent to contacting with the agent. In one particulate aspect, the DNAse is Pulmozyme.

In another aspect, the methods for inhibiting the stability of a biofilm, may comprise, or alternatively consist essentially of, or yet further consist of contacting the biofilm in vitro with an effective amount of an agent that interferes with the binding of a polyamine to the DNA in the biofilm, wherein the contacting comprises, or alternatively consists essentially of, or yet further consists of coating a surface with an effective amount of two or more agents that depletes cations that in one aspect, are contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme.

In a further aspect, the methods for inhibiting the stability of a biofilm, may comprise, or alternatively consist essentially of, or yet further consist of contacting the biofilm in vitro with an effective amount of an agent that interferes with the binding of a polyamine to the DNA in the biofilm, wherein the contacting comprises, or alternatively consists essentially of, or yet further consists of coating a surface with an effective amount of three or more agents that depletes cations that in one aspect, are contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. In yet a further aspect, the methods for inhibiting the stability of a biofilm, may comprise, or alternatively consist essentially of, or yet further consist of contacting the biofilm in vitro with an effective amount of an agent that interferes with the binding of a polyamine to the DNA in the biofilm, wherein the contacting comprises, or alternatively consists essentially of, or yet further consists of coating a surface with an effective amount of four or more, or alternatively five or more, or alternatively six or more, or alternatively seven or more, or alternatively eight or more, or alternatively nine or more, or alternatively ten or more agents that depletes cations. In one embodiment, the agent that interferes with the conversion of B-DNA to Z-DNA in the biofilm or its local environment that in one aspect, are contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. In a second embodiment, the agent comprises, or alternatively consists essentially of, or yet further consists of an anti-B-DNA antibody or fragment or derivative thereof. In one aspect, the polyclonal or monoclonal anti-B-DNA antibody or fragment or derivative thereof recognize B-form DNA over Z form DNA by at least 10-fold in affinity/avidity. In a third embodiment, the agent comprises, or alternatively consists essentially of, or yet further consists of riboflavin, ethidium bromide, bis(methidium)spermine, daunorubicin, TMPyP4, a quaternary benzo[c]phenanthridine alkaloid, quinacrine, 9-amino acridine, or a derivative thereof. In a fifth embodiment, the agent comprises, or alternatively consists essentially of, or yet further consists of chloroquine or a derivative thereof. In one particular aspect, the derivatives of the compounds retain the capacity to intercalate between DNA bases. The agent is not an HGMB1 protein or a fragment thereof.

Further described herein are methods for inhibiting the stability of a biofilm, comprising, or alternatively consisting essentially of, or yet further consisting of contacting the biofilm in vitro with an effective amount of HMGB1 protein or biologically active fragment thereof and anti-B-DNA antibody or fragment or derivative thereof, wherein the contacting comprises, or alternatively consists essentially of, or yet further consists of coating a surface with an effective amount of HMGB1 protein or biologically active fragment thereof and anti-B-DNA antibody or fragment or derivative thereof that in one aspect, is contacted in the absence of a DNAse, while in another aspect, the DNAse is contacted in accordance with the method. In a further aspect, DNAse is contacted subsequent to contacting with the agent. In one particulate aspect, the DNAse is Pulmozyme.

In one aspect, the polyclonal or monoclonal anti-B-DNA antibody or fragment or derivative thereof recognize B-form DNA over Z form DNA by at least 10-fold in affinity/avidity. The biologically active fragment of HMGB1 may comprise, or alternatively consist essentially of, or yet further consist of one or more of: an A box, an AB box, a B box, C-terminal fragment, and/or an N-terminal fragment. In a specific embodiment, the biologically active fragment of HMGB1 may comprise, or alternatively consist essentially of, or yet further consist of the B Box domain that is capable of binding DNA. This disclosure also relates to methods for inhibiting the stability of a biofilm, comprising, or alternatively consisting essentially of, or yet further consisting of contacting the biofilm in vitro with an effective amount of chloroquine and anti-B-DNA antibody or fragment or derivative thereof, wherein the contacting comprises, or alternatively consists essentially of, or yet further consists of coating a surface with an effective amount of chloroquine and anti-B-DNA antibody or fragment or derivative thereof that in one aspect, is contacted in the absence of a DNAse. In a further aspect, DNAse is contacted subsequent to contacting with the agent. In one particulate aspect, the DNAse is Pulmozyme. In one aspect, the polyclonal or monoclonal anti-B-DNA antibody or fragment or derivative thereof recognize B-form DNA over Z form DNA by at least 10-fold in affinity/avidity.

The methods described above may further comprise, or alternatively consist essentially of, or yet further consist of contacting the biofilm with an effective amount of an agent that interferes with the binding of the eDNA to a DNA binding protein and/or an antibacterial agent that in one aspect, is contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. In one aspect, the agent that interferes with the binding of the eDNA to the DNA binding protein comprises, or alternatively consists essentially of, or yet further consists of one or more of an anti-DNABII antibody, an anti-IHF antibody and/or an anti-HU antibody, or fragments of each thereof that in one aspect, is contacted in the absence of a DNAse. In a further aspect, DNAse is contacted subsequent to contacting with the agent. In one particulate aspect, the DNAse is Pulmozyme. In another aspect, the agent is not a HMGB1 protein, fragment or an equivalent of each thereof. In one embodiment, the agent that interferes with the binding of the eDNA to a DNA binding protein has a net negative charge that in one aspect, is contacted in the absence of a DNAse. In a further aspect, DNAse is contacted subsequent to contacting with the agent. In one particulate aspect, the DNAse is Pulmozyme. In a second embodiment, the agent that interferes with the binding of the eDNA to a DNA binding protein has a net neutral charge that in one aspect, is contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. In a third embodiment, the agent that interferes with the binding of the eDNA to a DNA binding protein has a net positive charge. In a further aspect, DNAse is contacted subsequent to contacting with the agent. In one particulate aspect, the DNAse is Pulmozyme.

Provided herein are methods for inhibiting the stability of a biofilm, comprising contacting the biofilm with an agent that interferes with the binding of a polyamine to the DNA in the biofilm that in one aspect, is contacted in the absence of a DNAse, while in another aspect, the DNAse is contacted in accordance with the method. In another aspect, the agent is not a HMGB1 protein, fragment or an equivalent of each thereof. In a further aspect, DNAse is contacted subsequent to contacting with the agent. In one particulate aspect, the DNAse is Pulmozyme. The contacting can be in vitro or in vivo.

Also provided are methods for treating a biofilm in a subject, comprising administering to the subject infected with a biofilm an effective amount of an agent that interferes with the binding of a polyamine to the DNA in the biofilm that in one aspect, is administered in the absence of a DNAse, while in another aspect, the DNAse is administered. In another aspect, the agent is not a HMGB1 protein, fragment or an equivalent of each thereof. In a further aspect, DNAse is administered subsequent to administering with the agent. In one particulate aspect, the DNAse is Pulmozyme.

Further provided are methods for preventing the formation of a biofilm in a subject susceptible to developing a biofilm, comprising administering to the subject an effective amount of an agent that interferes with the binding of a polyamine to the DNA in the biofilm that in one aspect, is administered in the absence of a DNAse while in another aspect, the DNAse is administered. In another aspect, the agent is not a HMGB1 protein, fragment or an equivalent of each thereof. In a further aspect, DNAse is administered subsequent to administering with the agent. In one particulate aspect, the DNAse is Pulmozyme.

Yet further provided are methods for treating an infection caused by an bacteria that produces a biofilm in a subject in need thereof, the method comprising administering to the subject an effective amount of an agent that interferes with the binding of a polyamine to the DNA in the biofilm and an agent that inhibits the replication of the organism that in one aspect, is administered in the absence of a DNAse while in another aspect, the DNAse is administered. In another aspect, the agent is not a HMGB1 protein, fragment or an equivalent of each thereof. In a further aspect, DNAse is administered subsequent to administering with the agent. In one particulate aspect, the DNAse is Pulmozyme.

In one aspect, the wherein the agent is an inhibitor of polyamine synthesis or an agent that inhibits the binding of the polyamine to the DNA. In another aspect, the agent is not a HMGB1 protein, fragment or an equivalent of each thereof. Non-limiting examples of polyamine include: putrescine, spermine, cadaverine, 1,3-diaminopropane or spermidine. In another aspect, the agent comprises a polyamine analog, difluoromethylornithine, trans-4-methylcyclohexylamine, sardomozide (Boc Sciences), methylglyoxal-bis[guanylhydrazone] (methyl-GAG), 1-aminooxy-3-aminopropane (AKos Consulting & Solutions), oxaliplatin, cisplatin, dicyclohexylamine, a derivative of any thereof, or a salt thereof (all of the agents of this paragraph are commercially available from Millipore Sigma unless otherwise indicated). In one aspect, the derivatives of these compounds maintain the same mass to charge ratio.

In a further aspect, the agent comprises an agent that depletes cations from the biofilm, optionally a cation exchange resin, an aminopolycarboxylic acid, a crown ether, an azacrown, or a cryptand (various representative compounds of each class of agent available from Millipore Sigma). In another aspect, the agent is not a HMGB1 protein, fragment or an equivalent of each thereof. Non-limiting examples of a cation exchange resin include sulfonate, sulfopropyl, phosphocellulose, P11 phosphocellulose, heparin sulfate, or resins containing a derivative or analog thereof. In one embodiment, the agent that depletes cations from the biofilm has a net negative charge. In one embodiment, the agent that depletes cations from the biofilm has a net neutral charge.

In one embodiment, provided herein are methods for inhibiting the stability of a biofilm, comprising contacting the biofilm in vitro with an agent that interferes with the binding of a polyamine to the DNA in the biofilm and contacting comprises coating a surface with the agent that depletes cations that in one aspect, is administered in the absence of a DNAse while in another aspect, the DNAse is administered. In another aspect, the agent is not a HMGB1 protein, fragment or an equivalent of each thereof. In a further aspect, DNAse is administered subsequent to administering with the agent. In one particulate aspect, the DNAse is Pulmozyme.

In one aspect of the above methods, the agent that interferes with the conversion of B-DNA to Z-DNA in the biofilm or its local environment that in one aspect, is administered in the absence of a DNAse while in another aspect, the DNAse is administered. In a further aspect, DNAse is administered subsequent to administering with the agent. In one particulate aspect, the DNAse is Pulmozyme. Examples of such include an anti-B-DNA antibody or fragment or derivative thereof. In a further aspect, the agent comprises an agent from the group of: riboflavin, ethidium bromide, bis(methidium)spermine, (Dervan et al. (1978) 100(6):1968-1970) daunorubicin, TMPyP4, a quaternary benzo[c]phenanthridine alkaloid, (Rajecky et al. (2015); Le et al. (2004) 69(8):2768-2772) quinacrine, 9-amino acridine, or a derivative thereof (all of the agents of this further aspect are commercially available from Millipore Sigma unless otherwise indicated). The agent is not an HGMB1 protein or a fragment thereof.

Further provided are methods for treating a biofilm in a patient suffering from systemic lupus erythematosus (SLE) and/or cystic fibrosis (CF), comprising administering an effective amount of an agent that interferes with the conversion of B-DNA to Z-DNA in the biofilm or its local environment that in one aspect, is administered in the absence of a DNAse while in another aspect, the DNAse is administered. In a further aspect, DNAse is administered subsequent to administering with the agent. In one particulate aspect, the DNAse is Pulmozyme. Examples of such include anti-B-DNA antibody or fragment or derivative thereof. In a further aspect, the agent comprises an agent from the group of: riboflavin, ethidium bromide, bis(methidium)spermine, daunorubicin, TMPyP4, a quaternary benzo[c]phenanthridine alkaloid, quinacrine, 9-amino acridine, or a derivative thereof. In one aspect, the method is performed in the absence of a DNAse, and in one aspect treatment of CF is performed in the absence of a DNAse. The agent is not an HGMB1 protein or a fragment thereof.

In another aspect, provided herein are methods for treating a biofilm producing infection incident to administration of a platinum-based chemotherapy in a patient receiving or having received the chemotherapy, the method comprising administering an effective amount of an agent that interferes with the conversion of B-DNA to Z-DNA in the biofilm or its local environment that in one aspect, is administered in the absence of a DNAse while in another aspect, the DNAse is administered. In a further aspect, DNAse is administered subsequent to administering with the agent. In one particulate aspect, the DNAse is Pulmozyme. Examples of such include an anti-B-DNA antibody or fragment or derivative thereof. In a further aspect, the gent comprises an agent from the group of: riboflavin, ethidium bromide, bis(methidium)spermine, daunorubicin, TMPyP4, a quaternary benzo[c]phenanthridine alkaloid, quinacrine, 9-amino acridine, or a derivative thereof. The agent is not an HGMB1 protein or a fragment thereof.

The above noted methods can further comprise contacting the biofilm (when in vitro) or administering to the subject an effective amount of an agent that interferes with the binding of the eDNA to a DNA binding protein and/or an antibacterial agent that in one aspect, is administered in the absence of a DNAse while in another aspect, the DNAse is administered. In another aspect, the agent is not a HMGB1 protein, fragment or an equivalent of each thereof. In a further aspect, DNAse is administered subsequent to administering with the agent. In one particulate aspect, the DNAse is Pulmozyme. In an embodiment, the agent that interferes with the binding of the eDNA to a DNA binding protein has a net positive charge. In an embodiment, the agent that interferes with the binding of the eDNA to a DNA binding protein has a net negative charge. In an embodiment, the agent that interferes with the binding of the eDNA to a DNA binding protein has a net neutral charge.

When practiced in vitro, the methods are useful to screen for or confirm agents having the same, similar or opposite ability as the polypeptides, polynucleotides, antibodies, host cells, small molecules and compositions disclosed herein. Alternatively, they can be used to identify which agent is best suited to treat a microbial infection or if the treatment has been effective. For example, one can screen for new agents or combination therapies by having two samples containing for example, the DNABII polypeptide and microbial DNA and the agent to be tested. The second sample contains the DNABII polypeptide and microbial DNA and an agent known to active, e.g., an anti-IHF antibody or a small molecule to serve as a positive control. In a further aspect, several samples are provided and the agents are added to the system in increasing dilutions to determine the optimal dose that would likely be effective in treating a subject in the clinical setting. As is apparent to those of skill in the art, a negative control containing the DNABII polypeptide and the microbial DNA can be provided. In a further aspect, the DNABII polypeptide and the microbial DNA are detectably labeled, for example with luminescent molecules that will emit a signal when brought into close contact with each other. The samples are contained under similar conditions for an effective amount of time for the agent to inhibit, compete or titrate the interaction between the DNABII polypeptide and microbial DNA and then the sample is assayed for emission of signal from the luminescent molecules. If the sample emits a signal, then the agent is not effective to inhibit binding.

In another aspect, the in vitro method is practiced in a miniaturized chamber slide system wherein the microbial (such as a bacterial) isolate causing an infection could be isolated from the human/animal then cultured to allow it to grow as a biofilm in vitro. The agent (such as anti-DNABII or IHF antibody) or a test or potential agent is added alone or in combination with another agent to the culture with or without increasing dilutions of the potential agent or agent such as an anti-DNABII or IHF (or other antibody, small molecule, agent, etc.) to find the optimal dose that would likely be effective at treating that patient when delivered to the subject where the infection existed. As apparent to those of skill in the art, a positive and negative control can be performed simultaneously.

In a further aspect, the method is practiced in a high throughput platform with the agent (such as anti-DNABII or IHF antibody) and/or potential agent (alone or in combination with another agent) in a flow cell. The agent (such as anti-DNABII or IHF antibody) or potential agent biofilm is added alone or in combination with another agent to the culture with or without increasing dilutions of the potential agent or agent such as an anti-DNABII or IHF (or other antibody, small molecule, agent, etc.) to find the optimal dose that would likely be effective at treating that patient when delivered to the subject where the infection existed. Biofilm isolates are sonicated to separate biofilm bacteria from DNABII polypeptide such as IHF bound to microbial DNA. The DNABII polypeptide-DNA complexes are isolated by virtue of the anti-DNABII or IHF antibody on the platform. The microbial DNA is then released with e.g., a salt wash, and used to identify the biofilm bacteria added. The freed DNA is then identified, e.g., by PCR sequenced. If DNA is not freed, then the agent(s) successfully performed or bound the microbial DNA. If DNA is found in the sample, then the agent did not interfere with DNABII polypeptide-microbial DNA binding. As is apparent to those of skill in the art, a positive and/or negative control can be simultaneously performed.

The above methods also can be used as a diagnostic test since it is possible that a given bacterial species will respond better to reversal of its biofilm by one agent more than another, this rapid high throughput assay system could allow one skilled the art to assay a panel of possible anti-DNABII or IHF-like agents to identify the most efficacious of the group.

The advantage of these methods is that most clinical microbiology labs in hospitals are already equipped to perform these sorts of assays (i.e., determination of MIC, MBC values) using bacteria that are growing in liquid culture (or planktonically). As is apparent to those of skill in the art, bacteria generally do not grow planktonic ally when they are causing diseases. Instead they are growing as a stable biofilm and these biofilms are significantly more resistant to treatment by antibiotics, antibodies or other therapeutics. This resistance is why most MIC/MBC values fail to accurately predict efficacy in vivo. Thus, by determining what “dose” of agent could reverse a bacterial biofilm in vitro (as described above) Applicants' pre-clinical assay would be a more reliable predictor of clinical efficacy, even as an application of personalized medicine.

In addition to the clinical setting, the methods can be used to identify the microbe causing the infection and/or confirm effective agents in an industrial setting. Thus, the agents can be used to treat, inhibit or titrate a biofilm in an industrial setting.

In a further aspect of the above methods, an antibiotic or antimicrobial known to inhibit growth of the underlying infection is added sequentially or concurrently, to determine if the infection can be inhibited. It is also possible to add the agent to the microbial DNA or DNABII polypeptide before adding the missing complex to assay for biofilm inhibition.

When practiced in vivo in non-human animal such as a chinchilla, the method provides a pre-clinical screen to identify agents that can be used alone or in combination with other agents to break down biofilms.

In another aspect, provided herein is a method of inhibiting, preventing or breaking down a biofilm in a subject by administering to the subject an effective amount of an agent, thereby inhibiting, preventing or breaking down the microbial biofilm. Non-limiting examples of such subjects include mammals, e.g., pets, and human patients.

The agents and compositions disclosed herein can be concurrently or sequentially administered with other antimicrobial agents and/or surface antigens. In one particular aspect, administration is locally to the site of the infection by direct injection or by inhalation for example. Other non-limiting examples of administration include by one or more method comprising transdermally, urethrally, sublingually, rectally, vaginally, ocularly, subcutaneous, intramuscularly, intraperitoneally, intranasally, by inhalation or orally.

Microbial infections and disease that can be treated by the methods disclosed herein include infection by the organisms Streptococcus agalactiae, Neisseria meningitidis, Treponemes, denticola, pallidum, Burkholderia cepacia, or Burkholderia pseudomallei. In one aspect, the microbial infection is one or more of Haemophilus influenzae (nontypeable), Moraxella catarrhalis, Streptococcus pneumoniae, Streptococcus pyogenes, Pseudomonas aeruginosa, Mycobacterium tuberculosis. These microbial infections may be present in the upper, mid and lower airway (otitis, sinusitis, bronchitis but also exacerbations of chronic obstructive pulmonary disease (COPD), chronic cough, complications of and/or primary cause of cystic fibrosis (CF) and community acquired pneumonia (CAP). Thus, by practicing the in vivo methods disclosed herein, these diseases and complications from these infections can also be prevented or treated.

Infections might also occur in the oral cavity (caries, periodontitis) and caused by Streptococcus mutans, Porphyromonas gingivalis, Aggregatibacter actinomvctemcomitans. Infections might also be localized to the skin (abscesses, ‘staph’ infections, impetigo, secondary infection of burns, Lyme disease) and caused by Staphylococcus aureus, Staphylococcus epidermidis, Pseudomonas aeruginosa and Borrelia burdorferi. Infections of the urinary tract (UTI) can also be treated and are typically caused by Escherichia coli. Infections of the gastrointestinal tract (GI) (diarrhea, cholera, gall stones, gastric ulcers) are typically caused by Salmonella enterica serovar, Vibrio cholerae and Helicobacter pylori. Infections of the genital tract include and are typically caused by Neisseria gonorrhoeae. Infections can be of the bladder or of an indwelling device caused by Enterococcus faecalis. Infections associated with implanted prosthetic devices, such as artificial hip or knee replacements, or dental implants, or medical devices such as pumps, catheters, stents, or monitoring systems, typically caused by a variety of bacteria, can be treated by the methods disclosed herein. These devices can be coated or conjugated to an agent as described herein. Thus, by practicing the in vivo methods disclosed herein, these diseases and complications from these infections can also be prevented or treated.

Infections caused by Streptococcus agalactiae can also be treated by the methods disclosed herein and it is the major cause of bacterial septicemia in newborns. Infections caused by Neisseria meningitidis which can cause meningitis can also be treated.

Thus, routes of administration applicable to the methods disclosed herein include intranasal, intramuscular, urethrally, intratracheal, subcutaneous, intradermal, transdermal, topical application, intravenous, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery include systemic or localized routes. In general, routes of administration suitable for the methods disclosed herein include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The agents disclosed herein can also be delivered to the subject by enteral administration. Enteral routes of administration include, but are not limited to, oral and rectal (e.g., using a suppository) delivery.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods disclosed herein, the agent is administered by inhalation, injection or orally on a continuous, daily basis, at least once per day (QD), and in various embodiments two (BID), three (TID), or even four times a day. Typically, the therapeutically effective daily dose can be at least about 1 mg, or at least about 10 mg, or at least about 100 mg, or about 200 to about 500 mg, and sometimes, depending on the compound, up to as much as about 1 g to about 2.5 g.

Dosing of can be accomplished in accordance with the methods disclosed herein using capsules, tablets, oral suspension, suspension for intra-muscular injection, suspension for intravenous infusion, get or cream for topical application, or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In certain embodiments, compositions exhibit high therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies (in certain embodiments, within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Antibodies and Derivatives Thereof

This disclosure also provides an antibody that binds and/or specifically recognizes and binds a B DNA for use in the methods disclosed herein. The antibody can be any of the various antibodies described herein, non-limiting, examples of such include a polyclonal antibody, a monoclonal antibody, a chimeric antibody, a human antibody, a veneered antibody, a diabody, a humanized antibody, an antibody derivative, a recombinant humanized antibody, or a derivative or fragment of each thereof. In one aspect, the fragment comprises, or alternatively consists essentially of, or yet further consists of the CDR of the antibody. In one aspect, the antibody is detectably labeled or further comprises a detectable label conjugated to it. Also provided is a hybridoma cell line that produces a monoclonal antibody disclosed herein. Compositions comprising or alternatively consisting essentially of or yet further, consisting of one or more of the above embodiments are further provided herein. Further provided are polynucleotides that encode the amino acid sequence of the antibodies and fragments as well as methods to produce recombinantly or chemically synthesize the antibody polypeptides and fragments thereof. The antibody polypeptides can be produced in a eukaryotic or prokaryotic cell, or by other methods known in the art and described herein.

Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a stow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods known in the art some of which are described in U.S. Pat. Nos. 7,279,559; 7,119,179; 7,060,800; 6,709,659; 6,656,746; 6,322,788; 5,686,073; and 5,670,153.

Monoclonal antibodies can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, P3X63Ag8,653, Sp2 SA3, Sp2 MAI, Sp2 SS1, Sp2 SA5, U397, MIA 144, ACT IV, MOLT4, DA-1, JURKAT, WEHI, K-562, COS, RAJI, NIH 313, HL-60, MLA 144, NAMAIWA, NEURO 2A, CHO, PerC.6, YB2/O) or the like, or heteromyelomas, fusion products thereof, or any cell or fusion cell derived there from, or any other suitable cell line as known in the art (see, those at the following web addresses, e.g., atcc.org, lifetech.com, last accessed on Nov. 26, 2007), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, in particular embodiments, the spleen or lymph nodes, of humans or other suitable animals that have been immunized with the antigen of interest and then screened for the activity of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, cDNA, or the like, display library; e.g., as available from various commercial vendors such as MorphoSys (Martinsreid/Planegg, Del.), Biolnvent (Lund, Sweden), Affitech (Oslo, Norway) using methods known in the art. Art known methods are described in the patent literature some of which include U.S. Pat. Nos. 4,704,692; 5,723,323; 5,763,192; 5,814,476; 5,817,483; 5,824,514; and 5,976,862. Alternative methods rely upon immunization of transgenic animals (e.g., SCID mice, Nguyen et al. (1977) Microbiol. Immunol. 41:901-907 (1997); Sandhu et al. (1996) Crit, Rev. Biotechnol. 16:95-118; Eren et al. (1998) Mumma 93:154-161 that are capable of producing a repertoire of human antibodies, as known in the art and/or as described herein. Such techniques, include, but are not limited to, ribosome display Wanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052; Wen et al. (1987) J. Immunol 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134).

Antibody derivatives of the present disclosure can also be prepared by delivering a polynucleotide encoding an antibody disclosed herein to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fe-mediated cellular toxicity, and glycoproteins so generated.

The antibodies disclosed herein also include derivatives that are modified by the covalent attachment of any type of molecule to the antibody such that covalent attachment does not prevent the antibody from generating an anti-idiotypic response. Antibody derivatives include, but are not limited to, antibodies that have been modified by glycosylation, acetylation, pegylation, phosphorylation, amidation, derivatization by known protecting/blocking groups, proteolytic cleavage, linkage to a cellular ligand or other protein, etc. Additionally, the derivatives may contain one or more non-classical amino acids.

Antibody derivatives also can be prepared by delivering a polynucleotide disclosed herein to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize has been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFvs), including tobacco seeds and potato tubers. See, e.g., Conrad et al. (1998) Plant Mol. Biol. 38:101-109 and references cited therein. Thus, antibodies can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids or variable or constant regions from other isotypes.

In general, the CDR residues are directly and most substantially involved in influencing antigen binding. Humanization or engineering of antibodies can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Chimeric, humanized or primatized antibodies of the present disclosure can be prepared based on the sequence of a reference monoclonal antibody prepared using standard molecular biology techniques. DNA encoding the heavy and light chain immunoglobulins can be obtained from the hybridoma of interest and engineered to contain non-reference (e.g., human) immunoglobulin sequences using standard molecular biology techniques. For example, to create a chimeric antibody, the murine variable regions can be linked to human constant regions using methods known in the art (U.S. Pat. No. 4,816,567). To create a humanized antibody, the murine CDR regions can be inserted into a human framework using methods known in the art (U.S. Pat. Nos. 5,225,539 and 5,530,101; 5,585,089; 5,693,762; and 6,180,370). Similarly, to create a primatized antibody the murine CDR regions can be inserted into a primate framework using methods known in the art (WO 93/02108 and WO 99/55369).

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies disclosed herein also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies disclosed herein can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al., which discloses antibody variants that have one or more amino acids inserted into a hypervariable region of the parent antibody and a binding affinity for a target antigen which is at least about two fold stronger than the binding affinity of the parent antibody for the antigen).

The term “antibody derivative” further includes engineered antibody molecules, fragments and single domains such as scFv, dAbs, nanobodies, minibodies, Unibodies, and Affibodies & Hudson (2005) Nature Biotech 23(9):1126-36; U.S. Pat. Application Publication No. 2006/0211088; PCT International Application Publication No. WO 2007/059782; U.S. Pat. No. 5,831,012).

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Ed segments (V_{H}-C_{H}1-VH-C_{H}1) which form a pair of antigen binding regions. Linear antibodies can be bispecific or monospecific.

The antibodies disclosed herein can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from a prokaryotic host as described above. A number of antibody production systems are described in Birch & Radner (2006) Adv. Drug Delivery Rev. 58: 671-685.

If an antibody being tested binds with protein or polypeptide, then the antibody being tested and the antibodies provided by this disclosure are equivalent. It also is possible to determine without undue experimentation, whether an antibody has the same specificity as the antibody disclosed herein by determining whether the antibody being tested prevents an antibody disclosed herein from binding the protein or polypeptide with which the antibody is normally reactive. If the antibody being tested competes with the antibody disclosed herein as shown by a decrease in binding by the monoclonal antibody disclosed herein, then it is likely that the two antibodies bind to the same or a closely related epitope. Alternatively, one can pre-incubate the antibody disclosed herein with a protein with which it is normally reactive, and determine if the antibody being tested is inhibited in its ability to bind the antigen. If the antibody being tested is inhibited then, in all likelihood, it has the same, or a closely related, epitopic specificity as the antibody disclosed herein.

The term “antibody” also is intended to include antibodies of all immunoglobulin isotypes and subclasses. Particular isotypes of a monoclonal antibody can be prepared either directly by selecting from an initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307. Alternatively, recombinant DNA techniques may be used.

The isolation of other monoclonal antibodies with the specificity of the monoclonal antibodies described herein can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody of interest.

In some aspects disclosed herein, it is useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The variable region of the antibodies of the present disclosure can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²⁵I, ¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydral groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabinc, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americanaproteins (PAPI, PAPII, and PAP-S), Momordica charantia inhibitor, curcin, crotin, Sapaonaria officinalis inhibitor, gelonin, mitogellin, restrietocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (Yu et al. 1994 Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

In some of the aspects of the antibodies provided herein, the antibody is a full-length antibody.

In some of the aspects of the antibodies provided herein, the antibody is a monoclonal antibody.

In some of the aspects of the antibodies provided herein, the antibody is chimeric or humanized.

In some of the aspects of the antibodies provided herein, the antibody is selected from the group consisting of Fab, F(ab)′2, Fab′, scFv, and Fv.

In some of the aspects of the antibodies provided herein, the antibody comprises an Fc domain. In some of the aspects of the antibodies provided herein, the antibody is a non-human animal such as a rat, sheep, bovine, canine, feline or rabbit antibody. In some of the aspects of the antibodies provided herein, the antibody is a human or humanized antibody or is non-immunogenic in a human.

In some of the aspects of the antibodies provided herein, the antibody comprises a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.

1) Amino acids with basic side chains: lysine, arginine, histidine.

2) Amino acids with acidic side chains: aspartic acid, glutamic acid

3) Amino acids with uncharged polar side chains: asparagine, glutamine, serine, threonine, tyrosine.

4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind a DNABII protein with similar specificity and sensitivity profiles as the disclosed antibodies. This may be tested by way of the binding assays.

In a further aspect, the antibodies are characterized by being both immunodominant and immunoprotective, as determined using appropriate assays and screens.

### Functional Analysis with Antibodies

Antibodies disclosed herein can be used to purify the polypeptides disclosed herein and to identify biological equivalent polypeptide and/or polynucleotides. They also can be used to identify agents that modify the function of the polypeptides disclosed herein. These antibodies include polyclonal antisera, monoclonal antibodies, and various reagents derived from these preparations that are familiar to those practiced in the art and described above.

Antibodies that neutralize the activities of proteins encoded by identified genes can also be used in vivo and in vitro to demonstrate function by adding such neutralizing antibodies into in vivo and in vitro test systems. They also are useful as pharmaceutical agents to modulate the activity of polypeptides disclosed herein.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

The antibodies disclosed herein may be used for vaccination or to boost vaccination, alone or in combination with peptides or protein-based vaccines or dendritic-cell based vaccines.

### Compositions

This disclosure further provides composition comprising, or alternatively consisting essentially of, or yet further consisting of one, two or more, three or more of: an agent that interferes with the binding of a polyamine to DNA in the biofilm, an agent that depletes cations from the biofilm, an agent that interferes with the conversion of B-DNA to Z-DNA in the biofilm or its local environment, an agent that interferes with the binding of the eDNA to a DNA binding protein and/or an antibacterial agent. In one aspect, the composition does not comprise, consist essentially of, or yet further consist of a HMB1 protein, fragment or an equivalent thereof. In another aspect it comprises, consists essentially of, or yet further consists of, a DNAse. In a further aspect it does not comprise, consist essentially of, or yet further consist of, a DNAse. In one embodiment, the composition comprises, or alternatively consists essentially of, or yet further consists of an agent that interferes with the binding of a polyamine to DNA in the biofilm and an agent that interferes with the conversion of B-DNA to Z-DNA in the biofilm or its local environment. In a second embodiment, the composition comprises, or alternatively consists essentially of, or yet further consists of an agent that depletes cations from the biofilm, an agent that interferes with the conversion of B-DNA to Z-DNA in the biofilm or its local environment and an agent that interferes with the binding of the eDNA to a DNA binding protein. In a third embodiment, the composition comprises, or alternatively consists essentially of, or yet further consists of an agent that interferes with the conversion of B-DNA to Z-DNA in the biofilm or its local environment and an agent that interferes with the binding of the eDNA to a DNA binding protein. In a fourth embodiment, the composition comprises, or alternatively consists essentially of, or yet further consists of an agent an agent that interferes with the binding of a polyamine to DNA in the biofilm and an agent that interferes with the binding of the eDNA to a DNA binding protein. In a fifth embodiment, the composition comprises, or alternatively consists essentially of, or yet further consists of an agent an agent that interferes with the binding of a polyamine to DNA in the biofilm and an agent that interferes with the binding of the eDNA to a DNA binding protein and/or an antibacterial agent. In a sixth embodiment, the composition comprises, or alternatively consists essentially of, or yet further consists of an agent an agent that interferes with the binding of a polyamine to DNA in the biofilm, an agent that depletes cations from the biofilm and an agent that interferes with the binding of the eDNA to a DNA binding protein. In a seventh embodiment, the composition comprises, or alternatively consists essentially of, or yet further consists of an agent an agent that interferes with the binding of a polyamine to DNA in the biofilm, an agent that depletes cations from the biofilm and an agent that interferes with the conversion of B-DNA to Z-DNA in the biofilm or its local environment.

The compositions of this disclosure may further comprise, or alternatively consist essentially of, or yet further consist of a pharmaceutically acceptable carrier.

In one aspect, the agent that interferes with the binding of a polyamine to DNA in the biofilm comprises one or more of: a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin and/or dicyclohexylamine, a derivative of any thereof, or a salt thereof. In another aspect, the agent that depletes cations from the biofilm comprises one or more of: a cation exchange resin, an aminopolycarboxylic acid, a crown ether, an azacrown, or a cryptand, sulfonate, sulfopropyl, phosphocellulose, P11 phosphocellulose and/or heparin sulfate, or a derivative or analog thereof. In yet another aspect, the agent that interferes with the conversion of B-DNA to Z-DNA in the biofilm or its local environment comprises one or more of: HMGB1 protein, fragment or an equivalent of each thereof, an anti-B-DNA antibody or fragment or derivative thereof, and/or chloroquine, or a derivative of any thereof. In one particular aspect, the agent that interferes with the binding of the eDNA to a DNA binding protein comprises one or more of: an anti-DNABII antibody, an anti-IHF antibody and/or an anti-HU antibody, or fragments of each thereof.

Compositions are further provided. The compositions comprise a carrier and one or more of an isolated polypeptide disclosed herein, an isolated polynucleotide disclosed herein, a vector disclosed herein, an isolated host cell disclosed herein, a small molecule or an antibody disclosed herein. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, a small molecule, an isolated host cell disclosed herein, or an antibody of the disclosure, formulated with one or more pharmaceutically acceptable substances.

For oral preparations, any one or more of an isolated or recombinant polypeptide as described herein, an isolated or recombinant polynucleotide as described herein, a vector as described herein, an isolated host cell as described herein, a small molecule or an antibody as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated polypeptide disclosed herein, an isolated polynucleotide disclosed herein, a vector disclosed herein, an isolated host cell disclosed herein, or an antibody disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. A non-limiting example of such is a antimicrobial agent such as other vaccine components such as surface antigens, e.g., an OMP P5, OMP 26, OMP P2, or Type IV Pilin protein (see Jurcisek and Bakaletz (2007) J. of Bacteriology 189(10):3868-3875 and Murphy, T F, Bakaletz, L O and Smeesters, P R (2009) The Pediatric Infectious Disease Journal, 28:S121-S126) and antibacterial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of an isolated polypeptide disclosed herein, an isolated polynucleotide disclosed herein, a vector disclosed herein, a small molecule for use in the disclosure, an isolated host cell disclosed herein, or an antibody disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of an isolated polypeptide disclosed herein, an isolated polynucleotide disclosed herein, a vector disclosed herein, an isolated host cell disclosed herein, or an antibody disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the agent (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The present disclosure provides methods and compositions for the administration of a one or more of an agent to a host (e.g., a human) for the treatment of a microbial infection. In various embodiments, these methods disclosed herein span almost any available method and route suitable for drug delivery, including in vivo and ex vivo methods, as well as systemic and localized routes of administration.

### Screening Assays

The present disclosure provides methods for screening for equivalent agents, such as equivalent monoclonal antibodies to a polyclonal antibody as described herein and various agents that modulate the activity of the active agents and pharmaceutical compositions disclosed herein or the function of a polypeptide or peptide product encoded by the polynucleotide disclosed herein. For the purposes of this disclosure, an “agent” is intended to include, but not be limited to a biological or chemical compound such as a simple or complex organic or inorganic molecule, a peptide, a protein (e.g., antibody), a polynucleotide anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

As is apparent to one of skill in the art, suitable cells can be cultured in micro-titer plates and several agents can be assayed at the same time by noting genotypic changes, phenotypic changes or a reduction in microbial titer.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined,

When the agent is an antibody or antigen binding fragment, the agent can be contacted or incubated with the target antigen and polyclonal antibody as described herein under conditions to perform a competitive ELISA. Such methods are known to the skilled artisan.

The assays also can be performed in a subject. When the subject is an animal such as a rat, chinchilla, mouse or simian, the method provides a convenient animal model system that can be used prior to clinical testing of an agent in a human patient. In this system, a candidate agent is a potential drug if symptoms of the disease or microbial infection is reduced or eliminated, each as compared to untreated, animal having the same infection. It also can be useful to have a separate negative control group of cells or animals that are healthy and not treated, which provides a basis for comparison.

The agents and compositions can be used in the manufacture of medicaments and for the treatment of humans and other animals by administration in accordance with conventional procedures, such as an active ingredient in pharmaceutical compositions.

### Combination Therapy

The compositions and related methods of the present disclosure may be used in combination with the administration of other therapies. These include, but are not limited to, the administration of DNase enzymes, antibiotics, antimicrobials, or other antibodies. In one aspect, the agent is administered in the absence of a DNase enzyme.

In other embodiments, the methods and compositions can be combined with antibiotics and/or antimicrobials. Antimicrobials are substances that kill or inhibit the growth of microorganisms such as bacteria, fungi, or protozoans. Although biofilms are generally resistant to the actions of antibiotics, compositions and methods described herein can be used to sensitize the infection involving a biofilm to traditional therapeutic methods for treating infections. In other embodiments, the use of antibiotics or antimicrobials in combination with methods and compositions described herein allow for the reduction of the effective amount of the antimicrobial and/or biofilm reducing agent. Some non-limiting examples of antimicrobials and antibiotics useful in combination with methods of the current disclosure include amoxicillin, amoxicillin-clavulanate, cefdinir, azithromycin, and sulfamethoxazole-trimethoprim. The therapeutically effective dose of the antimicrobial and/or antibiotic in combination with the biofilm reducing agent can be readily determined by traditional methods. In some embodiments the dose of the antimicrobial agent in combination with the biofilm reducing agent is the average effective dose which has been shown to be effective in other bacterial infections, for example, bacterial infections wherein the etiology of the infection does not include a biofilm. In other embodiments, the dose is 0.1, 0.15, 0.2, 0.25, 0.30, 0.35, 0.40, 0.45, 0.50, 0.55, 0.60, 0.65, 0.70, 0.75, 0.8, 0.85, 0.9, 0.95, 1.1, 1.2, 1.3, 1.4, 1.5, 1.6, 1.7, 1.8, 1.9, 2.0, 2.5, 3.0 or 5 times the average effective dose. The antibiotic or antimicrobial can be added prior to, concurrent with, or subsequent to the addition of the anti-DNABII antibody.

In other embodiments, the methods and compositions can be combined with antibodies that treat the bacterial infection. One example of an antibody useful in combination with the methods and compositions described herein is an antibody directed against an unrelated outer membrane protein (i.e., OMP P5). Treatment with this antibody alone does not debulk a biofilm in vitro. Combined therapy with this antibody and a biofilm reducing agent results in a greater effect than that which could be achieved by either reagent used alone at the same concentration. Other antibodies that may produce a synergistic effect when combined with a biofilm reducing agent or methods to reduce a biofilm include anti-rsPilA anti-OMP26, anti-OMP P2, and anti-whole OMP preparations.

The compositions and methods described herein can be used to sensitize the bacterial infection involving a biofilm to common therapeutic modalities effective in treating bacterial infections without a biofilm but are otherwise ineffective in treating bacterial infections involving a biofilm. In other embodiments, the compositions and methods described herein can be used in combination with therapeutic modalities that are effective in treating bacterial infections involving a biofilm, but the combination of such additional therapy and biofilm reducing agent or method produces a synergistic effect such that the effective dose of either the biofilm reducing agent or the additional therapeutic agent can be reduced. In other instances, the combination of such additional therapy and biofilm reducing agent or method produces a synergistic effect such that the treatment is enhanced. An enhancement of treatment can be evidenced by a shorter amount of time required to treat the infection.

The additional therapeutic treatment can be added prior to, concurrent with, or subsequent to methods or compositions used to reduce the biofilm, and can be contained within the same formation or as a separate formulation.

### Kits

Provided herein are kits comprising, or alternatively consisting essentially of, or yet further consisting of the composition disclosed herein and instructions for use. In one aspect, the instruction for use provide directions to conduct any of the methods disclosed herein. In one aspect, one or more, two or more or three or more of the agents for use in the disclosed methods are packaged independently or together in the kit.

Kits containing the agents and instructions necessary to perform the in vitro and in vivo methods as described herein also are claimed. Accordingly, the disclosure provides kits for performing these methods which may include as disclosed herein as well as instructions for carrying out the methods disclosed herein such as collecting tissue and/or performing the screen, and/or analyzing the results, and/or administration of an effective amount of an agent as defined herein. These can be used alone or in combination with other suitable antimicrobial agents.

The following examples are intended to illustrate, and not limit the embodiments disclosed herein.

### Experiment No. 1

Polyamines are ubiquitous small aliphatic polycations produced and utilized by nearly all living organisms. Michael et al. (2016) J Biol Chem. 291(29):14896-903; D'Agostino et al. (2005) FEBS J. 272(15):3777-87. Derived from amino acids, they play roles in a multitude of cellular functions central to growth and proliferation, including transcription, translation, transcriptional regulation, autophagy, and stress resistance. Miller-Fleming et al. (2015) J Mol Biol 427(21):3389-406. There are multiple pathways for polyamine synthesis, and their presence in the metabolic repertoire varies among species. Michael et al. (2016) Biochem J. 473(15):2315-29. Due to the nonspecific nature of their electrostatic-mediated interactions, polyamine synthesis is tightly regulated via a combination of transcription, translation, and protein degradation mechanisms. Miller-Fleming et al. (2015) J Mol Biol 427(21):3389-406.

There are five primary polyamine molecules produced by living organisms; spermine, spermidine, putrescine, cadaverine, and 1,3-diaminopropane. Miller-Fleming et al. (2015) J Mol Biol 427(21):3389-406. Additional types of polyamines are produced in a more species-specific manner. Each polyamine has slightly different attributes, owing to differences in length and number of amine groups that dictate cationic character and distribution of charges. Michael et al. (2016) J Biol Chem. 291(29):14896-903. This variance allows for some level of specificity in polyamine activity, as well as directs assembly of polyamine aggregates. D'Agostino et al. (2005) FEBS J. 272(15):3777-87; D'Agostino et al. (2006) IUBMB Life 58(2):75-82.

Polyamine function is concentration dependent as is evidenced by the multiple disease states that are correlated with dysregulation of polyamine concentrations. Miller-Fleming et al. (2015) J Mol Biol 427(21):3389-406. General metabolic processes can be disrupted due to altered levels of polyamines, and specific processes can be altered by specific or overall changes in levels of polyamines. For example, specific polyamine concentrations can mediate different outcomes for microbial biofilm production. In multiple species, it has been demonstrated that intracellular polyamine levels regulate biofilm biogenesis and that this regulation is likely due to intracellular sensing of specific polyamines. Karatan et al. (2013) Biotechnol Lett. 35(11):1715-7; McGinnis et al. (2009) FEMS Microbiol Lett. 299(2):166-74; Wortham et al. (2010) Environ Microbiol. 12(7):2034-47. Which polyamines mediate these phenotypes can be species-specific. In mutant strains lacking the ability to produce a specific polyamine, exogenous addition of that polyamine that is unable to be synthesized restored biofilm formation, while addition of other polyamines did not. Additionally, the literature is replete with examples of bacterial biofilm formation being inhibited by endogenous or high concentrations of exogenous specific polyamines and their derivatives, while other polyamines have no effect. Karatan et al. (2013) Biotechnol Lett. 35(11):1715-7; Goytia et al. (2013) FEMS Microbiol Lett. 343(1):64-9; Cardile et al. (2017) Adv Exp Med Biol. 973:53-70; Wang et al. (2016) J Bacteriol. 198(19):2682-91; Qu et al. (2016) Microbiologyopen. 5(3):402-12; Konai et al. (2015) Bioconjug Chem. 26(12):2442-53; Si et al. (2015) Appl Microbiol Biotechnol. 99(24):10861-70; Dewangan et al. (2014) Antimicrob Agents Chemother. 58(9):5435-47; Ding et al. (2014) Appl Environ Microbiol. 80(4):1498-506; Planet et al. (2013) MBio. 4(6):e00889-13. However, the same polyamines that inhibit one species may not inhibit biofilm biogenesis and may even be required for biofilm production in other bacterial species. Karatan et al. (2013) Biotechnol Lett. 35(11):1715-7; McGinnis et al. (2009) FEMS Microbiol Lett. 299(2):166-74; Wortham et al. (2010) Environ Microbiol. 12(7):2034-47; Wang et al. (2016) J Bacteriol. 198(19):2682-91; Hobley et al. (2017) J Biol Chem. 292(29):12041-53; Ou et al. (2017) Mol Med Rep. 15(1):21-20; Nesse et al. (2015) Appl Environ Microbiol. 81(6):2226-32; Ramon-Perez et al. (2015) Microb Pathog. 79:8-16; Hobley et al. (2014) Cell. 156(4):844-54; Sakamoto et al. (2012) Int J Biochem Cell Biol. 44(11):1877-86; Burrell et al. (2010) J Biol Chem. 285(50):39224-38; Lee et al. (2009) J Biol Chem. 284(15):9899-907; Patel et al. (2006) J Bacteriol. 188(7):2355-63. Furthermore, while the role of polyamines in fungal biofilm development is less well defined, Candida albicans mutants in polyamine synthesis are defective for biofilm production and treatment of C. albicans with polyamine synthesis inhibitors negatively affects biofilm growth. Chen et al. (2014) Mol Biosyst. 10(1):74-85; Liao et al. (2015) Int J Antimicrob Agents. 46(1):45-52.

One potential source of eDNA stabilization is the presence of polyamines in the biofilm matrix. Polyamines have been observed to modulate DNA structure (Pasini et al. (2014) Amino Acids. 46(3):595-603) and protect DNA from external modifying agents or hazardous conditions. D'Agostino et al. (2005) FEBS J. 272(15):3777-87; Baeza et al. (1991) Orig Life Evol Biosph. 21(4):225-42; Nayvelt et al. (2010) Biomacromolecules. 11(1):97-105. The role of polyamines in intracellular chromatin stabilization has been well-documented. Pasini et al. (2014) Amino Acids. 46(3):595-603. Here, Applicants hypothesized that extracellular polyamines stabilize the eDNA structure of bacterial biofilms and show that altering the polyamine content and the ability of polyamines to bind eDNA in the biofilm matrix extracellularly as a means to disrupt bacterial biofilm communities. Applicants observed that inhibition of synthesis or antagonism of polyamines disrupted established bacterial biofilms and that supplementation of polyamine-depleted bacteria restored eDNA structure.

### Polyamines are Present in the Extracellular Matrix of Bacterial Biofilms

To determine whether polyamines were present in the biofilm matrix, as a model human pathogen, Applicants grew non-typeable Haemophilus influenzae (NTHi) biofilms in vitro and performed immunofluorescence with antibodies directed towards putrescine, spermine, or spermidine. Polyamine localization within biofilm extracellular matrix was visualized using confocal laser scanning microscopy (CLSM; FIG. 12). All three polyamines were detected by immunofluorescence microscopy throughout the biofilm.

### Polyamine Synthesis Inhibition or Polyamine Antagonism Disrupts Bacterial Biofilms

Dicyclohexylamine inhibits spermidine synthase via a competitive inhibition mechanism, i.e. through binding to the same site on the protein as the putrescine substrate. Applicants therefore hypothesized that dicyclohexylamine would inhibit NTHi biofilm development. First, Applicants confirmed that dicyclohexylamine did not affect NTHi growth up to 10 mM (FIG. 13). However, addition of 50 μM dicyclohexylamine significantly inhibited NTHi biofilm formation as evaluated by COMSTAT analysis, reducing biofilm thickness and biomass by approximately 40% while increasing biofilm roughness (FIG. 14). Subsequent immunofluorescence microscopy for the presence of spermidine in the residual NTHi biofilms treated with dicyclohexylamine revealed a statistically significant corresponding decrease in spermidine present in the biofilm matrix (FIG. 15). Dicyclohexylamine also reduced the amount and the complexity of eDNA scaffold structures produced during early biofilm formation (FIG. 16). Exogenous addition of 1 mM spermidine to dicyclohexylamine-treated NTHi biofilms restored normal biofilm thickness, biomass, and roughness (FIG. 14), indicating that dicyclohexylamine was functioning as a competitive inhibitor of either spermidine synthase and or exogenous spermidine in the biofilm extracellular matrix. These data reveal that, through inhibition of polyamine synthesis or antagonistic binding to polyamine binding sites, compounds directed at extracellular polyamines binding to eDNA in the biofilm matrix have potential for prevention of biofilm biogenesis and disruption of established biofilms.

### Experiment No. 2

It is estimated that the pathogenesis of >80% of all bacterial infectious diseases include a necessary biofilm state in the pathogenesis of the disease course, according to the Centers for Disease Control and Prevention. Biofilms are comprised of bacterial cells attached to abiotic and biotic surfaces that have progressed into a structured population that is embedded within an extracellular polymeric substance (EPS) that includes nucleic acids, proteins, lipids, biopolymers (Davies (2003) Nat Rev Drug Discov. 2(2):114-22), and divalent cations (Cavaliere et al., (2014) Microbiology Open. 3(4):557-567). The EPS acts as a protective barrier against harsh environments and antimicrobial agents such as antibiotics and host immune effectors (Devaraj et al., (2013), supra. Crucial structural and architectural components of the biofilm matrix are extracellular DNA (eDNA) and the DNABII family of DNA-binding proteins (IHF and HU). DNABII proteins bind with high affinity to eDNA which permits stabilization of the biofilm. Antibodies targeting DNABII induce collapse of the biofilm with release of the resident bacteria in vitro and in vivo (Novotny et al. (2016) EBioMedicine. (10):33-44); and Goodman et al. (2011) Mucosal Immunology. 4 (6): 625-637. While DNase treatment can prevent bacterial species from forming a biofilm, it has little to no effect on pre-existing biofilms (Flemming and Wingender, (2010) Nature Reviews Microbiology. 8:623-633. Positively charged divalent cations (Mg²⁺, Mn²⁺, Zn²⁺, Cu²⁺ and Ca²⁺) mediate intermolecular cross-linking between adjacent negatively charged DNA molecules. This interaction stabilizes the DNA structure and subsequent DNA-protein interactions (Gueroult et al. (2012) PLOS ONE. (7)-7-e41704; and Tan and Chen, (2006) Biophysical Journal. (90): 1175-1190; Hackl et al. (2005) International Journal of Biological Macromolecules 35:175-191. Furthermore, removal of Mg²⁺ cations from biofilms increases the susceptibility of nontypeable Haemophilus influenzae (NTHi) to antibiotic treatment (Cavaliere et al. (2014) Microbiology Open. 3(4):557-567. Finally, polyamines, (short polycationic biogenic amines) are also important for biofilm formation by multiple bacterial species (Patel et al. (2006) Journal of Bacteriology. 2355-2363; and Hobley et al., (2017) Journal of Biological Chemistry. 292(29): 12041-12053. Immunofluorescence CLSM (IF) images of biofilms formed by many pathogenic bacteria when probed for the presence of spermidine indicate that polyamines are part of the EPS of bacterial biofilms and further, that they co-localize with the DNABII protein HU (FIG. 17A). Inhibition of the spermidine biosynthesis pathway in NTHI by the enzyme inhibitor dicyclohexylamine (DCHA) results in an overall decrease in spermidine levels as measured by IF and a significant decrease in biofilm average thickness, thus indicating polyamines are critical to the stability of biofilms (FIG. 17B and FIG. 17C).

The ability of microorganisms to form biofilms is highly problematic and ubiquitous among a broad range of industries. For instance, hospital acquired device-associated infections of mechanical heart valves, urinary catheters, and venous catheters are the result of bacterial contamination in the form of a biofilm (Donlan, (2001) Emerging Infectious Diseases. 7(2):277-281. Controlling biofilm formation in agriculture and food processing facilities is also important for prevention of disease and extensive food loss. Chmielewski and Frank (2006) Compr Rev Food Sci F 2:22-32. Wastewater treatment facilities also develop biofilm-mediated issues such as biofouling, the accumulation of EPS and microorganisms that prevent proper membrane filtration, which leads to water contamination (Wood et al., (2016) PNAS. E2802-E2811).

Coating surfaces and/or treating biofilms with cation exchange resins (sulfonate, sulfopropyl, phosphocellulose, or heparin sepharose) utilizes the properties of negatively charged resin chemistry to target positively charged components of the EPS (i.e. polyamines, divalent metal cations and DNABII proteins). This results in biofilm disruption and prevention on both biotic and abiotic surfaces. Here Applicants demonstrate that the cation exchange resins P11 phosphocellulose and heparin sepharose prevent biofilm formation and are able to disrupt pre-formed biofilms.

Cation Exchange Resins have a Negative Effect on Preformed Biofilms and Biofilm Formation by Nontypeable Haemophilus influenzae (NTHI).

Applicants questioned if negatively charged resins incapable of penetrating biofilms could act to titrate out positively charge molecules (i.e. polyamines, divalent metal cations and DNABII proteins) that are universally required for bacterial biofilm formation. Two resins were chosen, e.g., phosphocellulose (P11) and heparin sepharose, both of which are cation exchangers used for ion exchange chromatography, but also used for affinity purification of DNABII proteins (Nash et al. (1987) Journal of Bacteriology. 4124-4127; and Vorgias and Wilson, (1991) Escherichia coli. Protein Expression and Purification. 2(5-6):317-20).

To determine anti-biofilm activity of cation exchange resins on preformed biofilms (i.e. ability to disrupt an extant biofilm), NTHI growth was initiated and maintained for 24 hrs, then treated for 16 hrs with 0 (sBHI Control), 0.1, 1, or 5% (w/v) of P11 phosphocellulose (FIG. 18A). To determine the anti-biofilm activity of cation exchange resins to prevent biofilm formation, NTHI growth was initiated and maintained in the presence of 0 (sBHI Control), 0.1, 1, and 5% w/v of P11 phosphocellulose (FIG. 18B) or 5% (w/v) heparin sepharose (FIG. 19). Biofilms were washed with saline and stained with LIVE/DEAD®, visualized with confocal scanning microscopy (CSLM) and analyzed by COMSTAT to determine average thickness and biomass. As indicated in FIG. 18, P11 phosphocellulose had a negative effect on both preformed biofilms (was able to disrupt) and biofilm formation (was able to prevent) which was revealed by the decrease in average thickness and biomass. Heparin sepharose also had a negative effect on biofilm formation where a decrease in average thickness and biomass was observed (FIG. 18). These data suggest that cation exchange resins can both disrupt and prevent biofilm formation in vitro.

### DNABII (HU) Partially Restored Cation-Depleted Preformed NTHi Biofilms.

To determine whether removal of DNABII proteins was in part responsible for the observed biofilm disruption and prevention, NTHI growth was initiated and maintained in the presence of 0 (sBHI control) or 1% P11 phosphocellulose (w/v) for 24 hrs with the exogenous addition of HU protein at 1 or 5 ug/mL for 16 hrs. Biofilms were washed with saline and stained with LIVE/DEAD®, visualized with CSLM and analyzed by COMSTAT to determine average thickness and biomass. As shown in FIG. 19, HU can partially compensate for the negative effect of phosphocellulose in vitro. These data suggest HU is targeted by the cation exchange resin.

### Divalent Metals Partially Restore Cation-Depleted Preformed NTHI Biofilms.

To determine whether Mg′ can restore cation depleted biofilms, NTHI growth was initiated and maintained in the presence of 0 (sBHI Control) or 1% P11 phosphocellulose (w/v) for 24 hrs with exogenous addition of MgCl₂ (0-10 mM) for 16 hrs. Biofilms were washed with saline and stained with LIVE/DEAD®, visualized with CSLM and analyzed by COMSTAT to determine average thickness and biomass. As shown in FIG. 20, MgCl₂ can partially compensate for the disruptive effect of phosphocellulose in vitro. These data suggest that P11 phosphocellulose depletes divalent metals from the biofilm matrix.

### Spermidine Partially Restore Cation Depleted Preformed NTHI Biofilms

To determine whether spermidine can restore cation-depleted biofilms, NTHI growth was initiated and maintained in the presence of 0 (sBHI Control) or 1% P11 phosphocellulose (w/v) for 24 hrs with exogenous addition of spermidine (0-5 mM) for 16 hrs. Biofilms were washed with saline and stained with LIVE/DEAD®, visualized with CSLM and analyzed by COMSTAT to determine average thickness and biomass. As shown in FIG. 21, spermidine can partially compensate for the negative effect of phosphocellulose in vitro. These data suggest that P11 phosphocellulose depletes polyamines from the biofilm matrix.

Cation Depletion Effects of P11 Phosphocellulose does not Require Direct Contact with Biofilm

To determine whether the decrease in biofilm formation was dependent on direct cellular contact, NTHI growth was initiated in the basal chamber of a transwell plate system that contained a 0.4 μm pore size within the membrane that separates the apical and basolateral chambers. This allows for the diffusion of small molecules e.g. proteins (DNABII), polyamines and divalent metal cations between the two chambers, but not bacterial cells. The apical chamber contained 0 (sBHI Control), 0.5, 1, or 1.5% (w/v) P11 phosphocellulose at seeding and maintained for 16 hrs. Biofilms were washed with saline and stained with LIVE/DEAD®, visualized with CSLM and analyzed by COMSTAT to determine average thickness and biomass. As indicated in FIG. 22, the dose-dependent decrease in biofilm average thickness and biomass is independent of direct contact with the cation exchange resin.

### Exogenous Addition of Spermidine Compensates for the Cation Depletion Effects of P11 Phosphocellulose in the Transwell System

To determine whether polyamines (spermidine) are depleted by P11 phosphocellulose without direct contact in the transwell system, NTHi growth was initiated in the basolateral chamber containing 0, 100, 500, or 1000 μM Spermidine, while 0 (sBHI control) or 1.5% (w/v) P11 phosphocellulose was added to the apical chamber and maintained for 16 hrs. Biofilms were washed with saline and stained with LIVE/DEAD®, visualized with CSLM and analyzed by COMSTAT to determine average thickness and biomass. As indicated in FIG. 23, spermidine alone can restore cation-depleted biofilms in a dose-dependent manner.

### Spermidine and DNABII Act Synergistically to Restore Non-Contact Mediated P11 Phosphocellulose Cation-Depleted Biofilms

P11 phosphocellulose cation-depleted biofilms can be restored by the exogenous addition of HU (FIG. 20) and spermidine (FIG. 22 and FIG. 24) in a dose-dependent manner. As shown in FIG. 17A, immunofluorescence CSLM indicates that spermidine and DNABII (HU) also co-localize in the biofilms formed by multiple pathogenic bacterial species. Thereby, Applicants wanted to determine whether HU and spermidine act synergistically in vitro. NTHi growth was initiated in the basolateral chamber of the transwell system containing either 100 μM spermidine or 500 nM HU, or a combination of both components. While, 0 or 1.5% P11 phosphocellulose was added to the apical chamber and maintained for 16 hrs. Biofilms were washed with saline and stained with LIVE/DEAD®, visualized with CSLM and analyzed by COMSTAT to determine average thickness and biomass. As indicated in FIG. 25, the combination of suboptimal concentrations of both spermidine and HU restore cation-depleted biofilms in a synergistic manner, compared to the sBHI control biofilm.

Abiotic Surfaces Coated with Cation Exchange Resin Prevent Biofilm Formation in a Dose-Dependent Manner

Glass chamber slides were coated with solutions of 0, 0.1, 1, or 5% P11 phosphocellulose and 5% heparin sepharose (w/v). NTHI growth was initiated and maintained for 40 hrs on coated slides. Biofilms were washed with saline and stained with LIVE/DEAD®, visualized with CSLM and analyzed by COMSTAT to determine average thickness and biomass. FIG. 26 indicates that both P11 phosphocellulose and heparin sepharose prevent biofilm formation in a dose dependent manner. These data suggest that coating surfaces with cation exchange resin can prevent bacterial attachment and subsequent biofilm formation in vitro.

### Experiment No. 3

Biofilms consist of communities of microorganisms of exclusively bacteria, exclusively yeast or both. For bacterial pathogens, antibiotics are the first line of treatment. Bacteria resident within a biofilm contributes significantly to the pathogenesis of approximately 80% of all bacterial infections and is known to contribute to the chronic and recurrent nature of infectious diseases. Also, bacteria resident within a biofilm are up to a 1000-fold more resistant to antibiotics than are their free-living counterparts. This resistance is owed mostly to an extracellular matrix that protects the resident microorganisms from a hostile environment. The chronic and recurrent nature of biofilm-mediated bacterial infections demand excessive use of antibiotics that in turn, has led to the sobering emergence of multiple antibiotic-resistant bacteria globally. This growing antibiotic resistance phenotype results in failure of antibiotic therapy. Also, the major side effect of antibiotics is that they negatively impact the commensal microbiota, which can leave the host with any of multiple side effects as well as susceptibility to secondary infections. Previously Applicants identified DNA and the DNABII family of proteins as universal targets within all bacterial biofilm matrices studied to date. Applicants showed that the DNABII proteins can be removed with therapeutic antibodies resulting in the structural collapse of the biofilm and release of the resident bacteria. In contrast, yeast biofilms do not express DNABII proteins but still contain extracellular DNA (eDNA) which is also part of their matrix. This new approach is an improvement over current therapeutic technologies in that it does not rely upon compounds with bactericidal activities, which apply pressure on the bacteria to develop resistance mechanisms, but rather targets the biofilm extracellular matrix structure itself, thereby resulting in disruption of the biofilm and release of resident bacteria into a vulnerable, accessible state. All biofilms, regardless of constituent bacteria or yeast, create DNA-dependent structures but rather than these structures existing as B-DNA (the canonical form that predominates intracellularly), the most important structural eDNA present within a biofilm matrix exists as Z-DNA. Importantly, Z-DNA is more rigid, making it a better structural material and Z-DNA is completely resistant to nucleases, enzymes that degrade B-DNA. Hence, the target of any potential therapies is a previously unrecognized structural element of the biofilm matrix that when altered (i.e. transitioned back to B-DNA) or disrupted, would potentiate the efficacy of current therapies. Importantly, proteins that bind Z-DNA also stabilize Z-DNA. Since Z-DNA is a natural constituent of biofilms, antibodies that bind to Z-DNA will facilitate biofilm growth. Conversely, molecules that bind B DNA stabilize B DNA and will prevent the conversion of B-DNA into Z-DNA and thus prevent biofilms. Importantly, disease states that naturally induce the formation of antibodies directed against Z-DNA will facilitate biofilm formation e.g. Systemic Lupus Erythematosus (SLE) and/or cystic fibrosis (CF). Thus chronic infections that develop in SLE patients are, in part, the result of the SLE derived Z-DNA antibodies. In addition, some chemotherapeutic agents that damage DNA e.g. platinum based chemotherapies, also shift DNA to the Z-form. Hence these agents contribute to stabilizing or facilitating biofilm growth. Directing therapeutics that disrupt Z-DNA is a new therapeutic approach for SLE and/or cystic fibrosis (CF) and chemotherapeutic exacerbations of chronic infections.

Biofilms are a collection of microorganisms aggregated or adhered to a surface that display community architecture and behavior (intracommunity signaling, transport, division of labor, etc.). This community architecture is in part distinguished by a self-made extracellular matrix that protects the resident microorganisms against hazardous conditions, which in a host includes the immune system and antimicrobials. Biofilms are responsible for a significant portion of disease, in both animals and plants, as well as industrially e.g. in fouling of industrial equipment, and as such, are the focus of intense research efforts due to their importance in medical, agricultural, and industrial settings. Visick et al. (2016) J Bacteriol. 198(19):2553-63; Hoiby et al. (2017) APMIS. 125(4):272-5. Eradication or treatment of pathogenic biofilms is particularly difficult to accomplish due to multiple factors, including production of the protective extracellular matrix. Hoiby et al. (2017) APMIS. 125(4):272-5. The biofilm matrix is variably comprised of polysaccharides, proteins, and extracellular DNA (eDNA). The eDNA of bacterial biofilms is universal and essential for the stability and protective functions of the extracellular matrix. Okshevsky et al. (2015) Crit Rev Microbiol. 41(3):341-52; Wilton et al. (2015) Antimicrob Agents Chemother. 60(1):544-53. Undermining the biofilm eDNA structure, via DNA degradation or removal of DNA binding proteins that stabilize the structure, results in catastrophic collapse of the biofilm and release of the resident bacteria into a more vulnerable state. Brandstetter et al. (2013) Nasopore. Laryngoscope. 123(11):2626-32; Brockson et al. (2014) Mol Microbiol. 93(6):1246-58; Devaraj et al. (2015) Mol Microbiol. 96(6):1119-35; Freire et al. (2017) Mol Oral Microbiol. 32(1):74; Gustave et al. (2013) J Cyst Fibros. 12(4):384-9; Novotny et al. (2017) Clin Vaccine Immunol. 24(6); Novotny et al. (2016) EBioMedicine. 10:33-44; Rocco et al. (2017) Mol Oral Microbiol. 32(2):118-30; Novotny et al. (2013) PLoS One. 8(6):e67629; Baelo et al. (2015) J Control Release. 209:150-8; Brown et al. (2015) Front Microbiol. 6:699; Frederiksen et al. (2006) Acta Paediatr. 95(9):1070-4; Martins et al. (2012) Mycoses. 55(1):80-5; Goodman et al. (2011) Mucosal Immunol. 4(6):625-37.

Based on the central role of eDNA in biofilm integrity, nuclease treatment carries obvious therapeutic potential. Indeed, many bacteria utilize endogenous secreted nucleases to modulate biofilm structure and mediate dispersal from biofilms. Cho et al. (2015) Infect Immun. 83(3):950-7; Kiedrowski et al. (2011) PLoS One. 6(11):e26714; Liu et al. (2017) Front Cell Infect Microbiol. 7:97; Steichen et al. (2011) Infect Immun. 79(4):1504-11. However, exogenous nucleases typically only show effective biofilm prevention activity when administered at the time of initiation of biofilm formation, with established biofilms requiring high concentrations of nuclease to observe any modest biofilm disruption. Hall-Stoodley et al. (2008) BMC Microbiol. 8:173; Izano et al. (2009) Microb Pathog. 46(4):207-13; Kaplan et al. (2012) J Antibiot (Tokyo). 65(2):73-7; Tetz et al. (2010) DNA Cell Biol. 29(8):399-405. In cystic fibrosis patients, where chronic pulmonary infections are the primary source of morbidity and mortality, therapeutic nucleases are primarily used as mucolytic agents targeted towards obstructions created by host-derived eDNA rather than the eDNA produced by bacteria to form a biofilm community, and these nucleases (i.e. Pulmozyme) have only been observed to alter microbial communities when administered very early in life prior to establishment of chronic infections. Frederiksen et al. (2006) Acta Paediatr. 95(9):1070-4. Why bacterial biofilm eDNA becomes resistant to nuclease degradation has remained an open question.

Multiple hypotheses exist for why bacterial biofilm eDNA is insufficiently degraded by exogenous nucleases. Among these hypotheses is the possibility that bacteria actively alter the structure of the eDNA to a form that is insensitive to nucleases. One such DNA structural element that is nuclease resistant is Z-DNA, an alternative left-handed helical form. Z-DNA has only been described under specific conditions in vitro and limited intracellular conditions in vivo, though evidence is mounting for a role for Z-DNA in normal cellular physiology, particularly with the identification of Z-DNA binding proteins that regulate various intracellular processes. Wang et al. (2007) Front Biosci. 12:4424-38; Barraud et al. (2012) Curr Top Microbiol Immunol. 353:35-60. In order to shift the equilibrium from the canonical B-DNA to the Z-DNA form, additional factors are required (Choi et al. (2011) Chem Soc Rev. 40(12):5893-909; Yang, et al. (2012) Curr Med Chem. 2012; 19(4):557-68), including negative supercoiling of the double helix, high cationic concentrations to counteract the repulsion of the negatively charged phosphate-deoxyribose backbone, and Z-DNA binding protein interactions to stabilize the structure. Metal cations and polycationic polyamines are both capable of inducing Z-DNA formation (D'Agostino et al. (2006) IUBMB Life. 58(2):75-82; Balasundaram et al. (1991) Mol Cell Biochem. 100(2):129-40), particularly in poly(dG-dC) tracts. Thomas et al. (1991) J Biol Chem. 266(10):6137-41. On the other hand, Z-DNA transition back to B-DNA is catalyzed by intercalating agents (Kim et al. (1993) Biopolymers. 33(11):1677-86; Mirau et al. (1983) Nucleic Acids Res. 11(6):1931-41) and, potentially, the eukaryotic chromatin protein, HMGB1. Waga et al. (1988) Biochem Biophys Res Commun. 153(1):334-9.

The biofilm matrix is stabilized by bacterial chromatin proteins of the DNABII family. These proteins occupy bent DNA structures in the eDNA scaffold, and their removal results in collapse of the biofilm and release of resident bacteria. Brandstetter et al. (2013) Nasopore. Laryngoscope. 123(11):2626-32; Brockson et al. (2014) Mol Microbiol. 93(6):1246-58; Devaraj et al. (2015) Mol Microbiol. 96(6):1119-35; Freire et al. (2017) Mol Oral Microbiol. 32(1):74-88; Gustave et al. (2013) J Cyst Fibros. 12(4):384-9; Novotny et al. (2017) Clin Vaccine Immunol. 24(6); Novotny et al. (2016) EBioMedicine. 10:33-44; Rocco et al. (2017) Mol Oral Microbiol. 32(2):118-30; Novotny et al. (2013) PLoS One. 8(6):e67629; Goodman et al. (2011) Mucosal Immunol. 4(6):625-37. Additionally, polyamines have been observed to modulate DNA structure (Pasini et al. (2014) Amino Acids. 46(3):595-603) and protect it from external modifying agents. Baeza et al. (1991) Orig Life Evol Biosph. 21(4):225-42; D'Agostino et al. (2005) FEBS J. 272(15):3777-87; Nayvelt et al. (2010) Biomacromolecules. 11(1):97-105. Here, Applicants show that extracellular polyamines in collaboration with DNABII proteins induce Z-DNA structure in eDNA within bacterial biofilms and establish whether reversion of Z-DNA to B-DNA would undermine bacterial biofilm structure to either make it susceptible to conventional therapeutics or, in and of itself, disrupt biofilms.

### Bacterial Biofilms Become Resistant to Nuclease Disruption as Z-DNA Accumulates

Non-typeable Haemophilus influenzae (NTHi) is a common cause of acute and chronic respiratory tract infections, and disease caused by this organism relies on biofilm formation for its chronicity. Duell et al. (2016) FEBS Lett. 590(21):3840-53. Similarly, uropathogenic Escherichia coli (UPEC) is the primary etiological agent of urinary tract infections and also relies on biofilm production for its ability to cause chronic cystitis. Blango et al. (2010) Antimicrob Agents Chemother. 54(5):1855-63. NTHi and UPEC biofilms grown in vitro in the absence or presence of nucleases showed significant impairment for biofilm biogenesis when nucleases were present (FIG. 27). In contrast, treatment of pre-established biofilms with these nucleases did not diminish biofilm thickness or biomass, indicating that resistance of the eDNA in the biofilm matrix to nucleases is established as the biofilm matures.

Applicants used an antibody directed against Z-DNA to probe NTHi and UPEC biofilms in vitro as they developed. Antibody staining and immunofluorescence microscopy revealed that Z-DNA accumulates at the base of the biofilm matrix as the biofilm matures (FIG. 28), corresponding with increased nuclease resistance in mature biofilms. The major fungal pathogen Candida albicans was also found to contain Z-DNA within its biofilm extracellular matrix (FIG. 29). Furthermore, the accumulation of Z-DNA is central to the biofilm-building process, as treatment of nascent biofilms with antibodies that specifically bind (and therefore stabilize) Z-DNA stimulated NTHi biofilm biogenesis, while antibodies directed towards B-DNA did not (FIG. 30). Z-DNA likely confers resistance to nuclease degradation to the biofilm extracellular matrix, which is demonstrated by examining the eDNA content and forms of DNA present in biofilms treated with nucleases. Nuclease treatment greatly reduces B-DNA visualization in the NTHi biofilm extracellular matrix, while Z-DNA presence becomes more apparent (FIG. 31).

### Polyamines and DNABII Proteins Cooperate to Stabilize Z-DNA Structures in the Biofilm Matrix

An oligonucleotide substrate comprised of poly(dG-dC) is known to be prone to form Z-DNA (i.e. due to high NaCl concentrations or presence of polyamines) and as such, is protected from degradation by nucleases (FIG. 32). Accordingly, Applicants also observed that polyamines induce Z-DNA formation of this substrate via spectrophotometric measurement of the X295/X260 absorbance ratio (data not shown). In addition to polyamine-mediated eDNA stabilization, Applicants already have demonstrated the importance of DNABII bacterial chromatin proteins in reinforcing the biofilm structure. Now, Applicants sought to determine whether polyamines and DNABII proteins function together to stabilize the eDNA scaffold. Using an antibody directed towards two related polyamines, spermidine and spermine, and antibodies recognizing the two DNABII family proteins, IHF and HU, Applicants first visualized the distribution of polyamines and the DNABII proteins in the biofilm matrix by immunofluorescence microscopy. Applicants found that DNABII proteins (primarily HU) co-localized with polyamines within the NTHi biofilm extracellular matrix (FIG. 33). Furthermore, immunofluorescence microscopy of biofilms formed by an NTHi strain engineered to be unable to express HU and probed with the polyamine-specific antibody demonstrated that HU is required for normal polyamine distribution in the biofilm (FIG. 33).

Since NTHi DNABII proteins play a central role in biofilm matrix integrity. Brockson et al. (2014) Mol Microbiol. 93(6):1246-58; Novotny et al. (2017) Clin Vaccine Immunol. 24(6); Novotny et al. (2016) EBioMedicine. 10:33-44; Goodman et al. (2011) Mucosal Immunol. 4(6):625-37, Applicants postulated that they may contribute to DNA nuclease resistance. Indeed, NTHi HU was capable of conferring nuclease resistance to a poly(dG-dC) substrate in vitro and of shifting a poly(dGdC) substrate to the Z-DNA form (FIG. 34) Jang et al. (2015) Sci Rep. 5:9943. Due to the observation of co-localization of NTHi HU and polyamines in the biofilm matrix, Applicants tested whether they displayed synergy in their ability to inhibit nuclease degradation. At concentrations of polyamines and HU that did not protect the poly(dG-dC) substrate on their own, combination of the two demonstrated synergistic ability to confer nuclease resistance (FIG. 35). The co-localization of polyamines and DNABII proteins, and therefore the resultant nuclease resistant phenotype, was also observed in biofilms formed in vivo in the middle ear during experimental otitis media due to NTHI (FIG. 35). Furthermore, the levels of DNABII and polyamine incorporation into the biofilm extracellular matrix varies among species and is correlated with the level of Z-DNA detection by immunofluorescence (FIG. 36). Together, these results suggest that bacterial DNABII proteins and endogenous polyamines interact together to maintain the bacterial biofilm matrix, potentially via conversion to and stabilization of Z-DNA structural elements within the eDNA scaffold of biofilms.

### Reversion of Z-DNA to B-DNA Restores Nuclease Sensitivity

In order to take advantage of Applicants' novel findings that Z-DNA accumulates over time in bacterial biofilms and that this may explain why mature biofilms are resistant to nuclease disruption, Applicants sought to test whether molecules capable of shifting the equilibrium of the Z-DNA to B-DNA could modulate biofilms. The eukaryotic chromatin protein HMGB1 has been reported to convert Z-DNA to B-DNA Waga et al. (1988) Biochem Biophys Res Commun. 153(1):334-9. As such, immunofluorescence microscopy of NTHi biofilms treated with HMGB1 revealed diminished Z-DNA content (FIG. 37). DNABII proteins were released from the biofilm by HMGB1 treatment (FIG. 38), destabilizing the eDNA structure by allowing Z-DNA to revert to B-DNA in the absence of DNABII-mediated Z-DNA stabilization and resulting in significant biofilm disruption. Accordingly, treatment of acute otitis media induced by NTHI in the chinchilla middle ear with HMGB1 or a non-inflammatory HMGB1 variant resulted in efficient resolution of adherent biofilms (FIG. 39). HMGB1 was also effective at disrupting biofilms formed in vitro by Burkholderia cenocepacia, a deadly opportunistic pathogen associated with cystic fibrosis pulmonary biofilm infections, while prophylactic administration of HMGB1 prevented B. cenocepacia aggregate biofilm formation in the lungs of mice (FIG. 40). Addition of HMGB1 to the poly(dG-dC) substrate in the presence of protective concentrations of polyamines restored nuclease sensitivity (FIG. 41).

These combined data demonstrate that conversion of a portion of the DNA into Z-conformation by polyamines and DNABII proteins not only creates the basis for the structural material in extracellular matrix of biofilms but explains its resistance to conventional nucleases to disrupt mature biofilms. Cho et al. (2015) Infect Immun. 83(3):950-7. Together, these data therefore indicate that methods and substances that can convert Z-DNA to B-DNA or biofilm eDNA in the Z-DNA configuration to B-DNA (e.g. by removing or inhibiting DNABII proteins or polyamines) can restore nuclease sensitivity, potentially allowing for development of agents that can potentiate the activity of nucleases against mature, recalcitrant biofilms in vivo. This also shows that microbial biofilms can both be identified by their Z-DNA content, making Z-DNA quantification a potentially useful biomarker diagnostic for biofilm infection.

### Experiment No. 4

This experiment provides a porcine model for pre-clinical testing of drugs, agents and methods to treat cystic fibrosis. See Stoltz et al. (2010) Science Translational Medicine 2(29):29-31. Cystic fibrosis is an autosomal recessive disease due to mutations in a gene that encodes the CF transmembrane conductance regulator (called CFTR) anion channel. In this model, pigs which have been specifically bred to carry a defect in the genes called “CFTR” and called CF pigs spontaneously develop hallmark features of CF lung disease that includes infection of the lower airway by multiple bacterial species. The pigs are immunized with the agents such as polypeptides or other immunogenic agents thereby inducing the formation of antibodies which will eradicate bacterial biofilms in the lungs. This Experiment is similar to delivering antibodies to IHF to eradicate biofilms resident within the middle ears of chinchillas following active immunization as shown in Experiment No. 1. The anti-IHF (or other agent) antibodies can be delivered to the lungs of these pigs by nebulization to assess the amelioration of the signs of disease and associated pathologies.

### Experiment No. 5

Identify UPECUPEC Polyamine Synthesis Genes that Contribute to Formation of the TEDS.

Using the E. coli Keio Collection of single mutants, Baba et al. (2006) Mol Syst Biol. 2:2006 0008, mutations are moved by P1 phage transduction of speA (arginine decarboxylase required to convert arginine to the putrescine precursor, agmatine), speC (ornithine decarboxylase required to convert ornithine to putrescine), speD (adenosylmethionine decarboxylase required to convert S-adenosylmethionine into the propylamine donor for SpeE activity), and speE (aminopropyltransferase required to convert putrescine to spermidine and spermidine to spermine) genes, Tabor et al. (1985) Microbiol Rev. 49(1):81-99, into the UPEC UTI89 strain, first singly, then in combination. Due to multiple pathways capable of producing polyamines, these mutations can be combined to observe biofilm phenotypes (e.g. SpeA and SpeD are the initial enzymes in two separate pathways of putrescine biosynthesis; a double mutation in speA and speD is needed to abrogate putrescine synthesis). Mutations are confirmed by PCR for insertion at the correct chromosomal location. The UPEC strain is established in Applicants' in vitro eDNA scaffold assay and biofilm formation assay at defined times (8, 24, 48 h) and in the presence or absence of an added polyamine (spermidine, spermine, or putrescine) at physiologic concentrations (0, 0.1, 0.5, 1, and 5 mM). Tabor et al. (1985) Microbiol Rev. 49(1):81-99. These assays are performed in both a rich medium (LB) and a chemically defined medium (CDM; M9) as there are likely residual polyamines in rich media. Initial eDNA structure are evaluated by immunofluorescence microscopy probed for dsDNA, DNABII proteins, and polyamines (anti-spermidine, anti-putrescine, anti-cadaverine), and complexity of structures is quantified by FracLac analysis plugin in ImageJ. Karperien et al. (1999-2013) FracLac for ImageJ. Biofilm formation is evaluated by CLSM of LIVE/DEAD®-stained biofilms and biofilm average thickness, biomass, and roughness is quantified by COMSTAT analysis. eDNA structure in biofilms probed for dsDNA, DNABII proteins, and polyamines are evaluated by immunofluorescence CLSM. Anti-HU antibodies directed against a conserved epitope are used, enabling comprehensive recognition of HU across genera for DNABII detection, since all eubacteria have HU, and HU depletion universally results in biofilm impairment. FIG. 46 shows Immunofluorescence CLSM images of indicated biofilms probed with naive or anti-HU (gray) and anti-spermidine (Spd, light gray) antibodies, co-localization is white.

Identify the UPEC Polyamine Export Genes that Contribute to Formation of the TEDS

Using the Keio Collection, mutations of potE (putrescine-ornithine antiporter), Kashiwagi et al. (1992) Proc Natl Acad Sci USA. 89(10):4529-33, cadB (cadaverine-lysine antiporter), Soksawatmaekhin et al. (2004) Mol Microbiol. 51(5):1401-12, mdtJ and mdtI (multidrug exporter that is important for spermidine export), Higashi et al. (2008) J Bacteriol. 190(3):872-8, and sapB, sapC, sapD, and sapF (encoding ABC transporter that is important for putrescine export) Sugiyama et al. (2016) J Biol Chem. 291(51):26343-51 genes are moved into UPEC UTI89, first singly and then in combination (to overcome redundancy of exporter activities). Mutations that display deficient biofilm phenotypes can be combined. UPEC is established in Applicants' eDNA scaffold and biofilm formation assays at defined times and in the presence or absence of an added polyamine (spermidine, spermine, putrescine).

The roles of polyamines in the TEDS and biofilm development and is determined by showing that the polyamines function in concert with eDNA and the DNABII proteins to create the TEDS. FIGS. 42A-42D show that both DNABII proteins and polyamines are required to rescue cation exchanger (P11)-mediated biofilm prevention. While FIG. 44 shows that DNABII proteins, polyamines, and eDNA (B- and Z-DNA) steadily accumulate within the EPS of NTHI biofilms over time. Furthermore, as shown in FIG. 46, DNABII, polyamines, Z-DNA and B-DNA components are all present in the EPS of multiple bacterial biofilms.

Applicants have also found in FIGS. 47A-B that polyamines, Z-DNA and B-DNA components are all present within sections of the chinchilla middle ear infected with NTHI biofilms. In addition, Applicants have shown that addition of RNase A stimulated biofilm formation in a dose-dependent manner, likely by the release of polyamines, a critical catalyst for the extracellular conversion of B-DNA to Z-DNA (FIG. 52A) and that addition of tRNA (but not GMP) disrupted early NTHI biofilms in a dose-dependent manner, likely by sequestration of polyamines from the biofilm EPS (FIG. 52B).

### Quantify Polyamines in Conditioned Medium.

Conditioned media is collected from bacterial biofilms to determine the concentrations of polyamines (putrescine, cadaverine, spermidine, spermine). Polyamine release is quantified for 8 recalcitrant biofilm-forming pathogens, hereafter referred to as the standard 8 strains; clinical isolates of the ES*KAPE pathogens, NTHI, and UPEC (*Indicates use of S. epidermidis as a representative pathogenic Staphylococcus Sabate et al. (2017) Front Microbiol. 8:1401, given that S. aureus produces antibody binding protein A that can confound immunofluorescence assays; where tractable, this analysis is applied to S. aureus) and mutant variants that show altered biofilm EPS structure to correlate biofilm phenotypes with extracellular polyamine concentrations. Biofilms are grown in rich media and CDM, and conditioned media collected at various times (0, 4, 8, 16 and 24 h). Cell free conditioned media (filtration) and media samples are snap frozen in liquid N₂ for storage. Analysis is conducted by the OARDC Metabolite Analysis Cluster via LC-MS/MS. Hakkinen et al. (2013) J Chromatogr B Analyt Technol Biomed Life Sci. 941:81-9; Liu et al. (2013) Anal Chim Acta. 791:36-45; Xu et al. (2016) Molecules. 21(8).

### Establish the Role of Steady State Levels of Polyamines in the TEDS.

After determining which polyamines are present throughout biofilm development in vitro, tests are conducted to distinguish if these polyamines affect eDNA structure. Applicants have identified a series of nucleases that prevent biofilm formation (similar or better than Pulmozyme®) when added at seeding but have varying sensitivities to spermidine (Table 1). Employing eDNA scaffold and biofilm formation assays over time, Applicants utilize these enzymes as qualitative probes for polyamine concentrations and to distinguish between polyamine-mediated inhibition of nuclease activity or eDNA structure-mediated inhibition of nuclease activity (i.e. conversion to a nuclease resistant form, like Z-DNA). In each case, biofilms are grown for specified times (0, 4, 8, 16, and 24 h) before treatment with nucleases and incubated to a final time of 40 h. As a control for nuclease activity in the biofilm environment, nuclease activity against a plasmid substrate incubated in conditioned media from the biofilms (0, 4, 8, 16 and 24 h) is determined, for which Applicants would have determined the polyamine constituents and respective concentrations. Biofilm disruption for the standard 8 strains are assessed and analyzed as previously described. In FIG. 35A-D, Applicants show that DNABII proteins synergize with polyamines to induce DNase resistance.

TABLE 1
Nucleases that prevent biofilm formation and their spermidine sensitivities

| Enzyme | Xhol | DNase* | Pstl | Sapl | T4 Pol | NucB | Bal31 |
|---|---|---|---|---|---|---|---|
| Spd | 50 μM | 300 μM | 500 μM | 10 mM | 10 mM | >10 mM | >10 mM |
| inhibition^{#} | | | | | | | |

*Pulmozyme;
^{#}Spermidine was titrated from 10 μM-10 mM to determine the concentration where each nuclease was reduced to <10% activity on genomic DNA.

### Testing the Robustness of the Proportions of Steady State Levels of DNA, DNABII, and Polyamines in Bacterial Biofilms.

While Applicants have shown that eDNA, DNABII proteins, and polyamines are each essential to maintain the TEDS of biofilms, the proportions of each component that interacts to productively maximize biofilm formation can be determined. When bacteria form biofilms, an equilibrium of planktonic/biofilm bacteria is achieved. Indeed, Applicants have previously shown that DNABII proteins are limiting for UPEC biofilms (Devaraj et al. (2015) Mol Microbiol. 96(6):1119-35), and that increasing concentrations of DNABII proteins drive planktonic bacteria into the biofilm state in a dose dependent manner. Devaraj et al. (2015) Mol Microbiol. 96(6):1119-35. UPEC that is deficient in HU produces a smaller biofilm but is more responsive to exogenous DNABII in driving planktonic bacteria into the biofilm state. Devaraj et al. (2015) Mol Microbiol. 96(6):1119-35. At the same time, UPEC biofilms that are depleted of eDNA (with DNase) no longer increase biofilm biomass upon exogenous DNABII addition. In contrast, any attempt to add exogenous DNABII proteins or DNA negatively impacts NTHI biofilms (data not shown). Applicants hypothesize that when biofilm EPS is deficient in DNA, DNABII protein, and polyamines, such that these 3 components are sub optimally present with respect to one another, the bacteria are preferentially partitioned into the planktonic state. Likewise, similar to UPEC, supplementation of the limiting component(s) to its optimal proportion should improve partitioning of bacteria into the biofilm state. In contrast, when all three components are in proper proportions, as Applicants believe in NTHI, supplementation of any of these components can disrupt the proper ratio and have a negative impact on biofilm formation. Here exogenous DNABII protein to native UPEC biofilms is titrated and the proportion of biofilm and planktonic bacteria is measured. Once biofilms are saturated with exogenous DNABII proteins, both polyamines and chromosomal DNA are varied to further partition bacteria into the biofilm state.

Each of the predominant polyamines (or combinations thereof) identified are tested and by varying concentrations up to 10-fold lesser and greater from the measured concentrations in the extracellular environment when biofilms are grown in CDM. Applicants hypothesize that if DNA, polyamines, and DNABII proteins are added in their proper proportions, it would drive planktonic bacteria into biofilms. Hence, WT UPEC and NTHI and their isogenic HU deficient strains (despite the remaining presence of IHF, the HU deficient strains still have a biofilm deficient phenotype) are also tested for the optimal proportion of HU, DNA, and polyamines. The effects of combinatorial biofilm component additions via the in vitro biofilm formation assay are evaluated through determination of the relative proportion of bacteria present in the planktonic phase vs the biofilm phase by dilution plating. Devaraj et al. (2015) Mol Microbiol. 96(6):1119-35. Applicants show in FIG. 42A-D that P11 addition prevents biofilm formation in a dose-dependent manner. Exogenous spermidine and HU together restore biofilm development but not either alone, which suggests that P11 anti-biofilm activity is the result of titration of structural components (polyamines, DNABII proteins) away from the biofilm EPS without direct contact.

Determine if there are Preferred Polyamines in the TEDS

As shown above, biofilms in a physically separated chamber can be disrupted by the cation-exchanger P11. Importantly, both polyamines and DNABII proteins have to be added back to the biofilm chamber to counteract this disruption, while each component by itself is ineffective. As spermine and spermidine modulate DNA structure more effectively than putrescine or cadaverine (Kabir et al. (2013) PLoS One. 8(7):e70510), polyamines singly and in combination are tested at various concentrations as described above to determine if specific polyamines are required to facilitate TEDS production. HU is added at defined concentrations (as described above as well as 5-fold lower and higher) throughout this experiment. This experiment is performed with the standard 8 strains as described above, quantifying the effects of the various polyamines on biofilm formation using the in vitro biofilm assay described in above.

### Outcomes, and Alternatives

A genetic approach can be applied to study TEDS formation by examining the contribution of deficiencies in UPEC polyamine synthesis and export genes on the steady state levels of extracellular polyamines. Single and combinations of mutations that yield deficient biofilm phenotypes are identified and are complemented with exogenous polyamines. The choice of UPEC as a model system is based on, its clinical importance (Subashchandrabose et al. (2015) Microbiol Spectr. 3(4)), the published characterization of the eDNA-DNABII dependent EPS (Devaraj et al. (2015) Mol Microbiol. 96(6):1119-35), and the extensive published understanding of the E. coli polyamine pathways (Tabor et al. (1985) Microbiol Rev. 49(1):81-99), albeit without examination of a biofilm phenotype (less comprehensive genetic analyses of biofilm phenotypes in other bacteria largely focused on intracellular roles of polyamines). Di Martino et al. (2013) Int J Med Microbiol. 303(8):484-91; Karatan et al. (2013) Biotechnol Lett. 35(11):1715-7. Which polyamines are present and in what combinations during biofilm development as well as clinically important, biofilm-forming pathogens are determined. Polyamines that naturally contribute to biofilm development are revealed. Whether constituent polyamines are simply binding to, or are binding to and altering, eDNA structure using nucleases that prevent biofilm formation but are inhibited to varying degrees by polyamines is determined. If the polyamines are not affecting DNA structure, then there would be a hierarchy of biofilm disruption related to the sensitivities of these nucleases (Table 1) to polyamines as polyamines accumulate with time. If a new nuclease-resistant DNA structure is formed, there would be an age of biofilm maturity where no nuclease functions despite permissive concentrations of polyamines. The expression of biosynthesis genes by qRT-PCR and of steady state levels of polyamines and DNABII proteins by Western analysis of untreated biofilms and those that have the strongest biofilm phenotype (greatest amount of bacteria partitioned into the biofilm state) is examined. Lastly, Endogenous polyamines is replaced with specific combinations of polyamines to test the robustness of polyamines in the TEDS to determine whether all polyamines equally efficacious in biofilm development.

Determine the Role of Z-DNA in the Development of the Tripartite eDNA-Dependent Scaffold.

As biofilms mature, eDNA within the biofilm EPS becomes DNase-resistant. Z-DNA is known to be DNase resistant and accumulates in the TEDS as biofilms mature.

Other pathogens singly, in defined mixed biofilms, and in human samples are examined to determine to what degree Z-DNA is universal amongst bacterial biofilms.

Reveal the Abundance of Z-DNA in Single Species Biofilms and Determine if Z-DNA Co-Localizes with Polyamines and/or DNABII Proteins

11 biofilm forming pathogens are examined {a standard 8 and 3 more [M. catarrhalis (common co-pathogen in OM), Porphyromonas gingivalis (periodontal pathogen), and Streptococcus gordonii (oral opportunistic pathogen)], that dual species biofilms below can be used and immunofluorescence using an anti-Z-DNA antibody as each biofilm ages (0, 4, 16, 24, 48, and 96 h) can be performed. Samples are probed with Z-DNA-specific antibodies and compared to a naïve antibody negative control and B-DNA specific antibody to rigorously identify Z-DNA presence. Parallel experiments are performed with IgG-enriched anti-polyamine and anti-DNABII antisera, and their respective naïve IgG controls, to determine if all three components co-localize within biofilm EPS. Biofilms are counterstained with a bacterial outer membrane stain such as FM4-64, antibody labeling detected with highly cross-adsorbed secondary antibodies from a common source, and imaged by CLSM. The degree of colocalization is quantified using the Coloc 2 analysis plugin in ImageJ. To examine whether the prevalence of Z-DNA correlates with partitioning of bacteria to the biofilm state, the ratio of planktonic/biofilm bacteria is quantified for each species as described herein.

Determine the Abundance of Z-DNA in Dual Species Biofilms and Determine if Z-DNA Co-Localizes with Polyamines and/or DNABII Proteins

Dual species biofilms are examined to determine how Z-DNA content, distribution, and interaction with polyamines and DNABII proteins changes with the age of the biofilm in the context of a polymicrobial environment. Polymicrobial combinations known to occur in disease and with which Applicants have experience in vitro include NTHI+M. catarrhalis (representative of polymicrobial OM biofilms), P. gingivalis+S. gordonii (representative of polymicrobial periodontal biofilms) 29, and NTHI+P. aeruginosa (representative of polymicrobial CF and chronic suppurative OM biofilms). Polymicrobial biofilm cultures are inoculated as directed by clinical observation; e.g. NTHI biofilm are established prior to P. aeruginosa inoculation, as P. aeruginosa is commonly a secondary invader at sites of NTHI infection. The resultant polymicrobial biofilms are analyzed for Z-DNA content as well as for polyamine and DNABII protein presence in the biofilm EPS by immunofluorescence as described previously.

Establish the Extent of Z-DNA in Dual Species Biofilms and if Z-DNA Colocalizes with Polyamines and/or DNABII Proteins when One Partner is Unable to Contribute eDNA, Polyamines, and HU.

HU deficient NTHI strain forms a mat-like biofilm that is devoid of polyamines and eDNA. This strain paired with common co-infecting pathogens, P. aeruginosa and M. catarrhalis, as well as WT NTHI (carrying a gfp expressing gene to distinguish it from the HU mutant) are used to determine if the eDNA, HU, and polyamines of each of the WT bacteria can complement the deficiency in the NTHI mutant which includes the formation of Z-DNA. Biofilm formation is quantified by CLSM as above, and EPS components and structure is analyzed by immunofluorescence CLSM. In FIG. 46A, immunofluorescence CLSM images of indicated biofilms probed with naive or anti-HU (gray) and anti-spermidine (Spd, light gray) antibodies are shown, the co-localization is indicated in white.

### Detection of Z-DNA in Human Samples.

As Applicants have previously done for DNABII proteins and polyamines (Gustave et al. (2013) J Cyst Fibros. 12(4):384-9; Idicula et al. (2016) Laryngoscope. 126(8):1946-51), Clinical samples from human biofilm-associated diseases are examined, including effusions recovered from children with OM (Idicula et al. (2016) Laryngoscope. 126(8):1946-51), CF sputum (Gustave et al. (2013) J Cyst Fibros. 12(4):384-9), and aspirates from adults with CRS, and determine by immunofluorescence microscopy if Z-DNA is present. Applicants have access to mixed-sex clinical samples from each of the aforementioned, for which Applicants have corresponding microbiological data. Sections of each (testing an equal number of male and female sourced clinical samples) is probed with Z-DNA-specific antibodies at varying concentrations while comparing immunofluorescence to a naïve antibody control. How Z-DNA detection relates spatially to polyamines and DNABII proteins by immunofluorescence as described previously is also investigated. In FIG. 46A, Applicants show immunofluorescence CLSM images of biofilms formed for 40 h of the indicated bacteria, probed with naive or anti-B-DNA (dark gray) and anti-Z-DNA antibodies (white). A well-characterized, Z-DNA-specific monoclonal antibody (clone Z2258,59,78) was used to detect Z-DNA. DNABII, polyamines, Z-DNA and B-DNA components are all present in the EPS of multiple bacterial biofilms. Z-DNA is an integral part of the EPS of multiple bacterial biofilms at different steady state levels.

### Investigate the Function of Z-DNA in the Development of the TEDS.

Here Applicants determine to what degree Z-DNA plays an integral structural role in the TEDS.

### Probe the TEDS Using Nucleases Specific for Z-DNA.

Based on the literature and Applicants' data, it is likely that while Z-DNA accumulates within an aging biofilm, substantial B form DNA remains (data not shown), meaning B-Z interfaces are present throughout the biofilm. Rich and coworkers created a Z-DNA specific nuclease by fusing the N-terminal Zα Z-DNA binding domain of the human Z-DNA binding protein ADAR1 to the C-terminal catalytic FN domain of the Type II restriction endonuclease Fokl (Kim et al. (1997) Proc Natl Acad Sci USA. 94(24):12875-9), where the Z-DNA binding domain positions the B-specific nuclease to the B-Z junctions. An improved version of this enzyme is constructed that displays higher Z-DNA specificity through dual hADARl Za domain (residues 133-209) fusion (Zaa) and produce, purify to >95% purity, and confirm Z-DNA cleavage activity by digestion of a Z-DNA insert in a supercoiled plasmid (as described in Shin et al. (2016) DNA Res.). As a complementary approach, 51 nuclease, an enzyme that displays hyperactivity at B-Z junctions is used. Kim et al. (1996) J Biol Chem. 271(16):9340-6. If Z-DNA is important to maintain the structural integrity of the TEDS, the use of these nucleases should disrupt the biofilm. Since these enzymes require Z-DNA for function, they can be a probe for Z-DNA throughout the course of biofilm development and serve as parallel means to measure the presence of Z-DNA (in addition to immunofluorescence). The ability of the aforementioned B-Z junction specific nucleases to inhibit biofilm formation through the course of development (0, 8, 16, 24, 48, and 96 h) is examined, using the in vitro biofilm assay as described previously. Whether degradation of Z-DNA tracts results in altered partitioning of biofilm and planktonic bacteria is determined above. The anti-biofilm activity of these nucleases against the standard 8 strains, as well as any additional species for which Applicants observe a relative prevalence of Z-DNA detection, is evaluated.

Determine if Driving DNA into the B Form Affects the TEDS

The equilibrium between B- and Z-DNA can be further driven into the B form with the addition of intercalating agents (e.g. ethidium bromide, chloroquine, DAPI (Kwakye-Berko et al. (1990) Mol Biochem Parasitol. 39(2):275-8; Shafer et al. (1984) Nucleic Acids Res. 12(11):4679-90; Kim et al. (1993) Biopolymers. 33(11):1677-86) or DNA binding molecules (e.g. netropsin, branched polyamines). Muramatsu et al. (2016) J Chem Phys. 145(23):235103; Zimmer et al. (1983) FEBS Lett. 154(1):156-60. If Z-DNA is the basis for the structural integrity of the TEDS then these agents should disrupt biofilms. To test this hypothesis, Applicants will use the in vitro biofilm assay to assess whether adding Z-to-B-DNA catalysts impede biofilm formation, assessing biofilm inhibition by CLSM, shifts in planktonic/biofilm partitioning by dilution plating, and changes in Z-DNA content by immunofluorescence microscopy. Minimal inhibitory concentration (MIC) for each compound can be determined by microplate dilution, then the dose-dependence of biofilm disruption and Z-DNA reversion at sub-MIC concentrations can be determined. These molecules are tested against the 8 standard strains, and the 3 molecules with the best antibiofilm activity are investigated further with any additional species for which Applicants observe a relative prevalence of Z-DNA detection.

Determine if Driving DNA into the Z Form Affects the TEDS.

The equilibrium between B- and Z-DNA can be driven towards Z-DNA by increasing the level of polycations (e.g. polyamines) (Jovin et al. (1987) Ann Rev Phys Chem. 38:521-60), by adding exogenous recombinant Zα domain derived from a member of the Zα domain family of proteins (ADAR proteins, ZBP proteins, poxvirus E3 proteins) that drive Z-DNA prone sequences into Z-DNA (Athanasiadis et al. (2012) Semin Cell Dev Biol. 23(3):275-80), or by methylating the C5 of cytosines in alternating purine pyrimidine tracts (e.g. HhaI, M.SssI, and M.CviPI methyltransferases)90. While the extent of biofilm formation may not change, the kinetics of biofilm formation and the overall structure likely will. Recombinant Zaa domain from hADARl, as described above, only without the Fokl nuclease fusion, is produced and Z-DNA binding activity of the recombinant protein is confirmed by CD. Zaa domain (0, 0.5, 5, or 50 μM) or methyltransferase (e.g. HhaI at 0, 0.1, 1, or 10 U/mL and S-adenosylmethionine) is added at biofilm seeding and biofilm formation at 4, 8, 16, 24, 48, and 96 h after seeding on the standard 8 strains and the otherwise isogenic HU deficient NTHI (a negative control that does not contain significant eDNA in its biofilm) is assessed with the in vitro biofilm assay using CLSM, shifts in planktonic/biofilm partitioning via dilution plating, and Z-DNA accumulation observed by immunofluorescence.

Determine ability of DNABII to bind Z-DNA specifically. While Applicants have shown that HU drives DNA into Z-DNA, additional analyses are performed to determine if DNABII proteins, polyamines, and DNA act synergistically to form Z-DNA are performed.

### Determine if IHF and HU Bind to DNA in the Presence of Polyamine-Induced Z-DNA

Extensive studies by Hud and co-workers (Sarkar et al. (2009) Biochemistry. 48(4):667-75; Sarkar et al. (2007) Nucleic Acids Res. 35(3):951-61) have shown that both IHF and HU bind and change the structure of DNA into thick fibers in the presence of spermidine. Whether defined DNA substrates [Holliday junction DNA, 35 bp duplexes that contain the IHF consensus sequence (WATCAANNNNTTR where W is A or T, N is any nucleotide and R is a purine), a scrambled version of the same sequence, 35 bp (dGdC) that is prone to Z-DNA conversion and 35 bp (dAdT) that is not] in the presence or absence of polyamines (0, 0.1, 0.5, 1, and 5 mM of the following: putrescine, spermidine, spermine, cadaverine, or combinations determined above) will form Z-DNA is determined. Z-DNA conversion via ellipticity measurement by CD spectroscopy anticipating the characteristic inversion at 250 and 280 nm is first determined. The binding of DNABII proteins to each substrate as judged by southwestern analysis (EMSA followed by immunoblot analysis with anti-Z-DNA and anti-DNABII antibodies) is then investigated. Goodman et al. (1989) Nature. 341(6239):251-4. Substrates (0.2 nM) that have CD confirmed Z-DNA conversion is incubated with 25-500 nM DNABII protein in 50 mM HEPES buffer pH 7.0 for 30 min at room temperature. The reaction mixtures are resolved by electrophoresis on a 6% non-denaturing polyacrylamide gel. ImageQuant software is used to quantify the band intensities. Equilibrium dissociation constants (K_{d}) is measured. Hung, et al. (2011) J Bacteriol. 193(14):3642-52. Because the DNA substrates is isotopically labeled, cell free conditioned medium from biofilms as a source of polyamines and/or DNABII proteins (0, 0.05, 0.1, 0.2 and 0.5 v/v of the subsequent reaction mixtures) from the same bacterial strains noted above can also be tested.

### Determine if IHF and HU Bind to Z-DNA

Whether IHF and HU bind to Z-DNA directly is determined. An EMSA with the DNABII proteins and a 35 bp brominated (dGdC) DNA substrate that is a stable Z-DNA conformer is performed. Herbert et al. (1993) Nucleic Acids Res. 21(11):2669-72. This experiment extends earlier findings since the brominated DNA substrate does not require polyamines to stabilize the Z-DNA state. Substrate DNA is labeled and prepared by incubation with α³²P-dGTP, 5-bromo dCTP, dGTP, and Klenow as described. Herbert et al. (1993) Nucleic Acids Res. 21(11):2669-72. 25-500 nM DNABII protein is incubated with 0.2 nM labeled DNA substrate, and the reaction mixtures is resolved by electrophoresis on a 6% non-denaturing polyacrylamide gel, band intensities quantified by ImageQuant software, and equilibrium dissociation constants (K_{d}) measured.

### Determine the Effectiveness of Agents Targeting EPS Components and Structures in Bacterial Biofilm Disruption

While eDNA and the DNABII are known targets for biofilm disruption, here Applicants focus on both polyamines and Z-DNA in the presence or absence of conventional antimicrobials. Applicants hypothesize that agents directed against the TEDS components and structures will act synergistically with conventional antimicrobials.

Determine Whether Agents Targeting Previously Discovered Components of the EPS eDNA Scaffold (Anti-DNABII Antibodies and Nucleases) are Synergistic with Agents that Target Newly Discovered TEDS Components/Structures (i.e. Polyamines and Z-DNA)

All three components or the structure of the TEDS as an approach for biofilm disruption, alone or in combination are targeted. Applicants hypothesize that by treating multiple targets within the TEDS Applicants can demonstrate synergism and thus greater potential for superior efficacy than targeting one target alone.

Targeting Polyamines, DNABII Proteins, and/or B-Form eDNA

Using known bacterial polyamine synthesis inhibitors [dicyclohexylamine (DCHA) (Mattila et al. (1984) Biochem J. 223(3):823-30), difluoromethylornithine (DFMO) (Muth et al. (2014) J Med Chem. 57(2):348-63), methylglyoxal-bis(cyclopentylamidino hydrazone) (MGBCP)(Takaji et al. (1997) Lett Appl Microbiol. 25(3):177-80)], the ability of each to disrupt biofilms formed by the standard 8 strains at distinct ages of maturation (0, 8, 24, and 48 h) is tested. The MIC for DCHA, DFMO, and MGBCP is determined for each pathogen, and a 100-fold dilution series below the MIC is tested for disruption activity. Each inhibitor is tested individually and in combination with DNase (Pulmozyme®; 0, 0.1, 1 U/ml) or IgG-enriched antiserum directed against HU (0, 150, 300 μg/ml). Biofilms are analyzed by LIVE/DEAD® staining and CLSM and biofilm parameters quantified previously. The effectiveness of each treatment combination compared to individual treatments and a no treatment control is assessed. In addition, the partition of CFUs that remain in the biofilm and planktonic to determine if there was any killing of the resident bacteria or simply disruption of the biofilm is examined. The 5 most effective combinations for all 8 pathogens on CF (as an example of a human polymicrobial biofilm-like community (Gustave et al. (2013) J Cyst Fibros. 12(4):384-9)) samples from 10 patients for the ability to disrupt the recalcitrant sputum biomass is then tested. Gustave et al. (2013) J Cyst Fibros. 12(4):384-9. Briefly, sputum samples are treated for 24 h and imaged. Disruption of the sputum samples as judged by increases in turbidity compared to untreated controls are used to quantify treatment effectiveness as previously described. Gustave et al. (2013) J Cyst Fibros. 12(4):384-9.

Targeting Z-DNA, DNABII Proteins, and/or B-Form eDNA.

Using the best Z-DNA inhibitors/disruptors (or best combinations thereof) as determined, the ability of each at sub-MIC concentrations, individually and in combination with anti-DNABII antibodies or DNase, to disrupt biofilms formed by the standard 8 strains at different ages of maturation is tested. If the Z-DNA specific nucleases utilized above inhibit biofilm formation, these nucleases are co-administered with anti-DNABII antibodies or DNase. Biofilm disruption activity and the most effective combinations for CF sputum disruption activity is analyzed.

Targeting polyamines, Z-DNA, DNABH proteins, and B-form eDNA

Using polyamine synthesis inhibitors and Z-DNA inhibitors, the ability of each in combination to disrupt biofilms formed by each of the standard 8 strains at different ages of maturation (0, 8, 24, and 48 h) is tested. The best dual treatment of polyamine inhibitor and Z-DNA inhibitor with anti-DNABII antibodies or DNase is also tested. Biofilm disruption for each combination of treatments is assessed. The top three combinations is tested against CF sputum.

### Determine Synergism of Treatments Directed Against the TEDS and Antimicrobials

The effectiveness of targeting all 3 components or structure of the TEDS for biofilm disruption when in combination with antimicrobials is examined.

Synergism of Targeting the TEDS with Antimicrobials.

Using the most effective biofilm disruption combinations, whether both the remaining resident biofilm bacteria and their respective planktonic bacteria can be killed by the co-administration of antimicrobials is determined. Using various concentrations and combinations for anti-DNABII antibodies, DNase, polyamine inhibitor, and Z-DNA converter determined above, Addition of appropriate antibiotics for biofilm disruption and killing against the standard 8 strains is tested. Clinically indicated antibiotics for each pathogen at 10-fold above and below the MIC are co-administered (E. faecium-0.1, 1, 10 μg/ml ampicillin (Weinstein et al. (2001) J Clin Microbiol. 39(7):2729-31); S. aureus-0.025, 0.25, 2.5 μg/ml clindamycin (LaPlante et al. (2008) Antimicrob Agents Chemother. 52(6):2156-62); S. epidermidis-0.2, 2, 20 mg/ml vancomycin (Pinheiro et al. (2016) Microb Drug Resist. 22(4):283-93); K. pneumoniae-0.4, 4, 40 μg/ml ceftazidime/avibactam (Sader, et al. (2017) Antimicrob Agents Chemother. 61(9)); A. baumannii-0.3, 3, 30 μg/ml meropenem (Liang et al. (2011) J Microbiol Immunol Infect. 44(5):358-63); P. aeruginosa-0.4, 4, 40 μg/ml colistin (Hindler et al. (2013) J Clin Microbiol. 51(6):1678-84); Enterobacter-0.4, 4, 40 μg/ml cefepime (Rivera et al. (2016) Antimicrob Agents Chemother. 60(6):3854-5), NTHI-0.1/0.05, 1/0.5, 10 μg/ml/5 mg/ml amoxicillin/clavulanatelithium31). Biofilm disruption and remaining viable partitioning for each combination of treatments is assessed and the combinations is tested against CF sputum.

With this better understanding of the universal TEDS, Applicants envision the potential to treat biofilms as a stand alone approach or combine this approach with strategies that target species-specific biofilm components (e.g. P. aeruginosa polysaccharides alginate, Pel, and Psl (Gunn et al. (2016) J Biol Chem. 291(24):12538-46)) to optimize the surgical effectiveness of biofilm disruption against specific pathogens.

Applicants also provide a pre-clinical model for tuberculosis (TB). See Ordway et al. (2010) Anti. Agents and Chemotherapy 54:1820. The microorganism Mycobacterium tuberculosis is responsible for a growing global epidemic. Current figures suggest that there are approximately 8 million new cases of TB and about 2.7 million deaths due to TB annually. In addition to the role of this microbe as a co-infection of individuals with HIV (of the ^{˜}45 million infected with HIV, estimates are that ^{˜}⅓ are also co-infected with M. tuberculosis), its particularly troublesome that isolates have become highly resistant to multiple drugs and no new drug for TB has been introduced in over a quarter of a century. In this animal model, SPF guinea pigs are maintained in a barrier colony and infected via aerosolized spray to deliver ^{˜}20 cfu of M. tuberculosis strain Erdman K01 bacilli into their lungs. Animals are sacrificed with determination of bacterial load and recovery of tissues for histopathological assessment on days 25, 50, 75, 100, 125 and 150 days post-challenge. Unlike mice which do not develop classic signs of TB, guinea pigs challenged in this manner develop well-organized granulomas with central necrosis, a hallmark of human disease. Further, like humans, guinea pigs develop severe pyogranulomatous and necrotizing lymphadenitis of the draining lymph nodes as part of the primary lesion complex. Use of this model provides a pre-clinical screen to confirm and identify therapeutic as well as preventative strategies for reduction and/or elimination of the resulting M. tuberculosis biofilms which have been observed to form in the lungs of these animals subsequent to challenge and are believed to contribute to both the pathogenesis and chronicity of the disease.

### Experiment No. 6

Multiple animal models of catheter/indwelling device biofilm infections are known. See Otto (2009) Nature Reviews Microbiology 7:555. While typically considered normal skin flora, the microbe Staphylococcus epidermidis has become what many regard as a key opportunistic pathogen, ranking first among causative agents of nosocomial infections. Primarily, this bacterium is responsible for the majority of infections that develop on indwelling medical devices which are contaminated by this common skin colonizer during device insertion. While not typically life-threatening, the difficulty associated with treatment of these biofilm infections, combined with their frequency, makes them a serious public health burden. Current costs associated with treatment of vascular catheter associated bloodstream infections alone that are due to S. epidermidis amount to $2 billion annually in the United States. In addition to S. epidermidis, E. faecalis and S. aureus are also contaminations found on indwelling medical devices. There are several animal models of catheter-associated S. epidermidis infections including rabbits, mice, guinea pigs and rats all of which are used to study the molecular mechanisms of pathogenesis and which lend themselves to studies of prevention and/or therapeutics. Rat jugular vein catheters have been used to evaluate therapies that interfere with E. faecalis, S. aureus and S. epidermidis biofilm formation. Biofilm reduction is often measured three ways—(i) sonicate catheter and calculate CFUs, (ii) cut slices of catheter or simply lay on a plate and score, or (iii) the biofilm can be stained with crystal violet or another dye, eluted, and OD measured as a proxy for CFUs.

### Experiment No. 7

Methods described herein may be used to confer passive immunity on a non-immune subject. Passive immunity against a given antigen may be conferred through the transfer of antibodies or antigen binding fragments that specifically recognize or bind to a particular antigen. Antibody donors and recipients may be human or non-human subjects. Additionally, or alternatively, the antibody composition may comprise an isolated or recombinant polynucleotide encoding an antibody or antigen binding fragment that specifically recognizes or binds to a particular antigen.

Passive immunity may be conferred in cases where the administration of immunogenic compositions poses a risk for the recipient subject, the recipient subject is immuno-compromised, or the recipient subject requires immediate immunity. Immunogenic compositions may be prepared in a manner consistent with the selected mode of administration. Compositions may comprise whole antibodies, antigen binding fragments, polyclonal antibodies, monoclonal antibodies, antibodies generated in vivo, antibodies generated in vitro, purified or partially purified antibodies, or whole serum. Administration may comprise a single dose of an antibody composition, or an initial administration followed by one or more booster doses. Booster doses may be provided a day, two days, three days, a week, two weeks, three weeks, one, two, three, six or twelve months, or at any other time point after an initial dose. A booster dose may be administered after an evaluation of the subject's antibody titer.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Dillon S C, Dorman C J. Bacterial nucleoid-associated proteins, nucleoid structure and gene expression. Nat Rev Microbiol. 2010; 8(3):185-95. doi: 10.1038/nrmicro2261. PubMed PMID: 20140026.
2. Dey D, Nagaraja V, Ramakumar S. Structural and evolutionary analyses reveal determinants of DNA binding specificities of nucleoid-associated proteins HU and IHF. Mol Phylogenet Evol. 2017; 107:356-66. doi: 10.1016/j.ympev.2016.11.014. PubMed PMID: 27894997.
3. Swinger K K, Rice P A. IHF and HU: flexible architects of bent DNA. Curr Opin Struct Biol. 2004; 14(1):28-35. doi: 10.1016/j.sbi.2003.12.003. PubMed PMID: 15102446.
4. Avery O T, Macleod C M, McCarty M. Studies on the Chemical Nature of the Substance Inducing Transformation of Pneumococcal Types: Induction of Transformation by a Desoxyribonucleic Acid Fraction Isolated from Pneumococcus Type Iii. J Exp Med. 1944; 79(2):137-58. PubMed PMID: 19871359; PMCID: PMC2135445.
5. Gunn J S, Bakaletz L O, Wozniak D J. What's on the Outside Matters: The Role of the Extracellular Polymeric Substance of Gram-negative Biofilms in Evading Host Immunity and as a Target for Therapeutic Intervention. J Biol Chem. 2016; 291(24):12538-46. doi: 10.1074/jbc.R115.707547. PubMed PMID: 27129225; PMCID: PMC4933457.
6. Dongari-Bagtzoglou A. Pathogenesis of mucosal biofilm infections: challenges and progress. Expert Rev Anti Infect Ther. 2008; 6(2):201-8. doi: 10.1586/14787210.6.2.201. PubMed PMID: 18380602; PMCID: PMC2712878.
7. Steele L, Fawal H. 4th Decennial International Conference on nosocomial and healthcare-associated infections: a challenge for change. Am J Infect Control. 2000; 28(3):207-10. doi: 10.1067/mic.2000.107275. PubMed PMID: 10840339.
8. Apicella M A. Bacterial otitis media, the chinchilla middle ear, and biofilms. J Infect Dis. 2009; 199(6):774-5. PubMed PMID: 19434910.
9. Foxman B, Barlow R, D'Arcy H, Gillespie B, Sobel J D. Urinary tract infection: self-reported incidence and associated costs. Ann Epidemiol. 2000; 10(8):509-15. PubMed PMID: 11118930.
10. Mancl K A, Kirsner R S, Ajdic D. Wound biofilms: lessons learned from oral biofilms. Wound Repair Regen. 2013; 21(3):352-62. doi: 10.1111/wrr.12034. PubMed PMID: 23551419; PMCID: PMC3648594.
11. Pendleton J N, Gorman S P, Gilmore B F. Clinical relevance of the ESKAPE pathogens. Expert Rev Anti Infect Ther. 2013; 11(3):297-308. doi: 10.1586/eri.13.12. PubMed PMID: 23458769.
12. Ceri H, Olson M E, Stremick C, Read R R, Morck D, Buret A. The Calgary Biofilm Device: new technology for rapid determination of antibiotic susceptibilities of bacterial biofilms. J Clin Microbiol. 1999; 37(6):1771-6. PubMed PMID: 10325322; PMCID: PMC84946.
13. Stacy A, McNally L, Darch S E, Brown S P, Whiteley M. The biogeography of polymicrobial infection. Nat Rev Microbiol. 2016; 14(2):93-105. doi: 10.1038/nrmicro.2015.8. PubMed PMID: 26714431; PMCID: PMC5116812.
14. Wolcott R, Costerton J W, Raoult D, Cutler S J. The polymicrobial nature of biofilm infection. Clin Microbiol Infect. 2013; 19(2):107-12. doi: 10.1111/j.1469-0691.2012.04001.x. PubMed PMID: 22925473.
15. Jurcisek J A, Brockman K L, Novotny L A, Goodman S D, Bakaletz L O. Nontypeable Haemophilus influenzae releases DNA and DNABII proteins via a T4SS-like complex and ComE of the type IV pilus machinery. Proc Natl Acad Sci USA. 2017; 114(32):E6632-E41. doi: 10.1073/pnas.1705508114. PubMed PMID: 28696280; PMCID: PMC5559034.
16. Sena-Velez M, Redondo C, Graham J H, Cubero J. Presence of Extracellular DNA during Biofilm Formation by Xanthomonas citri subsp. citri Strains with Different Host Range. PLoS One. 2016; 11(6):e0156695. doi: 10.1371/journal.pone.0156695. PubMed PMID: 27248687; PMCID: PMC4889101.
17. Wang S, Liu X, Liu H, Zhang L, Guo Y, Yu S, Wozniak D J, Ma L Z. The exopolysaccharide Psl-eDNA interaction enables the formation of a biofilm skeleton in Pseudomonas aeruginosa. Environ Microbiol Rep. 2015; 7(2):330-40. doi: 10.1111/1758-2229.12252. PubMed PMID: 25472701; PMCID: PMC4656019.
18. Whitchurch C B, Tolker-Nielsen T, Ragas P C, Mattick J S. Extracellular DNA required for bacterial biofilm formation. Science. 2002; 295(5559):1487. doi: 10.1126/science.295.5559.1487. PubMed PMID: 11859186.
19. Frederiksen B, Pressler T, Hansen A, Koch C, Hoiby N. Effect of aerosolized rhDNase (Pulmozyme) on pulmonary colonization in patients with cystic fibrosis. Acta Paediatr. 2006; 95(9):1070-4. doi: 10.1080/08035250600752466. PubMed PMID: 16938752.
20. Martins M, Henriques M, Lopez-Ribot J L, Oliveira R. Addition of DNase improves the in vitro activity of antifungal drugs against Candida albicans biofilms. Mycoses. 2012; 55(1):80-5. doi: 10.1111/j.1439-0507.2011.02047.x. PubMed PMID: 21668524; PMCID: PMC3175262.
21. Hymes S R, Randis T M, Sun T Y, Ratner A J. DNase inhibits Gardnerella vaginalis biofilms in vitro and in vivo. J Infect Dis. 2013; 207(10):1491-7. doi: 10.1093/infdis/jit047. PubMed PMID: 23431033; PMCID: PMC3627197.
22. Goodman S D, Obergfell K P, Jurcisek J A, Novotny L A, Downey J S, Ayala E A, Tjokro N, Li B, Justice S S, Bakaletz L O. Biofilms can be dispersed by focusing the immune system on a common family of bacterial nucleoid-associated proteins. Mucosal Immunol. 2011; 4(6):625-37. doi: 10.1038/mi.2011.27. PubMed PMID: 21716265.
23. Hall-Stoodley L, Nistico L, Sambanthamoorthy K, Dice B, Nguyen D, Mershon W J, Johnson C, Hu F Z, Stoodley P, Ehrlich G D, Post J C. Characterization of biofilm matrix, degradation by DNase treatment and evidence of capsule downregulation in Streptococcus pneumoniae clinical isolates. BMC Microbiol. 2008; 8:173. doi: 10.1186/1471-2180-8-173. PubMed PMID: 18842140; PMCID: PMC2600794.
24. Izano E A, Shah S M, Kaplan J B. Intercellular adhesion and biocide resistance in nontypeable Haemophilus influenzae biofilms. Microb Pathog. 2009; 46(4):207-13. doi: 10.1016/j.micpath.2009.01.004. PubMed PMID: 19490830; PMCID: PMC2691864.
25. Kaplan J B, LoVetri K, Cardona S T, Madhyastha S, Sadovskaya I, Jabbouri S, Izano E A. Recombinant human DNase I decreases biofilm and increases antimicrobial susceptibility in staphylococci. J Antibiot (Tokyo). 2012; 65(2):73-7. doi: 10.1038/ja.2011.113. PubMed PMID: 22167157; PMCID: PMC3288126.
26. Novotny L A, Amer A O, Brockson M E, Goodman S D, Bakaletz L O. Structural stability of Burkholderia cenocepacia biofilms is reliant on eDNA structure and presence of a bacteriuml nucleic acid binding protein. PLoS One. 2013; 8(6):e67629. doi: 10.1371/journal.pone.0067629. PubMed PMID: 23799151; PMCID: PMC3682984.
27. Tetz V V, Tetz G V. Effect of extracellular DNA destruction by DNase I on characteristics of forming biofilms. DNA Cell Biol. 2010; 29(8):399-405. doi: 10.1089/dna.2009.1011. PubMed PMID: 20491577.
28. Devaraj A, Justice S S, Bakaletz L O, Goodman S D. DNABII proteins play a central role in UPEC biofilm structure. Mol Microbiol. 2015; 96(6):1119-35. doi: 10.1111/mmi.12994. PubMed PMID: 25757804; PMCID: PMC4464964.
29. Rocco C J, Davey M E, Bakaletz L O, Goodman S D. Natural antigenic differences in the functionally equivalent extracellular DNABII proteins of bacterial biofilms provide a means for targeted biofilm therapeutics. Mol Oral Microbiol. 2017; 32(2):118-30. doi: 10.1111/omi.12157. PubMed PMID: 26988714; PMCID: PMC5023461.
30. Devaraj A, Buzzo J, Rocco C J, Bakaletz L O, Goodman S D. The DNABII family of proteins is comprised of the only nucleoid associated proteins required for nontypeable Haemophilus influenzae biofilm structure. Microbiologyopen. 2017. doi: 10.1002/mbo3.563. PubMed PMID: 29230970.
31. Brockson M E, Novotny L A, Mokrzan E M, Malhotra S, Jurcisek J A, Akbar R, Devaraj A, Goodman S D, Bakaletz L O. Evaluation of the kinetics and mechanism of action of anti integration host factor-mediated disruption of bacterial biofilms. Mol Microbiol. 2014; 93(6):1246-58. doi: 10.1111/mmi.12735. PubMed PMID: 25069521; PMCID: PMC4160410.
32. Michael A J. Biosynthesis of polyamines and polyamine-containing molecules. Biochem J. 2016; 473(15):2315-29. doi: 10.1042/BCJ20160185. PubMed PMID: 27470594.
33. Tabor C W, Tabor H. Polyamines in microorganisms. Microbiol Rev. 1985; 49(1):81-99. PubMed PMID: 3157043; PMCID: PMC373019.
34. Bachrach U. Naturally occurring polyamines: interaction with macromolecules. Curr Protein Pept Sci. 2005; 6(6):559-66. PubMed PMID: 16381604.
35. Pasini A, Caldarera C M, Giordano E. Chromatin remodeling by polyamines and polyamine analogs. Amino Acids. 2014; 46(3):595-603. doi: 10.1007/s00726-013-1550-9. PubMed PMID: 23836422.
36. Sarkar T, Petrov A S, Vitko J R, Santai C T, Harvey S C, Mukerji I, Hud N V. Integration host factor (IHF) dictates the structure of polyamine-DNA condensates: implications for the role of IHF in the compaction of bacterial chromatin. Biochemistry. 2009; 48(4):667-75. doi: 10.1021/bi8019965. PubMed PMID: 19132923.
37. Sarkar T, Vitoc I, Mukerji I, Hud N V. Bacterial protein HU dictates the morphology of DNA condensates produced by crowding agents and polyamines. Nucleic Acids Res. 2007; 35(3):951 61. doi: 10.1093/nar/gk11093. PubMed PMID: 17259223; PMCID: PMC1807954.
38. Wilton M, Charron-Mazenod L, Moore R, Lewenza S. Extracellular DNA Acidifies Biofilms and Induces Aminoglycoside Resistance in Pseudomonas aeruginosa. Antimicrob Agents Chemother. 2015; 60(1):544-53. doi: 10.1128/AAC.01650-15. PubMed PMID: 26552982; PMCID: PMC4704225.
39. Thomas T J, Messner R P. A left-handed (Z) conformation of poly(dA-dC).poly(dG-dT) induced by polyamines. Nucleic Acids Res. 1986; 14(16):6721-33. PubMed PMID: 3748821; PMCID: PMC311676.
40. Thomas T J, Messner R P. Structural specificity of polyamines in left-handed Z-DNA formation. Immunological and spectroscopic studies. J Mol Biol. 1988; 201(2):463-7. PubMed PMID: 3418706.
41. Pohl F M. Salt-induced transition between two double-helical forms of oligo (dC-dG). Cold Spring Harb Symp Quant Biol. 1983; 47 Pt 1:113-7. PubMed PMID: 6574834.
42. Pohl F M. Dynamics of the B-to-Z transition in supercoiled DNA. Proc Natl Acad Sci USA. 1986; 83(14):4983-7. PubMed PMID: 3460079; PMCID: PMC323873.
43. Rahmouni A R. Z-DNA as a probe for localized supercoiling in vivo. Mol Microbiol. 1992; 6(5):569-72. PubMed PMID: 1552856.
44. Jovin T M S, D. M.; McIntosh, L. P. The Transition Between B-DNA and Z-DNA. Ann Rev Phys Chem. 1987; 38:521-60.
45. Kim D H, Im H, Jee J G, Jang S B, Yoon H J, Kwon A R, Kang S M, Lee B J. beta-Arm flexibility of HU from Staphylococcus aureus dictates the DNA-binding and recognition mechanism. Acta Crystallogr D Biol Crystallogr. 2014; 70(Pt 12):3273-89. doi: 10.1107/S1399004714023931. PubMed PMID: 25478845.
46. Chaires J B, Norcum M T. Structure and stability of Z* DNA. J Biomol Struct Dyn. 1988; 5(6):1187-207.doi: 10.1080/07391102.1988. Ser. No. 10/506,463. PubMed PMID: 3271507.
47. Thomas T J, Bloomfield V A. Chain flexibility and hydrodynamics of the B and Z forms of poly(dGdC). poly(dG-dC). Nucleic Acids Res. 1983; 11(6):1919-30. PubMed PMID: 6835843; PMCID: PMC325846.
48. Athanasiadis A. Zalpha-domains: at the intersection between RNA editing and innate immunity. Semin Cell Dev Biol. 2012; 23(3):275-80. doi: 10.1016/j.semcdb.2011.11.001. PubMed PMID: 22085847.
49. Bergen H R, 3rd, Losman M J, O'Connor T, Zacharias W, Larson J E, Accavitti M A, Wells R D, Koopman W J. Specificity of monoclonal anti-Z-DNA antibodies from unimmunized MRL/Mp-lpr/lpr mice. J Immunol. 1987; 139(3):743-8. PubMed PMID: 3496387.
50. Herbert A, Rich A. The biology of left-handed Z-DNA. J Biol Chem. 1996; 271(20):11595-8. PubMed PMID: 8662853.
51. Gustave J E, Jurcisek J A, McCoy K S, Goodman S D, Bakaletz L O. Targeting bacterial integration host factor to disrupt biofilms associated with cystic fibrosis. J Cyst Fibros. 2013; 12(4):384-9. doi: 10.1016/j.jcf2012.10.011. PubMed PMID: 23168017; PMCID: PMC3582735.
52. Novotny L A, Jurcisek J A, Goodman S D, Bakaletz L O. Monoclonal antibodies against DNA-binding tips of DNABII proteins disrupt biofilms in vitro and induce bacterial clearance in vivo. EBioMedicine. 2016; 10:33-44. doi: 10.1016/j.ebiom.2016.06.022. PubMed PMID: 27342872; PMCID: PMC5006588.
53. Freire M O, Devaraj A, Young A, Navarro J B, Downey J S, Chen C, Bakaletz L O, Zadeh H H, Goodman S D. A bacteriuml-biofilm-induced oral osteolytic infection can be successfully treated by immuno-targeting an extracellular nucleoid-associated protein. Mol Oral Microbiol. 2017; 32(1):74-88. doi: 10.1111/omi.12155. PubMed PMID: 26931773; PMCID: PMC5010536.
54. Bakaletz L O. Chinchilla as a robust, reproducible and polymicrobial model of otitis media and its prevention. Expert Rev Vaccines. 2009; 8(8):1063-82. doi: 10.1586/erv.09.63. PubMed PMID: 19627188.
55. Iacomino G, Picariello G, Sbrana F, Di Luccia A, Raiteri R, D'Agostino L. DNA is wrapped by the nuclear aggregates of polyamines: the imaging evidence. Biomacromolecules. 2011; 12(4):1178-86. doi: 10.1021/bm101478j. PubMed PMID: 21401020.
56. Paulin L, Lindberg L A, Poso H. Reversible inhibition of flagella formation after specific inhibition of spermidine synthesis by dicyclohexylamine in Pseudomonas aeruginosa. Antonie Van Leeuwenhoek. 1986; 52(6):483-90. PubMed PMID: 3101591.
57. Pegg A E, Bitonti A J, McCann P P, Coward J K. Inhibition of bacterial aminopropyltransferases by Sadenosyl-1,8-diamino-3-thiooctane and by dicyclohexylamine. FEBS Lett. 1983; 155(2):192-6. PubMed PMID: 6406265.
58. Heydorn A, Nielsen A T, Hentzer M, Sternberg C, Givskov M, Ersboll B K, Molin S. Quantification of biofilm structures by the novel computer program COMSTAT. Microbiology. 2000; 146 (Pt 10):2395-407. doi: 10.1099/00221287-146-10-2395. PubMed PMID: 11021916.
59. Yang C L, Chilvers M, Montgomery M, Nolan S J. Dornase alfa for cystic fibrosis. Paediatr Respir Rev. 2017; 21:65-7. doi: 10.1016/j.prrv.2016.09.001. PubMed PMID: 27769785.
60. Jang Y J, Lee C, Kim S K. Formation of Poly[d(A-T)2] Specific Z-DNA by a Cationic Porphyrin. Sci Rep. 2015; 5:9943. doi: 10.1038/srep09943. PubMed PMID: 25943171; PMCID: PMC4421867.
61. Segall A M, Goodman S D, Nash H A. Architectural elements in nucleoprotein complexes: interchangeability of specific and non-specific DNA binding proteins. EMBO J. 1994; 13(19):4536-48. PubMed PMID: 7925295; PMCID: PMC395386.
62. Tjokro N O, Rocco C J, Priyadarshini R, Davey M E, Goodman S D. A biochemical analysis of the interaction of Porphyromonas gingivalis HU PG0121 protein with DNA. PLoS One. 2014; 9(3):e93266. doi: 10.1371/journal.pone.0093266. PubMed PMID: 24681691; PMCID: PMC3969353.
63. Ayala E, Downey J S, Mashburn-Warren L, Senadheera D B, Cvitkovitch D G, Goodman S D. A biochemical characterization of the DNA binding activity of the response regulator VicR from Streptococcus mutans. PLoS One. 2014; 9(9):e108027. doi: 10.1371/journal.pone.0108027. PubMed PMID: 25229632; PMCID: PMC4168254.
64. Hung D C, Downey J S, Ayala E A, Kreth J, Mair R, Senadheera D B, Qi F, Cvitkovitch D G, Shi W, Goodman S D. Characterization of DNA binding sites of the ComE response regulator from Streptococcus mutans. J Bacteriol. 2011; 193(14):3642-52. doi: 10.1128/JB.00155-11. PubMed PMID: 21602345; PMCID: PMC3133340.
65. Idicula W K, Jurcisek J A, Cass N D, Ali S, Goodman S D, Elmaraghy C A, Jatana K R, Bakaletz L O. Identification of biofilms in post-tympanostomy tube otorrhea. Laryngoscope. 2016; 126(8):1946-51. doi: 10.1002/lary.25826. PubMed PMID: 27426942; PMCID: PMC4955598.
66. Brandstetter K A, Jurcisek J A, Goodman S D, Bakaletz L O, Das S. Antibodies directed against integration host factor mediate biofilm clearance from Nasopore. Laryngoscope. 2013; 123(11):2626-32. doi: 10.1002/lary.24183. PubMed PMID: 23670606; PMCID: PMC4060527.
67. Subashchandrabose S, Mobley H L. Virulence and Fitness Determinants of Uropathogenic Escherichia coli. Microbiol Spectr. 2015; 3(4). doi: 10.1128/microbiolspec.UTI-0015-2012. PubMed PMID: 26350328; PMCID: PMC4566162.
68. Subashchandrabose S, Mobley H L. Virulence and Fitness Determinants of Uropathogenic Escherichia coli. Microbiol Spectr. 2015; 3(4). doi: 10.1128/microbiolspec.UTI-0015-2012. PubMed PMID: 26350328; PMCID: PMC4566162.
69. Justice S S, Li B, Downey J S, Dabdoub S M, Brockson M E, Probst G D, Ray W C, Goodman S D. Aberrant community architecture and attenuated persistence of uropathogenic Escherichia coli in the absence of individual IHF subunits. PLoS One. 2012; 7(10):e48349. doi: 10.1371/journal.pone.0048349. PubMed PMID: 23133584; PMCID: PMC3485042.
70. Baba T, Ara T, Hasegawa M, Takai Y, Okumura Y, Baba M, Datsenko K A, Tomita M, Wanner B L, Mori H. Construction of Escherichia coli K-12 in-frame, single-gene knockout mutants: the Keio collection. Mol Syst Biol. 2006; 2:2006 0008. doi: 10.1038/msb4100050. PubMed PMID: 16738554; PMCID: PMC1681482.
71. Karperien A. FracLac for ImageJ 1999-2013. Available from: rsb.info.nih.gov/ij/plugins/fraclac/FLHelp/Introduction.htm.
72. Kashiwagi K, Miyamoto S, Suzuki F, Kobayashi H, Igarashi K. Excretion of putrescine by the putrescine-ornithine antiporter encoded by the potE gene of Escherichia coli. Proc Natl Acad Sci USA. 1992; 89(10):4529-33. PubMed PMID: 1584788; PMCID: PMC49116.
73. Soksawatmaekhin W, Kuraishi A, Sakata K, Kashiwagi K, Igarashi K. Excretion and uptake of cadaverine by CadB and its physiological functions in Escherichia coli. Mol Microbiol. 2004; 51(5):1401-12. doi: 10.1046/j.1365-2958.2003.03913.x. PubMed PMID: 14982633.
74. Higashi K, Ishigure H, Demizu R, Uemura T, Nishino K, Yamaguchi A, Kashiwagi K, Igarashi K. Identification of a spermidine excretion protein complex (MdtJI) in Escherichia coli. J Bacteriol. 2008; 190(3):872-8. doi: 10.1128/JB.01505-07. PubMed PMID: 18039771; PMCID: PMC2223573.
75. Sugiyama Y, Nakamura A, Matsumoto M, Kanbe A, Sakanaka M, Higashi K, Igarashi K, Katayama T, Suzuki H, Kurihara S. A Novel Putrescine Exporter SapBCDF of Escherichia coli. J Biol Chem. 2016; 291(51):26343-51. doi: 10.1074/jbc.M116.762450. PubMed PMID: 27803167; PMCID: PMC5159496.
76. Sabate Bresco M, Harris L G, Thompson K, Stanic B, Morgenstern M, O'Mahony L, Richards R G, Moriarty T F. Pathogenic Mechanisms and Host Interactions in Staphylococcus epidermidis Device-Related Infection. Front Microbiol. 2017; 8:1401. doi: 10.3389/fmicb.2017.01401. PubMed PMID: 28824556; PMCID: PMC5539136.
77. Hakkinen M R, Roine A, Auriola S, Tuokko A, Veskimae E, Keinanen T A, Lehtimaki T, Oksala N, Vepsalainen J. Analysis of free, mono- and diacetylated polyamines from human urine by LC-MS/MS. J Chromatogr B Analyt Technol Biomed Life Sci. 2013; 941:81-9. doi: 10.1016/j.jchromb.2013.10.009. PubMed PMID: 24185098.
78. Liu R, Li Q, Ma R, Lin X, Xu H, Bi K. Determination of polyamine metabolome in plasma and urine by ultrahigh performance liquid chromatography-tandem mass spectrometry method: application to identify potential markers for human hepatic cancer. Anal Chim Acta. 2013; 791:36-45. doi: 10.1016/j.aca.2013.06.044. PubMed PMID: 23890604.
79. Xu H, Liu R, He B, Bi C W, Bi K, Li Q. Polyamine Metabolites Profiling for Characterization of Lung and Liver Cancer Using an LC-Tandem M S Method with Multiple Statistical Data Mining Strategies: Discovering Potential Cancer Biomarkers in Human Plasma and Urine. Molecules. 2016; 21(8). doi: 10.3390/molecules21081040. PubMed PMID: 27517900.
80. Kabir A, Suresh Kumar G. Binding of the biogenic polyamines to deoxyribonucleic acids of varying base composition: base specificity and the associated energetics of the interaction. PLoS One. 2013; 8(7):e70510. doi: 10.1371/journal.pone.0070510. PubMed PMID: 23894663; PMCID: PMC3722294.
81. Di Martino M L, Campilongo R, Casalino M, Micheli G, Colonna B, Prosseda G. Polyamines: emerging players in bacteria-host interactions. Int J Med Microbiol. 2013; 303(8):484-91. doi: 10.1016/j.ijmm.2013.06.008. PubMed PMID: 23871215.
82. Karatan E, Michael A J. A wider role for polyamines in biofilm formation. Biotechnol Lett. 2013; 35(11):1715-7. doi: 10.1007/s10529-013-1286-3. PubMed PMID: 23881324.
83. Kim Y G, Kim P S, Herbert A, Rich A. Construction of a Z-DNA-specific restriction endonuclease. Proc Natl Acad Sci USA. 1997; 94(24):12875-9. PubMed PMID: 9371768; PMCID: PMC24231.
84. Shin S I, Ham S, Park J, Seo S H, Lim C_{H}, Jeon H, Huh J, Roh T Y. Z-DNA-forming sites identified by ChIP-Seq are associated with actively transcribed regions in the human genome. DNA Res. 2016. doi: 10.1093/dnares/dsw031. PubMed PMID: 27374614; PMCID: PMC5066173.
85. Kim J, Yang C, DasSarma S. Analysis of left-handed Z-DNA formation in short d(CG)n sequences in Escherichia coli and Halobacterium halobium plasmids. Stabilization by increasing repeat length and DNA supercoiling but not salinity. J Biol Chem. 1996; 271(16):9340-6. PubMed PMID: 8621598.
86. Kwakye-Berko F, Meshnick S. Sequence preference of chloroquine binding to DNA and prevention of Z-DNA formation. Mol Biochem Parasitol. 1990; 39(2):275-8. PubMed PMID: 2320060.
87. Shafer R H, Brown S C, Delbarre A, Wade D. Binding of ethidium and bis(methidium)spermine to Z DNA by intercalation. Nucleic Acids Res. 1984; 12(11):4679-90. PubMed PMID: 6739293; PMCID: PMC318867.
88. Kim S K, Eriksson S, Norden B. Z->B transition in poly[d(G-m5C)2] induced by interaction with 4′,6-diamidino-2-phenylindole. Biopolymers. 1993; 33(11):1677-86. doi: 10.1002/bip.360331105. PubMed PMID: 8241427.
89. Muramatsu A, Shimizu Y, Yoshikawa Y, Fukuda W, Umezawa N, Horai Y, Higuchi T, Fujiwara S, Imanaka T, Yoshikawa K. Naturally occurring branched-chain polyamines induce a crosslinked meshwork structure in a giant DNA. J Chem Phys. 2016; 145(23):235103. doi: 10.1063/1.4972066. PubMed PMID: 28010109.
90. Zimmer C, Marck C, Guschlbauer W. Z-DNA and other non-B-DNA structures are reversed to B-DNA by interaction with netropsin. FEBS Lett. 1983; 154(1):156-60. PubMed PMID: 6299791.
91. Temiz N A, Donohue D E, Bacolla A, Luke B T, Collins J R. The role of methylation in the intrinsic dynamics of B- and Z-DNA. PLoS One. 2012; 7(4):e35558. doi: 10.1371/journal.pone.0035558. PubMed PMID: 22530050; PMCID: PMC3328458.
92. Goodman S D, Nash H A. Functional replacement of a protein-induced bend in a DNA recombination site. Nature. 1989; 341(6239):251-4. doi: 10.1038/341251a0. PubMed PMID: 2528697.
93. Herbert A G, Rich A. A method to identify and characterize Z-DNA binding proteins using a linear oligodeoxynucleotide. Nucleic Acids Res. 1993; 21(11):2669-72. PubMed PMID: 8332463; PMCID: PMC309597.
94. Shishido K, Sakaguchi R, Nosoh Y. Enhancement of Si nuclease-susceptibility of negatively superhelical DNA by netropsin. Biochem Biophys Res Commun. 1984; 124(2):388-92. PubMed PMID: 6093797.
95. Mattila T, Honkanen-Buzalski T, Poso H. Reversible inhibition of bacterial growth after specific inhibition of spermidine synthase by dicyclohexylamine. Biochem J. 1984; 223(3):823 30. PubMed PMID: 6508744; PMCID: PMC1144368.
96. Muth A, Madan M, Archer J J, Ocampo N, Rodriguez L, Phanstiel Ot. Polyamine transport inhibitors: design, synthesis, and combination therapies with difluoromethylornithine. J Med Chem. 2014; 57(2):348-63. doi: 10.1021/jm401174a. PubMed PMID: 24405276.
97. Takaji S, Hibasami H, Imoto I, Hashimoto Y, Nakao K, Ikemura N, Taguchi Y, Nakashima K. Growth inhibition of Helicobacter pylori by a polyamine synthesis inhibitor, methylglyoxal bis(cyclopentylamidinohydrazone). Lett Appl Microbiol. 1997; 25(3):177-80. PubMed PMID: 9351259.
98. Weinstein M P. Comparative evaluation of penicillin, ampicillin, and imipenem MICs and susceptibility breakpoints for vancomycin-susceptible and vancomycin-resistant Enterococcus faecalis and Enterococcus faecium. J Clin Microbiol. 2001; 39(7):2729-31. doi: 10.1128/JCM.39.7.2729-2731.2001. PubMed PMID: 11427608; PMCID: PMC88224.
99. LaPlante K L, Leonard S N, Andes D R, Craig W A, Rybak M J. Activities of clindamycin, daptomycin, doxycycline, linezolid, trimethoprim-sulfamethoxazole, and vancomycin against community-associated methicillin-resistant Staphylococcus aureus with inducible clindamycin resistance in murine thigh infection and in vitro pharmacodynamic models. Antimicrob Agents Chemother. 2008; 52(6):2156-62. doi: 10.1128/AAC.01046-07. PubMed PMID: 18411321; PMCID: PMC2415789.
100. Pinheiro L, Brito C I, Pereira V C, Oliveira A, Bartolomeu A R, Camargo C H, Cunha M L. Susceptibility Profile of Staphylococcus epidermidis and Staphylococcus haemolyticus Isolated from Blood Cultures to Vancomycin and Novel Antimicrobial Drugs over a Period of 12 Years. Microb Drug Resist. 2016; 22(4):283-93. doi: 10.1089/mdr.2015.0064. PubMed PMID: 26623676.
101. Sader H S, Mendes R E, Streit J M, Flamm R K. Antimicrobial Susceptibility Trends among Staphylococcus aureus Isolates from U.S. Hospitals: Results from 7 Years of the Ceftaroline (AWARE) Surveillance Program, 2010 to 2016. Antimicrob Agents Chemother. 2017; 61(9). doi: 10.1128/AAC.01043-17. PubMed PMID: 28630196; PMCID: PMC5571371.
102. Liang-Yu C, Kuo S C, Liu C Y, Luo B S, Huang L J, Lee Y T, Chen C P, Chen T L, Fung C P. Difference in imipenem, meropenem, sulbactam, and colistin nonsusceptibility trends among three phenotypically undifferentiated Acinetobacter baumannii complex in a medical center in Taiwan, 1997-2007. J Microbiol Immunol Infect. 2011; 44(5):358-63. doi: 10.1016/j.jmii.2011.01.032. PubMed PMID: 21524973.
103. Hindler J A, Humphries R M. Colistin M I C variability by method for contemporary clinical isolates of multidrug-resistant Gram-negative bacilli. J Clin Microbiol. 2013; 51(6):1678-84. doi: 10.1128/JCM.03385-12. PubMed PMID: 23486719; PMCID: PMC3716094.
104. Rivera C G, Narayanan P P, Patel R, Estes L L. Impact of Cefepime Susceptible-Dose-Dependent MIC for Enterobacteriaceae on Reporting and Prescribing. Antimicrob Agents Chemother. 2016; 60(6):3854-5. doi: 10.1128/AAC.00442-16. PubMed PMID: 27067319; PMCID: PMC4879407.
105. Dervan P B, Becker, MM. Molecular recognition of DNA by small molecules. Synthesis of bis(methidium)spermine, a DNA polyintercalating molecule. 1978; 100(6):1968-1970 106. Rajecky, M (2015) Interaction of benzo[c]phenanthridine alkaloids with DNA. Retrieved from Masaryk University.
107. Le T N, et al., A Versatile Total Synthesis of Benzo[c]phenanthridine and Protoberberine Alkaloids Using Lithiated Toluamide-Benzonitrile Cycloaddition. 2004; 69(8):2768-2772.
108. Michael A J. Polyamines in Eukaryotes, Bacteria, and Archaea. J Biol Chem. 2016; 291(29):14896-903. doi: 10.1074/jbc.R116.734780. PubMed PMID: 27268252; PMCID: PMC4946907.
109. D'Agostino L, di Pietro M, Di Luccia A. Nuclear aggregates of polyamines are supramolecular structures that play a crucial role in genomic DNA protection and conformation. FEBS J. 2005; 272(15):3777-87. doi: 10.1111/j.1742-4658.2005.04782.x. PubMed PMID: 16045750.
110. Miller-Fleming L, Olin-Sandoval V, Campbell K, Ralser M. Remaining Mysteries of Molecular Biology: The Role of Polyamines in the Cell. J Mol Biol. 2015; 427(21):3389-406. doi: 10.1016/jjmb.2015.06.020. PubMed PMID: 26156863.
111. Michael A J. Biosynthesis of polyamines and polyamine-containing molecules. Biochem J. 2016; 473(15):2315-29. doi: 10.1042/BCJ20160185. PubMed PMID: 27470594.
112. D'Agostino L, di Pietro M, Di Luccia A. Nuclear aggregates of polyamines. IUBMB Life. 2006; 58(2):75-82. doi: 10.1080/15216540600662525. PubMed PMID: 16608821.
113. Karatan E, Michael A J. A wider role for polyamines in biofilm formation. Biotechnol Lett. 2013; 35(11):1715-7. doi: 10.1007/s10529-013-1286-3. PubMed PMID: 23881324.
114. McGinnis M W, Parker Z M, Walter N E, Rutkovsky A C, Cartaya-Marin C, Karatan E. Spermidine regulates Vibrio cholerae biofilm formation via transport and signaling pathways. FEMS Microbiol Lett. 2009; 299(2):166-74. doi: 10.1111/j.1574-6968.2009.01744.x. PubMed PMID: 19694812.
115. Wortham B W, Oliveira M A, Fetherston J D, Perry R D. Polyamines are required for the expression of key Hms proteins important for Yersinia pestis biofilm formation. Environ Microbiol. 2010; 12(7):2034-47. doi: 10.1111/j.1462-2920.2010.02219.x. PubMed PMID: 20406298; PMCID: PMC3039482.
116. Goytia M, Dhulipala V L, Shafer W M. Spermine impairs biofilm formation by Neisseria gonorrhoeae. FEMS Microbiol Lett. 2013; 343(1):64-9. doi: 10.1111/1574-6968.12130. PubMed PMID: 23506248; PMCID: PMC3651792.
117. Cardile A P, Woodbury R L, Sanchez C J, Jr., Becerra S C, Garcia R A, Mende K, Wenke J C, Akers K S. Activity of Norspermidine on Bacterial Biofilms of Multidrug-Resistant Clinical Isolates Associated with Persistent Extremity Wound Infections. Adv Exp Med Biol. 2017; 973:53-70. doi: 10.1007/5584_2016_93. PubMed PMID: 27864804; PMCID: PMC5438303.
118. Wang Y, Kim S H, Natarajan R, Heindl J E, Bruger E L, Waters C M, Michael A J, Fuqua C. Spermidine Inversely Influences Surface Interactions and Planktonic Growth in Agrobacterium tumefaciens. J Bacteriol. 2016; 198(19):2682-91. doi: 10.1128/JB.00265-16. PubMed PMID: 27402627; PMCID: PMC5019071.
119. Qu L, She P, Wang Y, Liu F, Zhang D, Chen L, Luo Z, Xu H, Qi Y, Wu Y. Effects of norspermidine on Pseudomonas aeruginosa biofilm formation and eradication. Microbiologyopen. 2016; 5(3):402-12. doi: 10.1002/mbo3.338. PubMed PMID: 26817804; PMCID: PMC4905993.
120. Konai M M, Adhikary U, Samaddar S, Ghosh C, Haldar J. Structure-Activity Relationship of Amino Acid Tunable Lipidated Norspermidine Conjugates: Disrupting Biofilms with Potent Activity against Bacterial Persisters. Bioconjug Chem. 2015; 26(12):2442-53. doi: 10.1021/acs.bioconjchem.5b00494. PubMed PMID: 26452096.
121. Si X, Quan X, Wu Y. A small-molecule norspermidine and norspermidine-hosting polyelectrolyte coatings inhibit biofilm formation by multi-species wastewater culture. Appl Microbiol Biotechnol. 2015; 99(24):10861-70. doi: 10.1007/s00253-015-6943-0. PubMed PMID: 26350146.
122. Dewangan R P, Joshi S, Kumari S, Gautam H, Yar M S, Pasha S. N-terminally modified linear and branched spermine backbone dipeptidomimetics against planktonic and sessile methicillin-resistant Staphylococcus aureus. Antimicrob Agents Chemother. 2014; 58(9):5435-47. doi: 10.1128/AAC.03391-14. PubMed PMID: 24982082; PMCID: PMC4135887.
123. Ding Y, Peng N, Du Y, Ji L, Cao B. Disruption of putrescine biosynthesis in Shewanella oneidensis enhances biofilm cohesiveness and performance in Cr(VI) immobilization. Appl Environ Microbiol. 2014; 80(4):1498-506. doi: 10.1128/AEM.03461-13. PubMed PMID: 24362428; PMCID: PMC3911039.
124. Planet P J, LaRussa S J, Dana A, Smith H, Xu A, Ryan C, Uhlemann A C, Boundy S, Goldberg J, Narechania A, Kulkarni R, Ratner A J, Geoghegan J A, Kolokotronis S O, Prince A. Emergence of the epidemic methicillin-resistant Staphylococcus aureus strain USA300 coincides with horizontal transfer of the arginine catabolic mobile element and speG-mediated adaptations for survival on skin. MBio. 2013; 4(6):e00889-13. doi: 10.1128/mBio.00889-13. PubMed PMID: 24345744; PMCID: PMC3870260.
125. Hobley L, Li B, Wood J L, Kim S H, Naidoo J, Ferreira A S, Khomutov M, Khomutov A, Stanley-Wall N R, Michael A J. Spermidine promotes Bacillus subtilis biofilm formation by activating expression of the matrix regulator slrR. J Biol Chem. 2017; 292(29):12041-53. doi: 10.1074/jbc.M117.789644. PubMed PMID: 28546427; PMCID: PMC5519356.
126. Ou M, Ling J. Norspermidine changes the basic structure of S. mutans biofilm. Mol Med Rep. 2017; 15(1):210-20. doi: 10.3892/mmr.2016.5979. PubMed PMID: 27922663; PMCID: PMC5355703.
127. Nesse L L, Berg K, Vestby L K. Effects of norspermidine and spermidine on biofilm formation by potentially pathogenic Escherichia coli and Salmonella enterica wild-type strains. Appl Environ Microbiol. 2015; 81(6):2226-32. doi: 10.1128/AEM.03518-14. PubMed PMID: 25595767; PMCID: PMC4345383.
128. Ramon-Perez M L, Diaz-Cedillo F, Contreras-Rodriguez A, Betanzos-Cabrera G, Peralta H, Rodriguez-Martinez S, Cancino-Diaz M E, Jan-Roblero J, Cancino Diaz J C. Different sensitivity levels to norspermidine on biofilm formation in clinical and commensal Staphylococcus epidermidis strains. Microb Pathog. 2015; 79:8-16. doi: 10.1016/j.micpath.2014.12.004. PubMed PMID: 25549879.
129. Hobley L, Kim S H, Maezato Y, Wyllie S, Fairlamb A H, Stanley-Wall N R, Michael A J. Norspermidine is not a self-produced trigger for biofilm disassembly. Cell. 2014; 156(4):844-54. doi: 10.1016/j.cell.2014.01.012. PubMed PMID: 24529384; PMCID: PMC3969229.
130. Sakamoto A, Terui Y, Yamamoto T, Kasahara T, Nakamura M, Tomitori H, Yamamoto K, Ishihama A, Michael A J, Igarashi K, Kashiwagi K. Enhanced biofilm formation and/or cell viability by polyamines through stimulation of response regulators UvrY and CpxR in the two-component signal transducing systems, and ribosome recycling factor. Int J Biochem Cell Biol. 2012; 44(11):1877-86. doi: 10.1016/j.bioce1.2012.07.010. PubMed PMID: 22814172.
131. Burrell M, Hanfrey C C, Murray E J, Stanley-Wall N R, Michael A J. Evolution and multiplicity of arginine decarboxylases in polyamine biosynthesis and essential role in Bacillus subtilis biofilm formation. J Biol Chem. 2010; 285(50):39224-38. doi: 10.1074/jbc.M110.163154. PubMed PMID: 20876533; PMCID: PMC2998088.
132. Lee J, Sperandio V, Frantz D E, Longgood J, Camilli A, Phillips M A, Michael A J. An alternative polyamine biosynthetic pathway is widespread in bacteria and essential for biofilm formation in Vibrio cholerae. J Biol Chem. 2009; 284(15):9899-907. doi: 10.1074/jbc.M900110200. PubMed PMID: 19196710; PMCID: PMC2665113.
133. Patel C N, Wortham B W, Lines J L, Fetherston J D, Perry R D, Oliveira M A. Polyamines are essential for the formation of plague biofilm. J Bacteriol. 2006; 188(7):2355-63. doi: 10.1128/JB.188.7.2355-2363.2006. PubMed PMID: 16547021; PMCID: PMC1428407.
134. Chen X, Wu H, Cao Y, Yao X, Zhao L, Wang T, Yang Y, Lv D, Chai Y, Cao Y, Zhu Z. Ion-pairing chromatography on a porous graphitic carbon column coupled with time-of-flight mass spectrometry for targeted and untargeted profiling of amino acid biomarkers involved in Candida albicans biofilm formation. Mol Biosyst. 2014; 10(1):74-85. doi: 10.1039/c3mb70240e. PubMed PMID: 24150280.
135. Liao Z, ZhangGuan X, Zhu Z, Yao X, Yang Y, Jiang Y, Cao Y. Enhancement of the antibiofilm activity of amphotericin B by polyamine biosynthesis inhibitors. Int J Antimicrob Agents. 2015; 46(1):45-52. doi: 10.1016/j.ijantimicag.2015.02.021. PubMed PMID: 25937097.
136. Visick K L, Schembri M A, Yildiz F, Ghigo J M. Biofilms 2015: Multidisciplinary Approaches Shed Light into Microbial Life on Surfaces. J Bacteriol. 2016; 198(19):2553-63. doi: 10.1128/JB.00156-16. PubMed PMID: 26977109; PMCID: PMC5019050.
137. Hoiby N. A short history of microbial biofilms and biofilm infections. APMIS. 2017; 125(4):272-5. doi: 10.1111/apm.12686. PubMed PMID: 28407426.
138. Gunn J S, Bakaletz L O, Wozniak D J. What's on the Outside Matters: The Role of the Extracellular Polymeric Substance of Gram-negative Biofilms in Evading Host Immunity and as a Target for Therapeutic Intervention. J Biol Chem. 2016; 291(24):12538-46. doi: 10.1074/jbc.R115.707547. PubMed PMID: 27129225; PMCID: PMC4933457.
139. Martins M, Uppuluri P, Thomas D P, Cleary I A, Henriques M, Lopez-Ribot J L, Oliveira R. Presence of extracellular DNA in the Candida albicans biofilm matrix and its contribution to biofilms. Mycopathologia. 2010; 169(5):323-31. doi: 10.1007/s11046-009-9264-y. PubMed PMID: 20012895; PMCID: PMC3973130.
140. Brandstetter K A, Jurcisek J A, Goodman S D, Bakaletz L O, Das S. Antibodies directed against integration host factor mediate biofilm clearance from Nasopore. Laryngoscope. 2013; 123(11):2626-32. doi: 10.1002/lary.24183. PubMed PMID: 23670606; PMCID: PMC4060527.
141. Brockson M E, Novotny L A, Mokrzan E M, Malhotra S, Jurcisek J A, Akbar R, Devaraj A, Goodman S D, Bakaletz L O. Evaluation of the kinetics and mechanism of action of anti-integration host factor-mediated disruption of bacterial biofilms. Mol Microbiol. 2014; 93(6):1246-58. doi: 10.1111/mmi.12735. PubMed PMID: 25069521; PMCID: PMC4160410.
142. Devaraj A, Justice S S, Bakaletz L O, Goodman S D. DNABII proteins play a central role in UPEC biofilm structure. Mol Microbiol. 2015; 96(6):1119-35. doi: 10.1111/mmi.12994. PubMed PMID: 25757804; PMCID: PMC4464964.
143. Freire M O, Devaraj A, Young A, Navarro J B, Downey J S, Chen C, Bakaletz L O, Zadeh H H, Goodman S D. A bacteriuml-biofilm-induced oral osteolytic infection can be successfully treated by immuno-targeting an extracellular nucleoid-associated protein. Mol Oral Microbiol. 2017; 32(1):74-88. doi: 10.1111/omi.12155. PubMed PMID: 26931773; PMCID: PMC5010536.
144. Gustave J E, Jurcisek J A, McCoy K S, Goodman S D, Bakaletz L O. Targeting bacterial integration host factor to disrupt biofilms associated with cystic fibrosis. J Cyst Fibros. 2013; 12(4):384-9. doi: 10.1016/j.jcf.2012.10.011. PubMed PMID: 23168017; PMCID: PMC3582735.
145. Novotny L A, Clements J D, Goodman S D, Bakaletz L O. Transcutaneous Immunization with a Band-Aid Prevents Experimental Otitis Media in a Polymicrobial Model. Clin Vaccine Immunol. 2017; 24(6). doi: 10.1128/CVI.00563-16. PubMed PMID: 28381402; PMCID: PMC5461379.
146. Novotny L A, Jurcisek J A, Goodman S D, Bakaletz L O. Monoclonal antibodies against DNA-binding tips of DNABII proteins disrupt biofilms in vitro and induce bacterial clearance in vivo. EBioMedicine. 2016; 10:33-44. doi: 10.1016/j.ebiom.2016.06.022. PubMed PMID: 27342872; PMCID: PMC5006588.
147. Novotny L A, Jurcisek J A, Ward M O, Jr., Jordan Z B, Goodman S D, Bakaletz L O. Antibodies against the majority subunit of type IV Pili disperse nontypeable Haemophilus influenzae biofilms in a LuxS-dependent manner and confer therapeutic resolution of experimental otitis media. Mol Microbiol. 2015; 96(2):276-92. doi: 10.1111/mmi.12934. PubMed PMID: 25597921; PMCID: PMC4423401.
148. Rocco C J, Davey M E, Bakaletz L O, Goodman S D. Natural antigenic differences in the functionally equivalent extracellular DNABII proteins of bacterial biofilms provide a means for targeted biofilm therapeutics. Mol Oral Microbiol. 2017; 32(2):118-30. doi: 10.1111/omi.12157. PubMed PMID: 26988714; PMCID: PMC5023461.
149. Novotny L A, Amer A O, Brockson M E, Goodman S D, Bakaletz L O. Structural stability of Burkholderia cenocepacia biofilms is reliant on eDNA structure and presence of a bacteriuml nucleic acid binding protein. PLoS One. 2013; 8(6):e67629. doi: 10.1371/journal.pone.0067629. PubMed PMID: 23799151; PMCID: PMC3682984.
150. Baelo A, Levato R, Julian E, Crespo A, Astola J, Gavalda J, Engel E, Mateos-Timoneda M A, Torrents E. Disassembling bacterial extracellular matrix with DNase-coated nanoparticles to enhance antibiotic delivery in biofilm infections. J Control Release. 2015; 209:150-8. doi: 10.1016/j.jconre1.2015.04.028. PubMed PMID: 25913364.
151. Brown H L, Hanman K, Reuter M, Betts R P, van Vliet A H. Campylobacter jejuni biofilms contain extracellular DNA and are sensitive to DNase I treatment. Front Microbiol. 2015; 6:699. doi: 10.3389/fmicb.2015.00699. PubMed PMID: 26217328; PMCID: PMC4498105.
152. Frederiksen B, Pressler T, Hansen A, Koch C, Hoiby N. Effect of aerosolized rhDNase (Pulmozyme) on pulmonary colonization in patients with cystic fibrosis. Acta Paediatr. 2006; 95(9):1070-4. doi: 10.1080/08035250600752466. PubMed PMID: 16938752.
153. Martins M, Henriques M, Lopez-Ribot J L, Oliveira R. Addition of DNase improves the in vitro activity of antifungal drugs against Candida albicans biofilms. Mycoses. 2012; 55(1):80-5. doi: 10.1111/j.1439-0507.2011.02047.x. PubMed PMID: 21668524; PMCID: PMC3175262.
154. Goodman S D, Obergfell K P, Jurcisek J A, Novotny L A, Downey J S, Ayala E A, Tjokro N, Li B, Justice S S, Bakaletz L O. Biofilms can be dispersed by focusing the immune system on a common family of bacterial nucleoid-associated proteins. Mucosal Immunol. 2011; 4(6):625-37. doi: 10.1038/mi.2011.27. PubMed PMID: 21716265.
155. Pasini A, Caldarera C M, Giordano E. Chromatin remodeling by polyamines and polyamine analogs. Amino Acids. 2014; 46(3):595-603. doi: 10.1007/s00726-013-1550-9. PubMed PMID: 23836422.
156. Baeza I, Ibanez M, Wong C, Chavez P, Gariglio P, Oro J. Possible prebiotic significance of polyamines in the condensation, protection, encapsulation, and biological properties of DNA. Orig Life Evol Biosph. 1991; 21(4):225-42. PubMed PMID: 1668678.
157. Nayvelt I, Hyvonen M T, Alhonen L, Pandya I, Thomas T, Khomutov A R, Vepsalainen J, Patel R, Keinanen T A, Thomas T J. DNA condensation by chiral alpha-methylated polyamine analogues and protection of cellular DNA from oxidative damage. Biomacromolecules. 2010; 11(1):97-105. doi: 10.1021/bm900958c. PubMed PMID: 19919070.
158. Cavaliere R., Ball J. L., Turnbull L., and Whitchurch C. (2014). The biofilm matri destabilizers, EDTA, and DNAseI, enhance biofilms to treatment with ampicillin and ciprofloxacin. Microbiology Open. 3(4):557-567.
159. Chmielewski R. A. N. and Frank J. F. (2003). Biofilm Formation and Control in Food Processing Facilities. Comprehensive Reviews in Food Science and Food Safety. Vol. 2. 22-32.
160. Devaraj A., Justice S. S., Bakaletz L. O., and Goodman S. D. (2015). DNABII Proteins play a central role in UPEC biofilm structure. Molecular Microbiology. 96 (6): 1119-1135.
161. Dongari-Bagtzoglou A. (2008). Pathogenesis of mucosal biofilm infections: challenges and progress. Expert Rev Anti Infect Ther.6(2):201-8. Donlan R. M. (2001). Biofilms and device-associated infections. Emerging Infectious Diseases. 7(2):277-281.
162. Flemming H. and Wingender J. (2010). The biofilm matrix. Nature Reviews Microbiology. 8:623-633 163. Goodman S. D., Obergfell K. P., Jurcisek J. A., Novotny L. A., Downey J. S., Ayala E. A., Tjokro N., Li B., Justice S. S., and Bakaletz L. O. (2011). Mucosal Immunology. 4 (6): 625-637.
164. Gueroult M., Boittin O., Mauffret O., Etchebest C., and Hartmann B. (2012). Mg 2+ in the Major Groove Modulates B-DNA Structure and Dynamics. PLOS ONE. (7)-7-e41704.
165. Hackl E. V., Kornilova S. V., and Blagoi Y. P. (2005) DNA structural transitions induced by divalent metals ions in aqueous solutions. International Journal of Biological Macromolecules 35:175-191.
166. Hobley L., Li B., Wood J. L., Kim S. H., Naidoo J., Ferreira A. S., Khomutov M., Khomutov A., Stanley-Wall N. R., and Michael A. J. (2017). Spermidine promotoes Bacillus subtilis biofilm formation by activating expression of the matrix regulator slrR. Journal of Biological Chemistry. 292(29): 12041-12053.
167. Nash H. A., Robertson C. A., Flamm E., Weisberg R. A., and Miller H. I. (1987). Overproduction of Escherichia coli Integration Host Factor, a Protein with Nonidentical Subunits. Journal of Bacteriology. 4124-4127.
168. Novotny L. A., Jucisek J. A., Goodman S. D., and Bakaletz L. O. (2016). Monoclonal antibodies against DNA-binding tips of DNABII proteins disrupt biofilms in vitro and induce bacterial clearance in vivo. EBioMedicine. (10):33-44.
169. Patel C. N., Wortham B. W., Lines J. L., Fetherston J. D., Perry R. D., and Oliveira M. A. (2006). Polyamines Are Essential for the Formation of Plague Biofilm. Journal of Bacteriology. 2355-2363.
170. Tan Z-J. and Chen S-J. (2006). Nucleic Acid Helix Stability: Effects of Salt Concentration, Cation Valence and Size, and Chain Length. Biophysical Journal. (90): 1175-1190.
171. Vorgias C. E., and Wilson K. S. (1991). A rapid purification procedure of recombinant integration host factor from Escherichia coli. Protein Expression and Purifcation. 2(5-6):317-20.
172. Wood T. L., Guha R., Tang. L., Geitner M., Kumar M., and Wood T. K. (2016). Living biofouling-resistant membranes as a model for the beneficial use of engineered biofilms. PNAS. E2802-E2811.
173. Visick K L, Schembri M A, Yildiz F, Ghigo J M. Biofilms 2015: Multidisciplinary Approaches Shed Light into Microbial Life on Surfaces. J Bacteriol. 2016; 198(19):2553-63. doi: 10.1128/JB.00156-16. PubMed PMID: 26977109; PMCID: PMC5019050.
174. Hoiby N. A short history of microbial biofilms and biofilm infections. APMIS. 2017; 125(4):272-5. doi: 10.1111/apm.12686. PubMed PMID: 28407426.
175. Gunn J S, Bakaletz L O, Wozniak D J. What's on the Outside Matters: The Role of the Extracellular Polymeric Substance of Gram-negative Biofilms in Evading Host Immunity and as a Target for Therapeutic Intervention. J Biol Chem. 2016; 291(24):12538-46. doi: 10.1074/jbc.R115.707547. PubMed PMID: 27129225; PMCID: PMC4933457.
176. Okshevsky M, Meyer R L. The role of extracellular DNA in the establishment, maintenance and perpetuation of bacterial biofilms. Crit Rev Microbiol. 2015; 41(3):341-52. doi: 10.3109/1040841X.2013.841639. PubMed PMID: 24303798.
177. Wilton M, Charron-Mazenod L, Moore R, Lewenza S. Extracellular DNA Acidifies Biofilms and Induces Aminoglycoside Resistance in Pseudomonas aeruginosa. Antimicrob Agents Chemother. 2015; 60(1):544-53. doi: 10.1128/AAC.01650-15. PubMed PMID: 26552982; PMCID: PMC4704225.
178. Brandstetter K A, Jurcisek J A, Goodman S D, Bakaletz L O, Das S. Antibodies directed against integration host factor mediate biofilm clearance from Nasopore. Laryngoscope. 2013; 123(11):2626-32. doi: 10.1002/lary.24183. PubMed PMID: 23670606; PMCID: PMC4060527.
179. Brockson M E, Novotny L A, Mokrzan E M, Malhotra S, Jurcisek J A, Akbar R, Devaraj A, Goodman S D, Bakaletz L O. Evaluation of the kinetics and mechanism of action of anti-integration host factor-mediated disruption of bacterial biofilms. Mol Microbiol. 2014; 93(6):1246-58. doi: 10.1111/mmi.12735. PubMed PMID: 25069521; PMCID: PMC4160410.
180. Devaraj A, Justice S S, Bakaletz L O, Goodman S D. DNABII proteins play a central role in UPEC biofilm structure. Mol Microbiol. 2015; 96(6):1119-35. doi: 10.1111/mmi.12994. PubMed PMID: 25757804; PMCID: PMC4464964.
181. Freire M O, Devaraj A, Young A, Navarro J B, Downey J S, Chen C, Bakaletz L O, Zadeh H H, Goodman S D. A bacteriuml-biofilm-induced oral osteolytic infection can be successfully treated by immuno-targeting an extracellular nucleoid-associated protein. Mol Oral Microbiol. 2017; 32(1):74-88. doi: 10.1111/omi.12155. PubMed PMID: 26931773; PMCID: PMC5010536.
182. Gustave J E, Jurcisek J A, McCoy K S, Goodman S D, Bakaletz L O. Targeting bacterial integration host factor to disrupt biofilms associated with cystic fibrosis. J Cyst Fibros. 2013; 12(4):384-9. doi: 10.1016/j.jcf.2012.10.011. PubMed PMID: 23168017; PMCID: PMC3582735.
183. Novotny L A, Clements J D, Goodman S D, Bakaletz L O. Transcutaneous Immunization with a Band-Aid Prevents Experimental Otitis Media in a Polymicrobial Model. Clin Vaccine Immunol. 2017; 24(6). doi: 10.1128/CVI.00563-16. PubMed PMID: 28381402; PMCID: PMC5461379.
184. Novotny L A, Jurcisek J A, Goodman S D, Bakaletz L O. Monoclonal antibodies against DNA-binding tips of DNABII proteins disrupt biofilms in vitro and induce bacterial clearance in vivo. EBioMedicine. 2016; 10:33-44. doi: 10.1016/j.ebiom.2016.06.022. PubMed PMID: 27342872; PMCID: PMC5006588.
185. Rocco C J, Davey M E, Bakaletz L O, Goodman S D. Natural antigenic differences in the functionally equivalent extracellular DNABII proteins of bacterial biofilms provide a means for targeted biofilm therapeutics. Mol Oral Microbiol. 2017; 32(2):118-30. doi: 10.1111/omi.12157. PubMed PMID: 26988714; PMCID: PMC5023461.
186. Novotny L A, Amer A O, Brockson M E, Goodman S D, Bakaletz L O. Structural stability of Burkholderia cenocepacia biofilms is reliant on eDNA structure and presence of a bacteriuml nucleic acid binding protein. PLoS One. 2013; 8(6):e67629. doi: 10.1371/journal.pone.0067629. PubMed PMID: 23799151; PMCID: PMC3682984.
187. Baelo A, Levato R, Julian E, Crespo A, Astola J, Gavalda J, Engel E, Mateos-Timoneda M A, Torrents E. Disassembling bacterial extracellular matrix with DNase-coated nanoparticles to enhance antibiotic delivery in biofilm infections. J Control Release. 2015; 209:150-8. doi: 10.1016/j.jconre1.2015.04.028. PubMed PMID: 25913364.
188. Brown H L, Hanman K, Reuter M, Betts R P, van Vliet A H. Campylobacter jejuni biofilms contain extracellular DNA and are sensitive to DNase I treatment. Front Microbiol. 2015; 6:699. doi: 10.3389/fmicb.2015.00699. PubMed PMID: 26217328; PMCID: PMC4498105.
189. Frederiksen B, Pressler T, Hansen A, Koch C, Hoiby N. Effect of aerosolized rhDNase (Pulmozyme) on pulmonary colonization in patients with cystic fibrosis. Acta Paediatr. 2006; 95(9):1070-4. doi: 10.1080/08035250600752466. PubMed PMID: 16938752.
190. Martins M, Henriques M, Lopez-Ribot J L, Oliveira R. Addition of DNase improves the in vitro activity of antifungal drugs against Candida albicans biofilms. Mycoses. 2012; 55(1):80-5. doi: 10.1111/j.1439-0507.2011.02047.x. PubMed PMID: 21668524; PMCID: PMC3175262.
191. Goodman S D, Obergfell K P, Jurcisek J A, Novotny L A, Downey J S, Ayala E A, Tjokro N, Li B, Justice S S, Bakaletz L O. Biofilms can be dispersed by focusing the immune system on a common family of bacterial nucleoid-associated proteins. Mucosal Immunol. 2011; 4(6):625-37. doi: 10.1038/mi.2011.27. PubMed PMID: 21716265.
192. Cho C, Chande A, Gakhar L, Bakaletz L O, Jurcisek J A, Ketterer M, Shao J, Gotoh K, Foster E, Hunt J, O'Brien E, Apicella M A. Role of the nuclease of nontypeable Haemophilus influenzae in dispersal of organisms from biofilms. Infect Immun. 2015; 83(3):950-7. doi: 10.1128/IAI.02601-14. PubMed PMID: 25547799; PMCID: PMC4333478.
193. Kiedrowski M R, Kavanaugh J S, Malone C L, Mootz J M, Voyich J M, Smeltzer M S, Bayles K W, Horswill A R. Nuclease modulates biofilm formation in community-associated methicillin-resistant Staphylococcus aureus. PLoS One. 2011; 6(11):e26714. doi: 10.1371/journal.pone.0026714. PubMed PMID: 22096493; PMCID: PMC3214024.
194. Liu J, Sun L, Liu W, Guo L, Liu Z, Wei X, Ling J. A Nuclease from Streptococcus mutans Facilitates Biofilm Dispersal and Escape from Killing by Neutrophil Extracellular Traps. Front Cell Infect Microbiol. 2017; 7:97. doi: 10.3389/fcimb.2017.00097. PubMed PMID: 28401067; PMCID: PMC5368189.
195. Steichen C T, Cho C, Shao J Q, Apicella M A. The Neisseria gonorrhoeae biofilm matrix contains DNA, and an endogenous nuclease controls its incorporation. Infect Immun. 2011; 79(4):1504-11. doi: 10.1128/IAI.01162-10. PubMed PMID: 21300774; PMCID: PMC3067525.
196. Hall-Stoodley L, Nistico L, Sambanthamoorthy K, Dice B, Nguyen D, Mershon W J, Johnson C, Hu F Z, Stoodley P, Ehrlich G D, Post J C. Characterization of biofilm matrix, degradation by DNase treatment and evidence of capsule downregulation in Streptococcus pneumoniae clinical isolates. BMC Microbiol. 2008; 8:173. doi: 10.1186/1471-2180-8-173. PubMed PMID: 18842140; PMCID: PMC2600794.
197. Izano E A, Shah S M, Kaplan J B. Intercellular adhesion and biocide resistance in nontypeable Haemophilus influenzae biofilms. Microb Pathog. 2009; 46(4):207-13. doi: 10.1016/j.micpath.2009.01.004. PubMed PMID: 19490830; PMCID: PMC2691864.
198. Kaplan J B, LoVetri K, Cardona S T, Madhyastha S, Sadovskaya I, Jabbouri S, Izano E A. Recombinant human DNase I decreases biofilm and increases antimicrobial susceptibility in staphylococci. J Antibiot (Tokyo). 2012; 65(2):73-7. doi: 10.1038/ja.2011.113. PubMed PMID: 22167157; PMCID: PMC3288126.

199. Tetz V V, Tetz G V. Effect of extracellular DNA destruction by DNase I on characteristics of forming biofilms. DNA Cell Biol. 2010; 29(8):399-405. doi: 10.1089/dna.2009.1011. PubMed PMID: 20491577.
200. Wang G, Vasquez K M. Z-DNA, an active element in the genome. Front Biosci. 2007; 12:4424-38. PubMed PMID: 17485386.
201. Barraud P, Allain F H. ADAR proteins: double-stranded RNA and Z-DNA binding domains. Curr Top Microbiol Immunol. 2012; 353:35-60. doi: 10.1007/82_2011_145. PubMed PMID: 21728134; PMCID: PMC3226063.
202. Choi J, Majima T. Conformational changes of non-B DNA. Chem Soc Rev. 2011; 40(12):5893-909. doi: 10.1039/cics15153c. PubMed PMID: 21901191.
203. Yang L, Wang S, Tian T, Zhou X. Advancements in Z-DNA: development of inducers and stabilizers for B to Z transition. Curr Med Chem. 2012; 19(4):557-68. PubMed PMID: 22204344.
204. D'Agostino L, di Pietro M, Di Luccia A. Nuclear aggregates of polyamines. IUBMB Life. 2006; 58(2):75-82. doi: 10.1080/15216540600662525. PubMed PMID: 16608821.

205. Balasundaram D, Tyagi A K. Polyamine-DNA nexus: structural ramifications and biological implications. Mol Cell Biochem. 1991; 100(2):129-40. PubMed PMID: 2008175.

206. Thomas T J, Gunnia U B, Thomas T. Polyamine-induced B-DNA to Z-DNA conformational transition of a plasmid DNA with (dG-dC)n insert. J Biol Chem. 1991; 266(10):6137-41. PubMed PMID: 1848849.
207. Kim S K, Eriksson S, Norden B. Z->B transition in poly[d(G-m5C)2] induced by interaction with 4′,6-diamidino-2-phenylindole. Biopolymers. 1993; 33(11):1677-86. doi: 10.1002/bip.360331105. PubMed PMID: 8241427.
208. Mirau P A, Kearns D R. The effect of intercalating drugs on the kinetics of the B to Z transition of poly(dG-dC). Nucleic Acids Res. 1983; 11(6):1931-41. PubMed PMID: 6835844; PMCID: PMC325847.
209. Waga S, Mizuno S, Yoshida M. Nonhistone protein HMG1 removes the transcriptional block caused by left-handed Z-form segment in a supercoiled DNA. Biochem Biophys Res Commun. 1988; 153(1):334-9. PubMed PMID: 3377790.
210. Pasini A, Caldarera C M, Giordano E. Chromatin remodeling by polyamines and polyamine analogs. Amino Acids. 2014; 46(3):595-603. doi: 10.1007/s00726-013-1550-9. PubMed PMID: 23836422.
211. Baeza I, Ibanez M, Wong C, Chavez P, Gariglio P, Oro J. Possible prebiotic significance of polyamines in the condensation, protection, encapsulation, and biological properties of DNA. Orig Life Evol Biosph. 1991; 21(4):225-42. PubMed PMID: 1668678.
212. D'Agostino L, di Pietro M, Di Luccia A. Nuclear aggregates of polyamines are supramolecular structures that play a crucial role in genomic DNA protection and conformation. FEBS J. 2005; 272(15):3777-87. doi: 10.1111/j.1742-4658.2005.04782.x. PubMed PMID: 16045750.
213. Nayvelt I, Hyvonen M T, Alhonen L, Pandya I, Thomas T, Khomutov A R, Vepsalainen J, Patel R, Keinanen T A, Thomas T J. DNA condensation by chiral alpha-methylated polyamine analogues and protection of cellular DNA from oxidative damage. Biomacromolecules. 2010; 11(1):97-105. doi: 10.1021/bm900958c. PubMed PMID: 19919070.
214. Duell B L, Su Y C, Riesbeck K. Host-pathogen interactions of nontypeable Haemophilus influenzae: from commensal to pathogen. FEBS Lett. 2016; 590(21):3840-53. doi: 10.1002/1873-3468.12351. PubMed PMID: 27508518.
215. Blango M G, Mulvey M A. Persistence of uropathogenic Escherichia coli in the face of multiple antibiotics. Antimicrob Agents Chemother. 2010; 54(5):1855-63. doi: 10.1128/AAC.00014-10. PubMed PMID: 20231390; PMCID: PMC2863638.
216. Jang Y J, Lee C, Kim S K. Formation of Poly[d(A-T)2] Specific Z-DNA by a Cationic Porphyrin. Sci Rep. 2015; 5:9943. doi: 10.1038/srep09943. PubMed PMID: 25943171; PMCID: PMC4421867.

1. A method for inhibiting the stability of a biofilm, comprising contacting the biofilm with an effective amount of one or more of the following:
(a) an agent that interferes with the binding of a polyamine to DNA in the biofilm, wherein the agent is not an HMGB1 protein, fragment or an equivalent of each thereof;
(b) an that interferes with the binding of a polyamine to DNA in the biofilm, wherein the agent is not an HMGB1 protein, fragment or an equivalent of each thereof;
(c) an HMGB1 protein or a biologically active fragment thereof and anti-B-DNA antibody or fragment or derivative thereof, wherein the contacting comprises coating a surface ex vivo with an effective amount of the HMGB1 protein or biologically active fragment thereof and the anti-B-DNA antibody or fragment or derivative thereof; or
(d) chloroquine and anti-B-DNA antibody or a fragment or derivative thereof, wherein the contacting comprises coating a surface ex vivo with an effective amount of chloroquine and anti-B-DNA antibody or a fragment or derivative thereof.

2. A method for treating a biofilm in a subject, comprising administering to the subject infected with a biofilm an effective amount of one or more agents that interfere with the binding of a polyamine to the DNA in the biofilm, wherein the agent is not an HMGB1 protein, a fragment thereof or an equivalent of each thereof.

3. A method for preventing the formation of a biofilm in a subject susceptible to developing a biofilm, comprising administering to the subject an effective amount of one or more agents that interfere with the binding of a polyamine to the DNA in the biofilm, optionally wherein the agent is not an HMGB1 protein, a fragment thereof or an equivalent of each thereof.

4. A method for treating an infection caused by a bacterium that produces a biofilm in a subject in need thereof, the method comprising administering to the subject an effective amount of one or more agents that interfere with the binding of a polyamine to the DNA in the biofilm and an agent that inhibits the replication of the organism, optionally wherein the agent is not an HMGB1 protein, a fragment thereof or an equivalent of each thereof.

5-9. (canceled)

10. The method of claim 1, wherein one or more of the following applies:
(i) the agent that interferes with the binding of a polyamine to DNA in the biofilm is a tRNA;
(ii) the agent is an inhibitor of polyamine synthesis or an agent that inhibits the binding of the polyamine to the DNA, wherein the agent is not an HMGB1 protein, fragment or an equivalent of each thereof;
(iii) the polyamine is selected from the group of: putrescine, spermine, cadaverine, 1,3-diaminopropane or spermidine;
(iv) the agent comprises a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof;
(v) the agent comprises an agent that depletes cations from the biofilm, optionally a cation exchange resin, an aminopolycarboxylic acid, a crown ether, an azacrown, or a cryptand, and optionally wherein the agent that depletes cations from the biofilm are from the group of: sulfonate, sulfopropyl, phosphocellulose, P11 phosphocellulose, heparin sulfate, or a derivative or analog thereof;
(vi) the agent that interferes with the conversion of B-DNA to Z-DNA in the biofilm or its local environment; or
(vii) the method is performed in the absence of administration of a DNAse enzyme.

11. The method of claim 2, wherein one or more of the following applies:
(i) the agent is an inhibitor of polyamine synthesis or an agent that inhibits the binding of the polyamine to the DNA, wherein the agent is not an HMGB1 protein, fragment or an equivalent of each thereof;
(ii) the polyamine is selected from the group of: putrescine, spermine, cadaverine, 1,3-diaminopropane or spermidine;
(iii) the agent comprises a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof;
(iv) the agent comprises an agent that depletes cations from the biofilm, optionally a cation exchange resin, an aminopolycarboxylic acid, a crown ether, an azacrown, or a cryptand and optionally wherein the agent that depletes cations from the biofilm are from the group of: sulfonate, sulfopropyl, phosphocellulose, P11 phosphocellulose, heparin sulfate, or a derivative or analog thereof;
(v) the agent that interferes with the conversion of B-DNA to Z-DNA in the biofilm or its local environment; or
(vi) the method is performed in the absence of administration of a DNAse enzyme.

12. The method of claim 3 wherein one or more of the following applies:
(i) the agent is an inhibitor of polyamine synthesis or an agent that inhibits the binding of the polyamine to the DNA, wherein the agent is not an HMGB1 protein, fragment or an equivalent of each thereof;
(ii) the polyamine is selected from the group of: putrescine, spermine, cadaverine, 1,3-diaminopropane or spermidine;
(iii) the agent comprises a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof;
(iv) the agent comprises an agent that depletes cations from the biofilm, optionally a cation exchange resin, an aminopolycarboxylic acid, a crown ether, an azacrown, or a cryptand and optionally wherein the agent that depletes cations from the biofilm are from the group of: sulfonate, sulfopropyl, phosphocellulose, P11 phosphocellulose, heparin sulfate, or a derivative or analog thereof;
(v) the agent that interferes with the conversion of B-DNA to Z-DNA in the biofilm or its local environment; or
(vi) the method is performed in the absence of administration of a DNAse enzyme.

13. (canceled)

14. The method of claim 4, wherein one or more of the following applies:
(i) the agent is an inhibitor of polyamine synthesis or an agent that inhibits the binding of the polyamine to the DNA, wherein the agent is not an HMGB1 protein, fragment or an equivalent of each thereof;
(ii) the polyamine is selected from the group of: putrescine, spermine, cadaverine, 1,3-diaminopropane or spermidine;
(iii) the agent comprises a polyamine analog difluoromethylornithine, trans-4-methylcyclohexylamine, sardomozide, methylglyoxal-bis[guanylhydrazone] (MGBG), 1-aminooxy-3-aminopropane, oxaliplatin, cisplatin, dicyclohexylamine, a derivative of any thereof, or a salt thereof;
(iv) the agent comprises an agent that depletes cations from the biofilm, optionally a cation exchange resin, an aminopolycarboxylic acid, a crown ether, an azacrown, or a cryptand and optionally wherein the agent that depletes cations from the biofilm are from the group of: sulfonate, sulfopropyl, phosphocellulose, P11 phosphocellulose, heparin sulfate, or a derivative or analog thereof;
(v) the agent that interferes with the conversion of B-DNA to Z-DNA in the biofilm or its local environment; or
(vi) the method is performed in the absence of administration of a DNAse enzyme.

15-16. (canceled)

17. The method of claim 1, wherein the contacting is ex vivo and comprises coating a surface with an effective amount of one or more agents that deplete cations, wherein the agent is not an HMGB1 protein, fragment or an equivalent of each thereof.

18. (canceled)

19. The method of claim 17, wherein one or more of the following applies:
(a) the agent comprises an anti-B-DNA antibody or a fragment or derivative thereof;
(b) the agent comprises riboflavin, ethidium bromide, bis(methidium)spermine, daunorubicin, TMPyP4, a quaternary benzo[c]phenanthridine alkaloid, quinacrine, 9-amino acridine, or a derivative thereof; or
(c) the agent comprises chloroquine or a derivative thereof.

20-23. (canceled)

24. A method for treating a biofilm in a patient suffering from systemic lupus erythematosus (SLE) or cystic fibrosis (CF), comprising administering an effective amount of one or more of the following:
(a) an agent that interferes with the conversion of B-DNA to Z-DNA in the biofilm or its local environment, wherein the agent is not an HMGB1 protein, fragment or an equivalent of each thereof;
(b) one or more agents that interfere with the conversion of B-DNA to Z-DNA in the biofilm or its local environment, wherein the agent is not an HMGB1 protein, a fragment thereof or an equivalent of each thereof;
(c) HMGB1 protein or a biologically active fragment thereof and anti-B-DNA antibody or a fragment or derivative thereof; or
(d) chloroquine and anti-B-DNA antibody or fragment or derivative thereof.

25. (canceled)

26. The method of claim 24, wherein the agent comprises one or more of the following: chloroquine or a derivative thereof; an anti-B-DNA antibody or fragment or derivative thereof; riboflavin, ethidium bromide, bis(methidium)spermine, daunorubicin, TMPyP4, a quaternary benzo[c]phenanthridine alkaloid, quinacrine, 9-amino acridine, or a derivative thereof.

27-30. (canceled)

31. A method for treating a biofilm producing infection incident to administration of a platinum-based chemotherapy in a patient receiving or having received the chemotherapy, the method comprising administering an effective amount of one or more of the following:
(a) an agent that interferes with the conversion of B-DNA to Z-DNA in the biofilm or its local environment, wherein the agent is not an HMGB1 protein, fragment or an equivalent of each thereof;
(b) one or more agents that interfere with the conversion of B-DNA to Z-DNA in the biofilm or its local environment, wherein the agent is not an HMGB1 protein, a fragment thereof or an equivalent of each thereof;
(c) HMGB1 protein or a biologically active fragment thereof and anti-B-DNA antibody or a fragment or derivative thereof; or
(d) chloroquine and anti-B-DNA antibody or a fragment or derivative thereof.

32-34. (canceled)

35. The method of claim 31, wherein the agent comprises one or more of the following: chloroquine or a derivative thereof; an anti-B-DNA antibody or a fragment or derivative thereof; riboflavin, ethidium bromide, bis(methidium)spermine, daunorubicin, TMPyP4, a quaternary benzo[c]phenanthridine alkaloid, quinacrine, 9-amino acridine, or a derivative thereof.

36-37. (canceled)

38. The method of claim 1, further comprising contacting the biofilm with an effective amount of either or both of an agent that interferes with the binding of the eDNA to a DNA binding protein or an antibacterial agent.

39. The method of claim 38, wherein the agent that interferes with the binding of the eDNA to the DNA binding protein comprises one or more of an anti-DNABII antibody, an anti-IHF antibody or an anti-HU antibody, or fragments of each thereof.

40-41. (canceled)

42. The method of claim 14, wherein the agent that depletes cations from the biofilm has a net negative charge or a net neutral charge.

43. (canceled)

44. The method of claim 38, wherein the agent that interferes with the binding of the eDNA to a DNA binding protein has a net negative or net neutral or net positive charge.

45-47. (canceled)

48. A composition comprising one, two or three or more of: an agent that interferes with the binding of a polyamine to DNA in the biofilm, an agent that depletes cations from the biofilm, an agent that interferes with the conversion of B-DNA to Z-DNA in the biofilm or its local environment, an agent that interferes with the binding of the eDNA to a DNA binding protein or an antibacterial agent.

49-53. (canceled)

54. A kit comprising the composition of claim 48 and instructions for use, and optionally wherein the agents are combined or separately packaged.

55. (canceled)
